(19) United States
(12) Patent Application Publication
(10) Pub. No.: US 2024/0011099 A1
(43) Pub. Date: Jan. 11, 2024

(54) STAINED BIOLOGICAL SPECIMENS INCLUDING ONE OR MORE BIOMARKERS LABELED WITH ONE OR MORE DETECTABLE MOIETIES

(71) Applicant: Ventana Medical Systems, Inc., Tucson, AZ (US)

(72) Inventors: Daniel R. Bauer, Tucson, AZ (US); Mark Lefever, Quepos (CR); Larry Morrison, Oro Valley, AZ (US)

(21) Appl. No.: 18/224,446

(22) Filed: Jul. 20, 2023

Related U.S. Application Data
(63) Continuation of application No. PCT/EP2021/073738, filed on Aug. 27, 2021.
(60) Provisional application No. 63/141,091, filed on Jan. 25, 2021.

(51) Int. Cl. C12Q 1/6886; C12Q 1/6841; G01N 33/574; G01N 1/30

(52) U.S. Cl. CPC C12Q 1/6886; C12Q 1/6841; G01N 33/57492; G01N 1/30; G01N 2001/302

(57) ABSTRACT

The present disclosure is directed to a method of staining a biological specimen (e.g. a single serial tissue section derived from a biological sample) with one or more routine and/or special statins while concomitantly labeling the same biological specimen with one or more detectable moieties without the need for stripping any stain or evaluating different images of stained serial tissue sections of a biological specimen. In some embodiments, the present disclosure is directed to a biological specimen stained with one or more conventional dyes, and where the biological specimen further includes one or more biomarkers labeled with one or more detectable moieties.

### CROSS REFERENCE TO RELATED APPLICATION

The present application is a continuation of International Application No. PCT/EP2021/073738 filed on Aug. 27, 2021, which application claims the benefit of the filing date of U.S. Provisional Patent Application No. 63/141,091 filed on Jan. 25, 2021, the disclosure of which is hereby incorporated by reference herein in its entirety.

### FIELD OF THE DISCLOSURE

The present application is directed to a method of staining a biological specimen with one or more conventional dyes and labeling one or more biomarkers within the biological specimen with one or more detectable moieties, where the staining with the conventional dyes and the labeling of the biomarkers with the one or more detectable moieties occurs on the same biological specimen.

### BACKGROUND OF THE DISCLOSURE

Immunohistochemistry (IHC) refers to the processes of detecting, localizing, and/or quantifying antigens, such as a protein, in a biological sample using antibodies specific to the particular antigens. IHC provides the substantial advantage of identifying exactly where a particular protein is located within the biological sample. It is also an effective way to examine the tissues themselves. In situ hybridization (ISH) refers to the process of detecting, localizing, and quantifying nucleic acids. Both IHC and ISH can be performed on various biological samples, such as tissue (e.g. fresh frozen, formalin fixed, paraffin embedded) and cytological samples. Recognition of the targets can be detected using various labels (e.g., chromogenic, fluorescent, luminescent, radiometric), irrespective of whether the target is a nucleic acid or an antigen. To robustly detect, locate, and quantify targets in a clinical setting, amplification of the recognition event is desirable as the ability to confidently detect cellular markers of low abundance becomes increasingly important for diagnostic purposes. For example, depositing at the marker's site hundreds or thousands of label molecules in response to a single antigen detection event enhances, through amplification, the ability to detect that recognition event.

Adverse events often accompany amplification, such as non-specific signals that are apparent as an increased background signal. An increased background signal interferes with the clinical analysis by obscuring faint signals that may be associated with low, but clinically significant, expressions. Accordingly, while amplification of recognition events is desirable, amplification methods that limit increases in background signal are highly desirable. One such method is Tyramide Signal Amplification (TSA), which has also been referred to as catalyzed reporter deposition (CARD). U.S. Pat. No. 5,583,001 discloses a method for detecting and/or quantitating an analyte using an analyte-dependent enzyme activation system that relies on catalyzed reporter deposition to amplify the detectable label signal. Methods utilizing TSA effectively increase the signals obtained from IHC and ISH assays while not producing significant background signal amplification (see, for example, U.S. application publication No. 2012/0171668 which is hereby incorporated by reference in its entirety for disclosure related to tyramide amplification reagents). Reagents for these amplification approaches are being applied to clinically important targets to provide robust diagnostic capabilities.

TSA takes advantage of the reaction between horseradish peroxidase (HRP) and tyramide. In the presence of H₂O₂, tyramide is converted to a highly-reactive and short-lived radical intermediate that reacts preferentially with electron-rich amino acid residues on proteins. The short lifetime of the radical intermediate results in covalent binding of the tyramide to proteins on tissue in close proximity to the site of generation, giving discrete and specific signal. Covalently-bound detectable labels can then be detected by variety of chromogenic visualization techniques and/or by fluorescence microscopy.

Co-pending application PCT/EP2015/053556 entitled “Quinone Methide Analog Signal Amplification,” having an international filing date of Feb. 20, 2015, describes an alternative technique (“QMSA”) that, like TSA, may be used to increase signal amplification without increasing background signals. Indeed, PCT/EP2015/053556 describes novel quinone methide analog precursors and methods of using the quinone methide analog precursors in detecting one or more targets in a biological sample. There, the method of detection is described as comprising the steps of contacting the sample with a detection probe, then contacting the sample with a labeling conjugate that comprises an enzyme. The enzyme interacts with a quinone methide analog precursor comprising a detectable label, forming a reactive quinone methide analog, which binds to the biological sample proximally to or directly on the target. The detectable label is then detected.

### BRIEF SUMMARY OF THE DISCLOSURE

Different serial tissue sections derived from a single biological sample are traditionally used when staining a biological specimen with one or more conventional dyes (e.g. hematoxylin and eosin) and for the presence of one or more biomarkers. For instance, a first serial tissue section may be stained with hematoxylin and eosin to identify morphological features of the biological specimen; while one or more biomarkers in a second serial tissue section may be labeled with one or more chromogens or fluorophores in an IHC or ISH assay. As a result of the use of different serial tissue sections, there is no exact cell-to-cell and feature-to-feature correlation between the differently stained serial tissue sections.

To avoid the use of different serial tissue sections, routine and/or special stains, each described herein, may be applied to a biological specimen, imaged, and then stripped from the biological specimen. Subsequently, the same biological specimen, stripped of the routine or special stain, may be stained for the presence of one or more biomarkers, and then re-imaged. While this is feasible, such a process adds considerable time and effort, including the need to re-align the same regions for evaluation within any two generated images.

Applicant has developed a method of staining a biological specimen (e.g. a single serial tissue section derived from a biological sample) with one or more routine and/or special statins while concomitantly labeling the same biological specimen with one or more detectable moieties without the need for stripping any stain or evaluating different images of stained serial tissue sections of a biological specimen. Applicant submits that the methods described herein provide for a more accurate evaluation of stained biological specimens than the use of separate slides bearing separate serial tissue sections since separate serial tissue sections do not include the same cells, parts of cells, or tissue morphology.

In view of the foregoing, in some embodiments the present disclosure is directed to a biological specimen (e.g. a single biological specimen disposed on a substrate) stained with one or more conventional dyes, and where the biological specimen further includes one or more biomarkers labeled with one or more detectable moieties. In some embodiments, the one or more detectable moieties have a peak absorbance wavelength outside the visible spectrum, as described herein. In some embodiments, the one or more detectable moieties include a core selected from the group consisting of a coumarin core, a heptamethine cyanine core, and a croconate core. Non-limiting examples of suitable detectable moieties are described herein. In some embodiments, the one or more detectable moieties have peak absorbance wavelengths within either the ultraviolet spectrum or the infrared spectrum. In some embodiments, the one or more detectable moieties have peak absorbance wavelengths of less than about 430 nm or greater than about 670 nm. In some embodiments, the one or more conventional dyes are visible dyes, i.e. have one or more peak absorbance wavelengths within the visible spectrum, e.g. having one or more peak absorbance wavelengths ranging from between about 400 nm to about 700 nm, such as one or more peak absorbance wavelengths outside the photopic response of the human eye. In some embodiments, the conventional dyes are hematoxylin and/or eosin. In other embodiments, the conventional dyes include those utilized with a special stain.

In some embodiments, the present disclosure is also directed to methods of staining a biological specimen disposed on a substrate with one or more “routine stains” (e.g. hematoxylin and/or eosin) detectable within the visible spectrum and further labeling one or more biomarkers within the biological specimen with one or more detectable moieties which each have a peak absorbance wavelength that is outside the visible spectrum. In some embodiments, the one or more detectable moieties include a core selected from the group consisting of a coumarin core, a heptamethine cyanine core, and a croconate core. In some embodiments, the one or more detectable moieties have peak absorbance wavelengths within either the ultraviolet spectrum (including the near ultraviolet spectrum) or the infrared spectrum (including the near infrared spectrum). In some embodiments, the one or more detectable moieties each have a peak absorbance wavelength less than about 430 nm or greater than about 670 nm.

In some embodiments, the present disclosure is also directed to methods of staining a biological specimen disposed on a substrate with one or more “special stains” (e.g. stains for iron, mucins, glycogen, amyloid, nucleic acids, etc.) that are detectable generally within the visible spectrum for light microscopy and further labeling one or more biomarkers within the biological specimen with one or more detectable moieties which each have a peak absorbance wavelength that is outside the portion or portions of the spectrum of the special stain. In some embodiments, the one or more detectable moieties include a core selected from the group consisting of a coumarin core, a heptamethine cyanine core, and a croconate core. In some embodiments, the one or more detectable moieties have peak absorbance wavelengths within either the ultraviolet spectrum (including the near ultraviolet spectrum) or the infrared spectrum (including the near infrared spectrum). In some embodiments, the one or more detectable moieties each have a peak absorbance wavelength less than about 430 nm or greater than about 670 nm.

A first aspect of the present disclosure is a method of visualizing one or more targets within a biological specimen disposed on a substrate, comprising: labeling a first biomarker with a first detectable moiety, wherein the first detectable moiety has an absorbance maximum (λₘₐₓ) of either less than about 430 nm or greater than about 670 nm; and staining the biological specimen disposed on the substrate with at least one conventional dye having one or more peak absorbance wavelengths between about 400 nm and about 700 nm, wherein the peak absorbance wavelength of the first detectable moiety and the one or more peak absorbance wavelengths of the one or more conventional dyes are separated by at least 20 nm. In some embodiments, the one or more detectable moieties include a core selected from the group consisting of a coumarin core, a heptamethine cyanine core, and a croconate core.

In some embodiments, the first detectable moiety has a FWHM of less than about 200 nm. In some embodiments, the first detectable moiety has a FWHM of less than about 190 nm. In some embodiments, the first detectable moiety has a FWHM of less than about 180 nm. In some embodiments, the first detectable moiety has a FWHM of less than about 170 nm. In some embodiments, the first detectable moiety has a FWHM of less than about 160 nm. In some embodiments, the first detectable moiety has a FWHM of less than about 150 nm. In some embodiments, the first detectable moiety has a FWHM of less than about 140 nm. In some embodiments, the first detectable moiety has a FWHM of less than about 130 nm. In some embodiments, the first detectable moiety has a FWHM of less than about 120 nm. In some embodiments, the first detectable moiety has a FWHM of less than about 110 nm. In some embodiments, the first detectable moiety has a FWHM of less than about 100 nm. In some embodiments, the first detectable moiety has a FWHM of less than about 90 nm. In some embodiments, the first detectable moiety has a FWHM of less than about 80 nm. In some embodiments, the first detectable moiety has a FWHM of less than about 70 nm. In some embodiments, the first detectable moiety has a FWHM of less than about 60 nm. In some embodiments, the first detectable moiety has a FWHM of less than about 50 nm. In some embodiments, the first detectable moiety has a FWHM of less than about 40 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm. In some embodiments, the peak absorbance wavelength of the first detectable moiety and the one or more peak absorbance wavelengths of the one or more conventional dyes are separated by at least 40 nm. In some embodiments, the peak absorbance wavelength of the first detectable moiety and the one or more peak absorbance wavelengths of the one or more conventional dyes are separated by at least 15 nm. In some embodiments, the peak absorbance wavelength of the first detectable moiety and the one or more peak absorbance wavelengths of the one or more conventional dyes are separated by at least 20 nm. In some embodiments, the peak absorbance wavelength of the first detectable moiety and the one or more peak absorbance wavelengths of the one or more conventional dyes are separated by at least 25 nm. In some embodiments, the peak absorbance wavelength of the first detectable moiety and the one or more peak absorbance wavelengths of the one or more conventional dyes are separated by at least 30 nm. In some embodiments, the peak absorbance wavelength of the first detectable moiety and the one or more peak absorbance wavelengths of the one or more conventional dyes are separated by at least 35 nm. In some embodiments, the peak absorbance wavelength of the first detectable moiety and the one or more peak absorbance wavelengths of the one or more conventional dyes are separated by at least 40 nm. In some embodiments, the peak absorbance wavelength of the first detectable moiety and the one or more peak absorbance wavelengths of the one or more conventional dyes are separated by at least 45 nm. In some embodiments, the peak absorbance wavelength of the first detectable moiety and the one or more peak absorbance wavelengths of the one or more conventional dyes are separated by at least 50 nm. In some embodiments, the peak absorbance wavelength of the first detectable moiety and the one or more peak absorbance wavelengths of the one or more conventional dyes are separated by at least 55 nm. In some embodiments, the peak absorbance wavelength of the first detectable moiety and the one or more peak absorbance wavelengths of the one or more conventional dyes are separated by at least 60 nm. In some embodiments, the peak absorbance wavelength of the first detectable moiety and the one or more peak absorbance wavelengths of the one or more conventional dyes are separated by at least 70 nm. In some embodiments, the peak absorbance wavelength of the first detectable moiety and the one or more peak absorbance wavelengths of the one or more conventional dyes are separated by at least 80 nm. In some embodiments, the one or more detectable moieties include a core selected from the group consisting of a coumarin core, a heptamethine cyanine core, and a croconate core.

In some embodiments, the one or more conventional dyes include hematoxylin. In some embodiments, the one or more conventional dyes include eosin. In some embodiments, the one or more conventional dyes includes hematoxylin and eosin. In some embodiments, the one or more detectable moieties include a core selected from the group consisting of a coumarin core, a heptamethine cyanine core, and a croconate core.

In some embodiments, the one or more conventional dyes are selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red O, Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

In some embodiments, the labeling of the first biomarker with the first detectable moiety includes: (a) contacting the biological specimen with an anti-biomarker primary antibody; (b) contacting the biological specimen with an anti-specifies secondary antibody specific to the anti-biomarker primary antibody, wherein the anti-species antibody is conjugated directly or indirectly to at least one enzyme; and (c) contacting the biological specimen with a detectable conjugate comprising (i) the first detectable moiety, and (ii) a tyramide moiety, a quinone methide moiety, or a derivative or analog of a tyramide moiety or quinone methide moiety.

In some embodiments, the labeling of the first biomarker with the first detectable moiety includes: (a) contacting the biological specimen with an anti-biomarker primary antibody; (b) contacting the biological specimen with an anti-specifies secondary antibody specific to the anti-biomarker antibody, wherein the anti-species antibody is conjugated directly or indirectly to at least one enzyme; (c) contacting the biological specimen with a first tissue reactive conjugate comprising: (i) a first member of a pair of reactive functional groups capable of participating in a click chemistry reaction, and (ii) a tyramide moiety, a quinone methide moiety, or a derivative or analog of a tyramide moiety or quinone methide moiety; and (d) contacting the biological specimen with a detectable conjugate comprising (i) the first detectable moiety, and (ii) a second member of the pair of reactive functional groups.

In some embodiments, the first biomarker is a protein biomarker. In some embodiments, the first biomarker is selected from PD-L1, PD-1, Ki-67, CD3, CD8, Ki67, CD5, CD20, Pancytokeratin, HER2, ER, PR, p16, p63, p40, TTF-1, Napsin A, synaptophysin, and MART-1/MelanA. In some embodiments, the first biomarker is a nucleic acid biomarker. In some embodiments, the one or more detectable moieties include a core selected from the group consisting of a coumarin core, a heptamethine cyanine core, and a croconate core.

In some embodiments, the method further includes labeling a second biomarker with a second detectable moiety. In some embodiments, the second detectable moiety has a FWHM of less than about 200 nm (e.g. less than about 150 nm, less than about 100 nm, less than about 70 nm, etc.) and an absorbance maximum (λₘₐₓ) of either less than about 430 nm or greater than about 670 nm, and wherein the first and second detectable moieties are different. In some embodiments, the second detectable moiety is within the ultraviolet spectrum or within the infrared spectrum. In some embodiments, the second detectable moiety is within the ultraviolet spectrum. In some embodiments, the one or more detectable moieties include a core selected from the group consisting of a coumarin core, a heptamethine cyanine core, and a croconate core.

In some embodiments, the first and second detectable moieties have absorbance maximums (λₘₐₓ) that are separated by at least 10 nm. In some embodiments, the first and second detectable moieties have absorbance maximums (λₘₐₓ) that are separated by at least 15 nm. In some embodiments, the first and second detectable moieties have absorbance maximums (λₘₐₓ) that are separated by at least 20 nm. In some embodiments, the first and second detectable moieties have absorbance maximums (λₘₐₓ) that are separated by at least 30 nm. In some embodiments, the first and second detectable moieties have absorbance maximums (λₘₐₓ) that are separated by at least 35 nm. In some embodiments, the first and second detectable moieties have absorbance maximums (λₘₐₓ) that are separated by at least 40 nm. In some embodiments, the first and second detectable moieties have absorbance maximums (λₘₐₓ) that are separated by at least 45 nm. In some embodiments, the first and second detectable moieties have absorbance maximums (λₘₐₓ) that are separated by at least 50 nm. In some embodiments, the first and second detectable moieties include a core selected from the group consisting of a coumarin core, a heptamethine cyanine core, and a croconate core.

A second aspect of the present disclosure is a method of visualizing one or more targets within a biological specimen disposed on a substrate, comprising: labeling a first biomarker marker with a first detectable moiety, wherein the first detectable moiety includes a core selected from the group consisting of a coumarin core, a heptamethine cyanine core, and a croconate core; and staining the biological specimen disposed on the substrate with at least one conventional dye having one or more peak absorbance wavelengths between about 400 nm and about 700 nm, wherein the peak absorbance wavelength of the first detectable moiety and the one or more peak absorbance wavelengths of the one or more conventional dyes are separated by at least 20 nm.

In some embodiments, the first detectable moiety is within the ultraviolet spectrum. In some embodiments, the first detectable moiety is within the infrared spectrum.

In some embodiments, the first detectable moiety has a peak absorbance wavelength of less than about 430 nm. In some embodiments, the first detectable moiety has a peak absorbance wavelength of less than about 400 nm. In some embodiments, the first detectable moiety has a peak absorbance wavelength of greater than about 670 nm. In some embodiments, the first detectable moiety has a peak absorbance wavelength of greater than about 700 nm. In some embodiments, the first detectable moiety has a peak absorbance wavelength of less than about 430 nm but greater than about 400 nm, and wherein the one or more peak absorbance wavelengths of the one or more conventional dyes is greater than about 430 nm. In some embodiments, the first detectable moiety has a peak absorbance wavelength of greater than about 670 nm but less than about 700 nm, and wherein the one or more peak absorbance wavelengths of the one or more conventional dyes is less than about 670 nm.

In some embodiments, the method further includes labeling a second biomarker with a second detectable moiety. In some embodiments, the second detectable moiety has a FWHM of less than about 200 nm (e.g. less than about 150 nm, less than about 100 nm, less than about 70 nm, etc.) and an absorbance maximum (λₘₐₓ) of either less than about 430 nm or greater than about 670 nm, and wherein the first and second detectable moieties are different. In some embodiments, the second detectable moiety is within the ultraviolet spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm. In some embodiments, the first and second detectable moieties have absorbance maximums (λₘₐₓ) that are separated by at least 30 nm.

In some embodiments, the first detectable moiety is selected from the group consisting of:

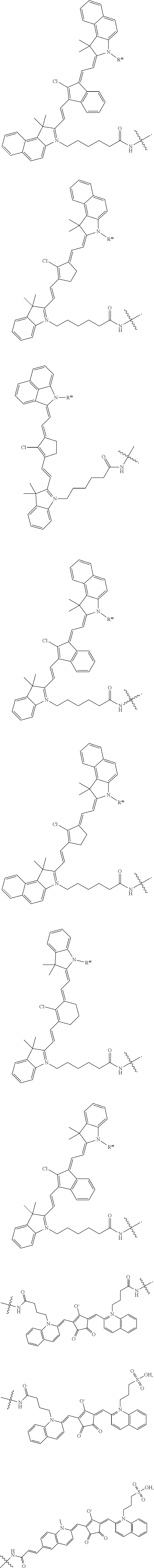

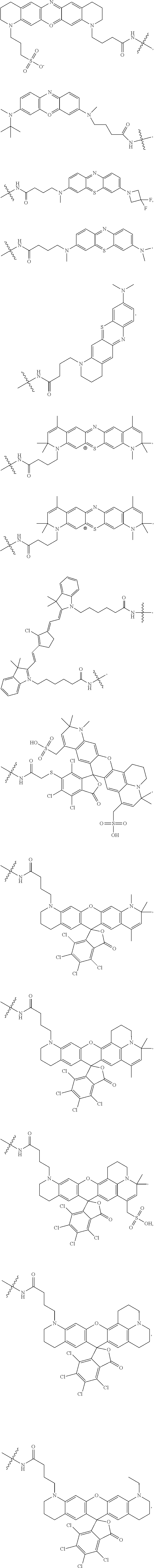

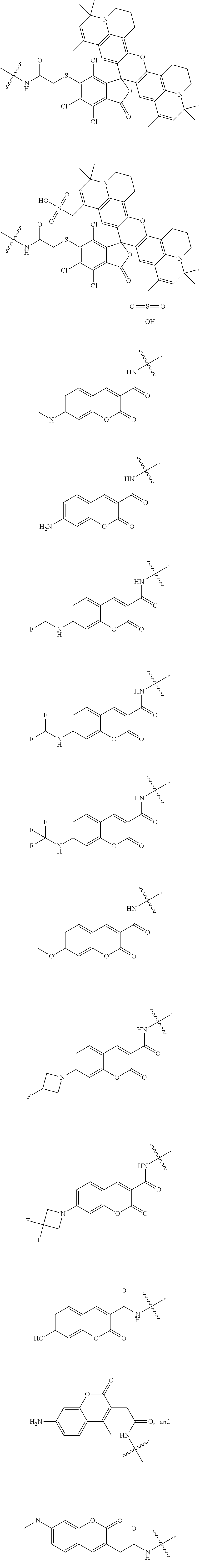

where the symbol “ [symbol] ” refers to the site in which the detectable moiety is conjugated to another moiety of a detectable conjugate.

A third aspect of the present disclosure is a stained biological specimen (e.g. a single stained serial tissue section) disposed on a substrate comprising a first biomarker labeled with a first detectable moiety; wherein the first detectable moiety has an absorbance maximum (λmax) of less than about 430 nm or greater than about 670 nm; and wherein the stained biological specimen is stained with at least one conventional dye, wherein the at least one conventional dye has one or more peak absorbance wavelengths within the visible spectrum.

In some embodiments, the first detectable moiety has a FWHM of less than about 200 nm. In some embodiments, the first detectable moiety has a FWHM of less than about 150 nm. In some embodiments, the first detectable moiety has a FWHM of less than about 100 nm. In some embodiments, the first detectable moiety has a FWHM of less than about 70 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm.

In some embodiments, the at least one conventional dye includes hematoxylin. In some embodiments, the at least one conventional dye includes eosin. In some embodiments, the at least one conventional dye includes hematoxylin and eosin. In some embodiments, the at least one conventional dye is selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

In some embodiments, the stained biological specimen further includes a second biomarker labeled with a second detectable moiety, wherein the second detectable moiety has a FWHM of less than about 200 nm (e.g. less than about 150 nm, less than about 100 nm, less than about 70 nm, etc.) and an absorbance maximum (λmax) of either less than about 430 nm or greater than about 670 nm, and wherein the first and second detectable moieties are different. In some embodiments, the second detectable moiety is within the ultraviolet spectrum or within the infrared spectrum. In some embodiments, the second detectable moiety is within the ultraviolet spectrum. In some embodiments, the first and second detectable moieties have absorbance maximums (λmax) that are separated by at least 20 nm. In some embodiments, the first and second detectable moieties have absorbance maximums (λmax) that are separated by at least 30 nm.

In some embodiments, the stained biological specimen further includes a third biomarker labeled with a third detectable moiety, wherein the third detectable moiety has a FWHM of less than about 200 nm (e.g. less than about 150 nm, less than about 100 nm, less than about etc.) and an absorbance maximum (λmax) of either less than about 430 nm or greater than about 670 nm, and wherein the first, second, and third detectable moieties are different. In some embodiments, the third detectable moiety is within the ultraviolet spectrum or within the infrared spectrum. In some embodiments, the third detectable moiety is within the ultraviolet spectrum. In some embodiments, the first, second, and third detectable moieties have absorbance maximums (λmax) that are separated by at least 20 nm. In some embodiments, the first, second, and third detectable moieties have absorbance maximums (λmax) that are separated by at least 30 nm.

A fourth aspect of the present disclosure is a stained biological specimen (e.g. a single serial tissue section) disposed on a substrate comprising a first biomarker labeled with a first detectable moiety; wherein the first detectable moiety has an absorbance maximum (λmax) of less than about 430 nm or greater than about 670 nm; wherein the stained biological specimen is stained with at least hematoxylin. In some embodiments, the stained biological specimen is further stained with eosin.

In some embodiments, the first detectable moiety has a FWHM of less than about 200 nm. In some embodiments, the first detectable moiety has a FWHM of less than about 150 nm. In some embodiments, the first detectable moiety has a FWHM of less than about 100 nm. In some embodiments, the first detectable moiety has a FWHM of less than about 70 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm.

In some embodiments, the stained biological specimen is further stained with at least one conventional dye other than hematoxylin and eosin. In some embodiments, the at least one conventional dye includes hematoxylin. In some embodiments, the at least one conventional dye is selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

In some embodiments, the stained biological specimen further includes a second biomarker labeled with a second detectable moiety, wherein the second detectable moiety has a FWHM of less than about 200 nm (e.g. less than about 150 nm, less than about 100 nm, less than about 70 nm, etc.) and an absorbance maximum (λmax) of either less than about 430 nm or greater than about 670 nm, and wherein the first and second detectable moieties are different. In some embodiments, the second detectable moiety is within the ultraviolet spectrum or within the infrared spectrum. In some embodiments, the second detectable moiety is within the ultraviolet spectrum. In some embodiments, the first and second detectable moieties have absorbance maximums (λmax) that are separated by at least 20 nm. In some embodiments, the first and second detectable moieties have absorbance maximums (λmax) that are separated by at least 30 nm.

A fifth aspect of the present disclosure is a stained biological specimen (e.g. a single serial tissue section) disposed on a substrate comprising a first biomarker labeled with a first detectable moiety; wherein the first detectable moiety has an absorbance maximum (λmax) of less than about 430 nm or greater than about 670 nm; wherein the stained biological specimen is stained with at least one special stain comprising one or more components detectable within the visible spectrum.

In some embodiments, the first detectable moiety has a FWHM of less than about 200 nm. In some embodiments, the first detectable moiety has a FWHM of less than about 150 nm. In some embodiments, the first detectable moiety has a FWHM of less than about 100 nm. In some embodiments, the first detectable moiety has a FWHM of less than about 70 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm.

In some embodiments, the special stain is a Van Gieson stain. In some embodiments, the special stain includes toludine blue. In some embodiments, the special stain includes alcain blue. In some embodiments, the special stain includes Masson's trichrome. In some embodiments, the special stain includes Azan trichrome. In some embodiments, the special stain includes acid fast.

In some embodiments, the first detectable moiety is selected from the group consisting of a coumarin core, a heptamethine cyanine core, and a croconate core.

In some embodiments, the stained biological specimen further includes a second biomarker labeled with a second detectable moiety, wherein the second detectable moiety has a FWHM of less than about 200 nm (e.g. less than about 150 nm, less than about 100 nm, less than about 70 nm, etc.) and an absorbance maximum (λmax) of either less than about 430 nm or greater than about 670 nm, and wherein the first and second detectable moieties are different. In some embodiments, the second detectable moiety is within the ultraviolet spectrum or within the infrared spectrum. In some embodiments, the second detectable moiety is within the ultraviolet spectrum. In some embodiments, the first detectable moiety is selected from the group consisting of a coumarin core, a heptamethine cyanine core, and a croconate core.

A sixth aspect of the present disclosure is a stained biological specimen disposed on a substrate comprising a first biomarker labeled with a first detectable moiety; wherein the first detectable moiety has an absorbance maximum (λmax) of less than about 430 nm or greater than about 670 nm; wherein the stained biological specimen is stained with at least one conventional dye, wherein the at least one conventional dye has one or more peak absorbance wavelengths within the visible spectrum, wherein the biological specimen is prepared by: contacting the biological specimen with a first primary antibody specific to the first biomarker; contacting the biological specimen with a first secondary antibody specific to the first primary antibody, wherein the first secondary antibody is conjugated to an enzyme; and contacting the biological specimen with a first detectable conjugate comprising (a) a tyramide moiety, a quinone methide moiety, or a derivative or analog of a tyramide moiety or quinone methide moiety; and (b) the first detectable moiety.

In some embodiments, the first detectable moiety has a FWHM of less than about 200 nm. In some embodiments, the first detectable moiety has a FWHM of less than about 150 nm. In some embodiments, the first detectable moiety has a FWHM of less than about 100 nm. In some embodiments, the first detectable moiety has a FWHM of less than about 70 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm.

In some embodiments, the one or more conventional dyes include hematoxylin. In some embodiments, the one or more conventional dyes include eosin. In some embodiments, the one or more conventional dyes includes hematoxylin and eosin. In some embodiments, the one or more conventional dyes are selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

In some embodiments, the first detectable moiety is within the ultraviolet spectrum. In some embodiments, the first detectable moiety is within the infrared spectrum. In some embodiments, the first detectable moiety has a peak absorbance wavelength of less than about 430 nm. In some embodiments, the first detectable moiety has a peak absorbance wavelength of less than about 400 nm. In some embodiments, the first detectable moiety has a peak absorbance wavelength of greater than about 670 nm. In some embodiments, the first detectable moiety has a peak absorbance wavelength of greater than about 700 nm. In some embodiments, the first detectable moiety has a peak absorbance wavelength of less than about 430 nm but greater than about 400 nm, and wherein the one or more peak absorbance wavelengths of the one or more conventional dyes is greater than about 430 nm.

In some embodiments, the stained biological specimen further includes a second biomarker labeled with a second detectable moiety; wherein the second detectable moiety has a FWHM of less than about 200 nm (e.g. less than about 150 nm, less than about 100 nm, less than about 70 nm, etc.) and an absorbance maximum (λmax) of less than about 430 nm or greater than about 670 nm. In some embodiments, the stained biological specimen is prepared by contacting the biological specimen with a second primary antibody specific to the second biomarker; contacting the biological specimen with a second secondary antibody specific to the second primary antibody, wherein the second secondary antibody is conjugated to an enzyme; and contacting the biological specimen with a second detectable conjugate comprising (a) a tyramide moiety, a quinone methide moiety, or a derivative or analog of a tyramide moiety or quinone methide moiety; and (b) the second detectable moiety.

A seventh aspect of the present disclosure is a stained biological specimen disposed on a substrate comprising a first biomarker labeled with a first detectable moiety; wherein the first detectable moiety has an absorbance maximum (λmax) of less than about 430 nm or greater than about 670 nm; wherein the stained biological specimen is stained with at least one conventional dye, wherein the at least one conventional dye has one or more peak absorbance wavelengths within the visible spectrum, wherein the biological specimen is prepared by: contacting the biological specimen with a first primary antibody specific to the first biomarker; contacting the biological specimen with a first secondary antibody specific to the first primary antibody, wherein the first secondary antibody is conjugated to an enzyme; contacting the biological specimen with a first tissue reactive moiety comprising (a) a tyramide moiety, a quinone methide moiety, or a derivative or analog of a tyramide moiety or quinone methide moiety; and (b) a first reactive functional group capable of participating in a click chemistry reaction; contacting the biological specimen with a first detectable conjugate comprising: (a) the first detectable moiety; and (b) a second reactive functional group.

In some embodiments, the first detectable moiety has a FWHM of less than about 200 nm. In some embodiments, the first detectable moiety has a FWHM of less than about 150 nm. In some embodiments, the first detectable moiety has a FWHM of less than about 100 nm. In some embodiments, the first detectable moiety has a FWHM of less than about 70 nm. In some embodiments, the FWHM of the first detectable moiety is less than about 60 nm. In some embodiments, the FWHM of the first detectable moiety is less than about 50 nm. In some embodiments, the FWHM of the first detectable moiety is less than about 40 nm.

In some embodiments, the one or more conventional dyes include hematoxylin. In some embodiments, the one or more conventional dyes include eosin. In some embodiments, the one or more conventional dyes includes hematoxylin and eosin. In some embodiments, the one or more conventional dyes are selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

In some embodiments, the first detectable moiety is within the ultraviolet spectrum. In some embodiments, the first detectable moiety is within the infrared spectrum. In some embodiments, the first detectable moiety has a peak absorbance wavelength of less than about 430 nm. In some embodiments, the first detectable moiety has a peak absorbance wavelength of less than about 400 nm. In some embodiments, the first detectable moiety has a peak absorbance wavelength of greater than about 670 nm. In some embodiments, the first detectable moiety has a peak absorbance wavelength of greater than about 700 nm. In some embodiments, the first detectable moiety has a peak absorbance wavelength of less than about 430 nm but greater than about 400 nm, and wherein the one or more peak absorbance wavelengths of the one or more conventional dyes is greater than about 430 nm.

In some embodiments, the stained biological specimen further includes a second biomarker labeled with a second detectable moiety; wherein the second detectable moiety has a FWHM of less than about 200 nm (e.g. less than about 150 nm, less than about 100 nm, less than about 70 nm, etc.) and an absorbance maximum (λmax) of less than about 430 nm or greater than about 670 nm. In some embodiments, the stained biological specimen is prepared by contacting the biological specimen with a second primary antibody specific to the first biomarker; contacting the biological specimen with a second secondary antibody specific to the second primary antibody, wherein the second secondary antibody is conjugated to an enzyme; contacting the biological specimen with a second tissue reactive moiety comprising (a) a tyramide moiety, a quinone methide moiety, or a derivative or analog of a tyramide moiety or quinone methide moiety; and (b) a first reactive functional group capable of participating in a click chemistry reaction; contacting the biological specimen with a second detectable conjugate comprising: (a) the second detectable moiety; and (b) a second reactive functional group.

### BRIEF DESCRIPTION OF THE FIGURES

The patent or application file contains at least one drawing executed in color. Copies of this patent or patent application publication with color drawings will be provided to the Office upon request and the payment of the necessary fee.

FIGS. 1A and 1B illustrate methods of detecting signals corresponding to one or more conventional dyes and one or more biomarkers labeled with one or more detectable moieties in a biological sample in accordance with one embodiment of the present disclosure.

FIG. 2A illustrates methods of labeling one or more biomarkers in a biological specimen with a detectable moiety in accordance with one embodiment of the present disclosure.

FIG. 2B illustrates methods of labeling one or more biomarkers in a biological specimen with a detectable moiety in accordance with one embodiment of the present disclosure.

FIG. 3 illustrates a method of detecting signals corresponding to one or more conventional stains and one or more biomarkers labeled with one or more detectable moieties in a biological sample, where the method utilizes detectable conjugates including (i) a detectable moiety, and (ii) a tyramide moiety, a derivative of a tyramide moiety, a quinone methide moiety, or a derivative of a quinone methide moiety, in accordance with one embodiment of the present disclosure.

FIG. 4 illustrates the deposition of a conjugate including a quinone methide moiety in accordance with one embodiment of the present disclosure.

FIG. 5 illustrates the deposition of a conjugate including a tyramide moiety in accordance with one embodiment of the present disclosure.

FIG. 6 illustrates a method of detecting signals corresponding to one or more conventional stains and one or more biomarkers in a biological sample, where the method utilizes detectable conjugates including (i) a detectable moiety, and (ii) reactive functional groups capable of participating in a click chemistry reaction, in accordance with one embodiment of the present disclosure.

FIG. 7 illustrates the deposition of a conjugate including a quinone methide moiety in accordance with one embodiment of the present disclosure.

FIG. 8 illustrates the deposition of a conjugate including a tyramide moiety in accordance with one embodiment of the present disclosure.

FIG. 9 illustrates the absorbance of hematoxylin and eosin and the corresponding visual response, showing that hematoxylin and eosin absorb across visible spectrum but absorbance is reduced in the deep blue/UV and minimal in the near IR.

FIGS. 10A-10C illustrate the peak absorbance wavelengths of several detectable moieties. In particular, FIGS. 10A and 10B illustrate the peak absorbance wavelengths of several detectable moieties as compared with hematoxylin and eosin.

FIG. 11 depicts monochrome images of light transmitted through a formalin-fixed paraffin-embedded (FFPE) normal pancreas tissue stained with hematoxylin plus eosin (“H&E”) and also stained in an IHC assay for the presence synaptophysin using Cy7 (cyanine 7) chromogen. The image on the left is illuminated with a 513 nm LED where absorbance is primarily due to eosin. The center image is illuminated with a 620 nm LED where absorbance is primarily due to hematoxylin. The image on the right is illuminated with a 770 nm LED where absorbance is primarily due to Cy7 and indicative of synaptophysin expression.

FIG. 12 provides color composite images of FFPE normal pancreas tissue stained with both synaptophysin IHC, using Cy7 chromogen, and H&E. The composite image on the left is formed from light transmitted at 513 nm (primarily absorbed by eosin) and 620 nm (primarily absorbed by hematoxylin) mimicking the visual appearance when viewed under white light through the microscope oculars. The composite image on the right is formed by adding the invisible light transmitted at 770 nm (absorbed by Cy7) and indicative of synaptophysin expression, to the H&E composite image on the left.

FIG. 13 illustrates monochrome images of light transmitted through an FFPE tonsil tissue stained with CD20 IHC, using DCC chromogen, CD8 IHC using Cy7 chromogen, and H&E. The image on the top left is illuminated with a 513 nm LED where absorbance is primarily due to eosin. The image on the top right is illuminated with a 620 nm LED where absorbance is primarily due to hematoxylin. The image on the lower left is illuminated with a 415 nm LED where absorbance is primarily due to DCC and indicative of CD20 expression. The image on the lower right is illuminated with a 770 nm LED where absorbance is primarily due to Cy7 and indicative of CD8 expression.

FIG. 14 sets forth color composite images of FFPE tonsil tissue stained with CD20 IHC, using DCC chromogen, CD8 IHC using Cy7 chromogen, and H&E. The composite image on the left is formed from light transmitted at 513 nm (primarily absorbed by eosin) and 620 nm (primarily absorbed by hematoxylin) mimicking the visual appearance when viewed under white light through the microscope oculars. The composite image in the middle is formed by adding the invisible light transmitted at 415 nm (absorbed primarily by DCC) and indicative of CD20 expression, pseudo-colored black, to the H&E composite image on the left. The composite image on the right is formed by adding the invisible light transmitted at 770 nm (absorbed by Cy7) and indicative of CD8 expression, pseudo-colored black, to the H&E composite image on the left.

FIG. 15 provides an absorbance spectrum of FFPE tonsil tissue stained with CD20 IHC using a DCC chromogen, CD8 IHC using the Cy7 chromogen, and H&E, showing that the DCC and Cy7 absorbance is well separated and distinguishable from the strong hematoxylin and eosin absorbance.

FIG. 16 illustrates monochrome images of light transmitted through an FFPE colon tumor tissue stained with CD3 IHC, using HCCA chromogen, CD8 IHC using Cy7 chromogen, and H&E. The image on the upper left is illuminated with a 390 nm LED where absorbance is primarily due to HCCA and indicative of CD3 expression. The image on the upper right is illuminated with a 770 nm LED where absorbance is due to Cy7 and indicative of CD8 expression. The image on the lower left is illuminated with a 513 nm LED where absorbance is primarily due to eosin. The image on the top right is illuminated with a 620 nm LED where absorbance is primarily due to hematoxylin.

FIG. 17 sets forth color composite images of FFPE colon tumor tissue stained with CD3 IHC, using HCCA chromogen, CD8 IHC using Cy7 chromogen, and H&E. The composite image on the upper left is formed from light transmitted at 513 nm (primarily absorbed by eosin) and 620 nm (primarily absorbed by hematoxylin) mimicking the visual appearance when viewed under white light through the microscope oculars. The composite image in the upper right is formed by adding the invisible light transmitted at 390 nm (absorbed primarily by HCCA) and indicative of CD3 expression, pseudo-colored black, to the H&E composite image. The H&E absorbance is lowered to improve contrast with the CD3 stain. The composite image on the lower left is formed by adding the invisible light transmitted at 770 nm (absorbed by Cy7) and indicative of CD8 expression, pseudo-colored black, to the H&E composite image. The H&E absorbance is reduced to improve contrast with the CD3 stain. The composite image in the lower right if formed by combing the CD3 monochrome image, pseudo-colored magenta, with the CD8 monochrome image, pseudo-colored cyan, thereby displaying CD3+/CD8− cells as magenta and CD3+/CD8+ cells as the additive color of blue due to CD3 and CD8 co-expression. Note the lack of cyan cells since CD8 is not expressed alone on t-cells.

FIG. 18 depicts an overlay of color camera (visible white light illumination) and monochrome camera (770 nm LED illumination) images using a 2-camera system with different color image opacities, recorded on a FFPE breast tumor xenograft stained with ERBB2 (HER2) IHC, using Cy7 chromogen, and H&E. The overlaid images from left to right progress from 100% color image opacity (only H&E stain visible) to 0% color image opacity (only Cy7 stain visible, identifying ERBB2 expression) with two intermediate levels of opacity.

FIG. 19 provides an absorbance spectrum of a cervical cytology specimen stained with conventional Papanicolaou (PAP) stain, showing that the PAP stain absorbs across visible spectrum, but absorbance is reduced in deep blue/UV and minimal in near IR.

FIG. 20 depicts images of a cervical cytology specimen stained with Ki67 IHC, using a DCC chromogen, p16 IHC, using the Cy7 chromogen, and PAP stain, recorded with the 2-camera system. The top left image was recorded with the color camera under white light illumination showing the PAP stain. The top center image was recorded with the monochrome camera illuminated with a 405 nm LED where absorbance is largely due to DCC and indicative of Ki67 expression. The top right image was recorded with the monochrome camera illuminated with a 770 nm LED where absorbance is due to Cy7 and indicative of p63 expression. The long cluster of cells in the lower right quadrant of the images shows expression of both Ki67 and p16 indicating abnormality. The lower images are color composites constructed by combining the Ki67 and p16 images with pseudo-coloring. The lower left image shows Ki67 pseudo-colored magenta and p16 pseudo colored cyan. Abnormal cells are darker and range in color from magenta to blue (the additive color) to cyan depending upon the relative expression levels in each cell. The lower right images shows Ki67 pseudo-colored red and p16 pseudo-colored to mimic the visual colors of the commercial assay using conventional chromogens.

FIG. 21 provides images of a cervical cytology slide stained with Ki67 IHC, using the DCC chromogen, p16 IHC, using the Cy7 chromogen, and PAP stain, recorded with the 2-camera system. The top left image was recorded with the color camera under white light illumination showing the PAP stain. The top center image was recorded with the monochrome camera illuminated with a 405 nm LED where absorbance is largely due to DCC and indicative of Ki67 expression. The top right image was recorded with the monochrome camera illuminated with a 770 nm LED where absorbance is due to Cy7 and indicative of p63 expression. The cluster of four cells in the center left portion of the image shows expression of both Ki67 and p16 indicating abnormality.

FIG. 22 illustrates the absorbance spectra of several conventional dyes, such as conventional dyes used in special stains, including Acid Fast Bacteria (AFB), Trichrome Blue (Tri Blue), Trichrome Green (Tri Green), Jones H&E Jones H E), and Jones Light Green (Jones L G)

FIG. 23 sets forth images of melanoma FFPE tissue stained both with IHC targeting MART-1/melan A, using the Cy7 chromogen, and H&E, recorded on the 2-camera system. The image on the left is a recorded with the color camera under white light illumination showing the H&E stain and the presence of brown melanin pigment. The image on the right is recorded with the monochrome camera illuminated with the 770 nm LED, showing MART1/melanA staining and reduced melanin interference.

FIG. 24 depicts images of FFPE tonsil tissue stained both with ISH targeting kappa mRNA, using the Cy7 chromogen, and H&E, recorded on the 2-camera system. The image on the left is a recorded with the color camera under white light illumination showing the H&E stain. The image on the right is recorded with the monochrome camera illuminated with the 770 nm LED, showing the presence of kappa mRNA.

FIG. 25 sets forth images of tonsil FFPE tissue stained with CD8 IHC, using Cy7-quinone methide chromogen in the top pair of images, and using AMCA-tyramide chromogen in the bottom pair of images. Using the 2-camera system, images recorded with the color camera under white light illumination are presented on the left sides, and images recorded with the monochrome camera using 770 nm illumination where Cy7 absorbs are presented on the right side, showing different chromogen chemistries can be utilized to deposit the invisible chromogens (note—other examples used click chemistry and tyramide—alkyne click partner).

FIG. 26A provides a flowchart illustrating multispectral imaging with a single monochrome camera. A number of illumination channels of different wavelengths are provided by (1) continuous light sources (e.g. tungsten, xenon, mercury, metal halide lamps) and optical filters selected to pass bands of light aligned with the absorbance of each chromogen or dye applied to the specimen, and/or (2) light emitting diodes (LEDs) with emission bands similarly aligned with chromogen and dye absorbance, with or without optical filters to further define the LED light emission. Additional light channels of wavelengths intermediate to neighboring chromogens may be used for over-sampling. The slide-mounted specimen is placed on the microscope stage and viewed with desired light channels or with white light from a continuous light source or a combination of LEDs mimicking white light, and a microscope field is selected for imaging (Step 1). In the process, light channel intensities and camera exposure times are selected to make use of the camera's dynamic range (Step 2). The specimen is then sequentially illuminated with the desired light channels while recording monochrome images of light transmitted through the specimen for each light channel (Step 3). If a region larger than a single microscope field is being recorded, e.g. a whole specimen or whole slide scan, then the stage is moved to adjacent field(s) within the desired region and the sequential illumination and imaging are repeated. The stage movements, illumination, and imaging may be coordinated by a computer to produce automatically recorded multi-spectral images of the single microscope field or the larger whole specimen or whole slide region (Step 4). For flat-fielding (correcting for illumination intensity differences across a microscopic field) or calculation of transmission and absorbance images, the imaging procedure is repeated for a blank region (no tissue) of the slide or an unstained tissue specimen at the same light intensities and exposure times (Step 5). The blank image may be recorded before or after imaging of the specimen, or may be performed before and after and the blank images averaged. Images are then processed (Step 6) as summarized in the ‘Image processing and composite image formation’ flowchart (FIG. 26E).

FIG. 26B provides a schematic of a multispectral imaging system utilizing a single monochrome camera.

FIG. 26C provides a flowchart setting forth dual camera viewing and imaging with color and monochrome cameras. To view and record images with the dual-camera system, position the specimen slide on the microscope stage (Step 1) and turn on the white light and the desired invisible illumination channels (Step 2). View live color images of the conventional stain and monochrome biomarker images simultaneously on the computer monitor (Step 3) and select image acquisition times for optimal exposure (Step 4). The two images can be overlaid (optional) and the opacity adjusted to identify the same cell(s) in both the monochrome and color images, observing biomarker expression within the full context of the conventional stain (Step 5). The full specimen can be evaluated with manual stage movements, as is commonly performed by a pathologist on a clinical specimen, viewing both the conventional stain and biomarker on the computer monitor, with the additional option of viewing the conventional stain directly through the oculars (Step 6). Color and monochrome images of individual specimen fields may be recorded for archiving purposes, composite image formation, and/or for quantitative analysis (Step 7). Image processing may be performed as illustrated in the ‘Image processing and composite image formation’ flowchart (FIG. 26E).

FIG. 26D provides a schematic for dual camera viewing and imaging with color and monochrome cameras. The dual camera system utilizes two cameras, one color and one monochrome, to simultaneously view and image conventional stains, absorbing light within the visible spectrum, and IHC-deposited chromogens, absorbing light outside or at the edge of the visible spectrum. It is believed that the key to the dual camera approach is the ability to combine broad band white light with invisible light bands at the illumination port of a microscope and then separating the visible and invisible light transmitted through the specimen, directing the visible light to the color camera and the invisible light to the monochrome camera. In the schematic illustrated in FIG. 26D, white light is generated by a tungsten continuous light source (A) using optical filters to remove the UV and far-red light and transmit only the visible light. A spectrum of tungsten lamp emission filtered to remove the near IR light is included (see FIG. 26D, plot A). Invisible light bands are generated with LEDs, with or without optical filtering to narrow the light bands, or a tungsten lamp combined with a filter wheel fitted with single bandpass filters (B). The visible white light and invisible light beams are combined with a partially reflective optical element (beam splitter, C). Spectra of a deep-blue/UV and a far-red/near IR emitting diode are included (see FIG. 26D, plot B) in the figure. An example of a partially reflective optical element is a glass plate with a neutral density coating (essentially wavelength independent) that reflects about 50% of light incident at about 45° and transmits about 50% of the light incident at about 45°. Alternatively, the reflective element may have a dichroic coating (wavelength dependent transmission/reflection) designed to transmit about 100% of visible light incident at about 45° and reflect about 100% of UV and far-red/near IR light incident at about 45°, for greater light throughput. The transmission spectrum of such a beam splitter is included (see FIG. 26D, plot C) in the figure. Relative reflection at different wavelengths can be controlled in the reflector design to change the relative amounts of each light band directed to the specimen. After passing through the specimen and objective, the light is split into two portions, each portion directed to a different camera, using another partially reflective optical element that may have the same spectral properties as the first beam splitter. In the case of the about 50% neutral density beam splitter, half of the light is transmitted to the color camera and half is reflected to the monochrome camera (essentially independent of wavelength). In the case of the dichroic beam splitter, the visible light is transmitted to the color camera and the UV and far-red/near IR light is reflected to the monochrome camera. Optical filters D and E are required with the neutral density beam splitters to ensure only visible light reaches the color camera and only UV and far-red/near IR light reaches the monochrome camera, respectively. Plots of potential optical filter transmission spectra are provided in the figure (see FIG. 26, plots D and E), corresponding to color camera (D) and monochrome camera (E) filters, respectively. Images of microscope fields acquired by each camera are presented on the computer monitor. Images can be acquired at video rates so the visible light, viewed in color as it looks when viewed through the oculars by the microscopist, and invisible light, viewed as a monochrome image, are presented side-by-side, or overlaid. Viewing by eye through the oculars is also available, with optical filtering at the eyepiece (F) to transmit only visible light from the tungsten microscope lamp and block the invisible light (see FIG. 26D, transmission plot F), providing additional eye protection. Alternatively, the oculars may be replaced with a tube lens that does not permit viewing by eye. Note that other continuous light sources could be employed, for example xenon or mercury arc lamps or metal halide lamps or combinations of visible LEDs. Other invisible light sources can be used including continuous light sources optically filtered with bandpass filters and laser diodes. Also, some applications use two monochrome cameras, each displaying a different biomarker expression pattern, or two color cameras with different optical filtering, instead of one of each camera type. As with the single monochrome camera system (see FIG. 26A), images of multiple single microscope fields can be combined into larger regions of interest, including whole-specimen and whole-slide scans. The dual-camera system can be used as a single monochrome camera system by only utilizing the monochrome camera with neutral density beam splitters, or with the beam splitters removed, or using 100% reflective mirrors depending upon the mounting position of the monochrome camera, in order to provide access to the full light spectrum.

FIG. 26E sets forth a flowchart illustrating the processing of images and their combining into composite images. The number of illumination channels (λ1, λ2, . . . λn) utilized (n) is minimally equal to the number of different stains (S1, S2, . . . Sn) used in the multiplex IHC, where the stains include chromogens and conventional staining components (e.g. hematoxylin and eosin). The illumination channels are selected to emphasize each stain, for example, to locate an illumination channel near the peak absorbance of each stain. Additional channels located between the absorbance peaks may be added to improve unmixing, referred to as over-sampling (not depicted in FIG. 26E). The first step is to generate transmission (T) images for each illumination channel by dividing the images of light transmitted through the specimen at each channel (Specimen image, λ1, etc.) by the corresponding blank images (no tissue or unstained tissue; Blank image, λ1, etc.). The pixel values of the specimen images represent the intensity of light transmitted through the specimen at that location (I), and the pixel values of the blank images represent the intensity of light incident on the specimen at that location (I0). All operations are performed on a pixel-by-pixel basis. The resulting pixel values of the T images should range between 0 and 1 (I/I0), requiring floating point pixel values. Logarithms are taken of the T image pixels and the pixels multiplied by −1 to provide the absorbance (A) images (A=−log[T]). Absorbance, also referred to here as optical density (OD), is valuable since it is proportional to concentration according to Beer's Law, and is necessary for quantification and the linear algorithms used in spectral unmixing. Spectral unmixing is a process by which “pure” individual images, containing absorbance only due to one stain are generated by removing the spectral contributions from other stains with overlapping absorbance spectra (overlapping absorbance is commonly referred to as spectral cross-talk). These images of pure stains after cross-talk correction are referred to here as unmixed images (U-image, S1, etc.). If the absorbance peaks of various stains used in a multiplex assay are well separated by wavelength, cross-talk may be too small to require unmixing in order to provide values representative of the single stain concentrations, however, as multiplexing order is increased and spectral separation is reduced, unmixing becomes a necessity. Being proportional to stain concentration, the U images may be used directly for quantification, similar to fluorescence images for which the pixel values are also proportional to fluorescent stain concentration. Spectral unmixing procedures are described in Morrison L E, Lefever M R, Behman L J, Leibold T, Roberts E A, Horchner U B, Bauer D R (2020) Brightfield Multiplex Immunohistochemistry with Multispectral Imaging. Lab Invest. https://doi.org/10.1038/s41374-020-0429-0 and references therein. The U images may additionally be used to form composite images. The A images prior to unmixing can be used for composite image formation if cross-talk is sufficiently small to provide acceptable composites, saving the additional computation time and added complexity required for spectral unmixing (see previous comment). The U images corresponding to each stain (S1, S2, . . . Sn) are copied twice to form the contributions of each stain to the R, G, and B planes of the final composite image, labeled R-image S1, R-image S2, . . . , G-image S1, G-image S2, . . . , and B-image S1, B-image S2, . . . , in the flow chart. Each image copy is multiplied by an amplification factor, ‘a,’ and a color-weighting factor, ‘C.’ Amplification factors less than one decrease a stain's contribution to the final composite image and amplification factors greater than one increase a stain's contribution to the composite image. Color weighting factors provide the stain's pseudocolor in a composite image and range from 0 to 1 for the red, green, and blue component of each stains color. For example, if stain 1 (S1) is to be pseudocolored red, then the color weighting factors are CR1=1, CG1=0, and CB1=0 in a fluorescence-like composite image representation for generating the R-, G-, and B-images, respectively. For brightfield-like image representations, red color is produced by weighting factors of CR1=0, CG1=1, and CB1=1, or 1 minus the fluorescence-like weighting factor for the same color. To create the final red image plane of the composite image (R-plane), the R-images for each stain are summed. The final green and blue image planes (G-plane and B-plane) are similarly created by summing the G- and B-images of each stain, respectively. For a fluorescence-like color representation (values proportional to concentration and absorbance, or A-composite image), the three color planes can be scaled by dividing each plane by the maximum value of the collective three color planes. The final images are multiplied by 255 for 8-bit color image planes. For a brightfield color representation (transmission or T-composite image), the summed R-, G-, and B-images are each multiplied by −1 and the antilogarithms taken. The final image planes are multiplied by 255 for 8-bit color image planes.

FIG. 27 provides absorbance spectra recorded on FFPE lung tissues stained with mucicarmine special stain showing that while the mucicarmine stain absorbance may interfere with the use of iIHC chromogens absorbing in the deep-blue/UV spectral region, the mucicarmine absorbance should not interfere with iIHC chromogens absorbing in the far-red/near-IR spectral region.

FIG. 28 depicts images recorded on the dual-camera system of lung tumor FFPE tissues stained simultaneously with TTF-1 (IR870 iCDC) plus p40 (Cy7 iCDC) dual iIHC and mucicarmine special stain. The visible absorbance of the mucicarmine special stain (left images) was recorded with the color camera and the far-red/near-IR images (middle and right images) were recorded with the monochrome camera. The top images were recorded on adenocarcinoma tissue and show expression of TTF-1 (right image) and mucin (pink staining of cytoplasm in TTF-1 expressing cells, left image) confirming the adenocarcinoma assignment. The lower images were recorded on squamous cell carcinoma tissue and show p40 positive cells and minimal mucin production, confirming the squamous cell carcinoma assignment.

FIG. 29 illustrates spectral unmixing to remove background signals from neighboring chromogenic and conventional stains. The absorbance spectra of various dyes used in a multiplex assays may have significant overlap (for example see spectra in FIG. 10) causing their absorbance to be visible in images intended to record only the absorbance of a single stain. Spectral unmixing is used to correct for this overlap and reduce or remove the interfering signals. This is seen by comparing the absorbance images of HCCA and Cy7 chromogens (left and right sides of figure, respectively) prior to spectral unmixing (top images) to the images after spectral unmixing (lower images). The unmixed HCCA image shows significant reduction in signal from hematoxylin-stained nuclei. The unmixed Cy7 image does not show considerable change since hematoxylin absorbs little in the spectral region where Cy7 absorbs.

FIG. 30 illustrates multispectral imaging and image processing: composite color images and spectral unmixing in NSCLC ADC FFPE tissue plus p40/TTF-1 duplex IHC. Panel A—Color composite image formed from spectrally unmixed monochrome transmitted light images recorded at 510 nm, where eosin primarily absorbs light, and 599 nm, where hematoxylin absorbs light (monochrome images not shown). Panels B and C—Monochrome camera images of transmitted light at 769 nm where Cy7 CDC primarily absorbs light, staining p40 (B), and 880 nm, where ir870 CDC absorbs light, staining TTF-1 (C). Panels E and F—spectrally unmixed images of p40 (Panel E) and TTF-1 (Panel F). Panel D—Two-color composite image formed from the spectrally unmixed hematoxylin (not shown) and TTF-1 (Panel F) images. Images were recorded using a 20× objective.

### DETAILED DESCRIPTION

Disclosed herein are detectable moieties and detectable conjugates comprising one or more detectable moieties. In some embodiments, the disclosed detectable moieties have a narrow wavelength and are suitable for multiplexing.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein, alkaline phosphatase (AP) is an enzyme that removes (by hydrolysis) and transfers phosphate group organic esters by breaking the phosphate-oxygen bond, and temporarily forming an intermediate enzyme-substrate bond. For example, AP hydrolyzes naphthol phosphate esters (a substrate) to phenolic compounds and phosphates. The phenols couple to colorless diazonium salts (chromogen) to produce insoluble, colored azo dyes.

As used herein, the term “antibody,” occasionally abbreviated “Ab,” refers to immunoglobulins or immunoglobulin-like molecules, including by way of example and without limitation, IgA, IgD, IgE, IgG and IgM, combinations thereof, and similar molecules produced during an immune response in any vertebrate, (e.g., in mammals such as humans, goats, rabbits and mice) and antibody fragments that specifically bind to a molecule of interest (or a group of highly similar molecules of interest) to the substantial exclusion of binding to other molecules. Antibody further refers to a polypeptide ligand comprising at least a light chain or heavy chain immunoglobulin variable region which specifically recognizes and binds an epitope of an antigen. Antibodies may be composed of a heavy and a light chain, each of which has a variable region, termed the variable heavy (VH) region and the variable light (VL) region. Together, the VH region and the VL region are responsible for binding the antigen recognized by the antibody. The term antibody also includes intact immunoglobulins and the variants and portions of them well known in the art.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids and proteins.

As used herein, the term a “biological specimen” can be any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological specimen can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease). A biological specimen can also be a sample obtained from any organ or tissue (including a biopsy or autopsy specimen, such as a tumor biopsy) or can include a cell (whether a primary cell or cultured cell) or medium conditioned by any cell, tissue or organ. In some examples, a biological specimen is a nuclear extract. In certain examples, a sample is a quality control sample, such as one of the disclosed cell pellet section samples. In other examples, a sample is a test sample. Samples can be prepared using any method known in the art by of one of ordinary skill. The samples can be obtained from a subject for routine screening or from a subject that is suspected of having a disorder, such as a genetic abnormality, infection, or a neoplasia. The described embodiments of the disclosed method can also be applied to samples that do not have genetic abnormalities, diseases, disorders, etc., referred to as “normal” samples. Samples can include multiple targets that can be specifically bound by one or more detection probes.

As used herein, the term “conjugate” refers to two or more molecules or moieties (including macromolecules or supra-molecular molecules) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules moieties.

As used herein, the terms “couple” or “coupling” refers to the joining, bonding (e.g. covalent bonding), or linking of one molecule or atom to another molecule or atom.

As used herein, the term “detectable moiety” refers to a molecule or material that can produce a detectable (such as visually, electronically or otherwise) signal that indicates the presence (i.e. qualitative analysis) and/or concentration (i.e. quantitative analysis) of the label in a sample.

As used herein, horseradish peroxidase (HRP) is an enzyme that can be conjugated to a labeled molecule. It produces a colored, fluorometric, or luminescent derivative of the labeled molecule when incubated with a proper substrate, allowing it to be detected and quantified. HRP acts in the presence of an electron donor to first form an enzyme substrate complex and then subsequently acts to oxidize an electronic donor. For example, HRP may act on 3,3′-diaminobenzidinetrahydrochloride (DAB) to produce a detectable color. HRP may also act upon a labeled tyramide conjugate, or tyramide like reactive conjugates (i.e. ferulate, coumaric, caffeic, cinnamate, dopamine, etc.), to deposit a colored or fluorescent or colorless reporter moiety for tyramide signal amplification (TSA).

As used herein, the term “immunohistochemistry” (IHC), refers to a method of determining the presence or distribution of an antigen in a sample by detecting interaction of the antigen with a specific binding agent or moiety, such as an antibody. A sample including an antigen (such as a target antigen) is incubated with an antibody under conditions permitting antibody-antigen binding. Antibody-antigen binding can be detected by means of a detectable label conjugated to the antibody (direct detection) or by means of a detectable label conjugated to a secondary antibody, which is raised against the primary antibody (e.g., indirect detection). Detectable labels include, but are not limited to, radioactive isotopes, fluorochromes (such as fluorescein derivatives, and rhodamine derivatives), enzymes and chromogenic molecules.

As used herein, the term “in situ hybridization” (ISH) type of hybridization that uses a labeled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH). This is distinct from immunohistochemistry, which localizes proteins in tissue sections. DNA ISH can be used to determine the structure of chromosomes, such as for use in medical diagnostics to assess chromosomal integrity. RNA ISH (hybridization histochemistry) is used to measure and localize mRNAs and other transcripts within tissue sections or whole mounts. For hybridization histochemistry, sample cells and tissues are usually treated to fix the target transcripts in place and to increase access of the probe to the target molecule. As noted above, the probe is either a labeled complementary DNA or a complementary RNA (Riboprobe). The probe hybridizes to the target sequence at elevated temperature, and then the excess probe is washed away (optionally hydrolyzed using RNase in the case of unhybridized, excess RNA probe). Solution parameters, such as temperature, salt and/or detergent concentration, can be manipulated to remove any non-identical interactions (i.e. only exact sequence matches will remain bound). Then, the labeled probe having been labeled effectively, such as with either radio-, fluorescent- or antigen-labeled bases (e.g., digoxigenin), is localized and potentially quantified in the tissue using either autoradiography, fluorescence microscopy or immunohistochemistry, respectively.

As used herein, the terms “multiplex,” “multiplexed,” or “multiplexing” refer to detecting multiple targets in a sample concurrently, substantially simultaneously, or sequentially. Multiplexing can include identifying and/or quantifying multiple distinct nucleic acids (e.g., DNA, RNA, mRNA, miRNA) and polypeptides (e.g., proteins) both individually and in any and all combinations.

As used herein, a “quinone methide” is a quinone analog where one of the carbonyl oxygens on the corresponding quinone is replaced by a methylene group (—CH₂—) to form an alkene.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant at least 10⁻³ M greater, 10⁻⁴ M greater or 10⁻⁵ M greater than a binding constant for either of the two members of the binding pair with other molecules in a biological specimen). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “target” refers to any molecule for which the presence, location and/or concentration is or can be determined. Examples of target molecules include proteins, nucleic acid sequences, and haptens, such as haptens covalently bonded to proteins. Target molecules are typically detected using one or more conjugates of a specific binding molecule and a detectable label.

As used herein, the symbol “ [symbol] ” refers to a location a moiety is bonded to another moiety.

Overview

Applicant has developed a method of staining a biological specimen with one or more conventional dyes (such as in “routine staining” or “special staining”) and labeling one or more biomarkers (in an IHC or ISH assay) within the biological specimen with one or more detectable moieties, where the staining with the conventional dyes and the labeling of the biomarkers with the one or more detectable moieties occurs on the same biological specimen (such as a single tissue section disposed on a substrate). Use of the detectable moieties described herein in conjunction with the conventional dyes facilitates the detection of the one or more labeled biomarkers without interfering with the interpretation of the one or more conventional dyes.

Hematoxylin plus eosin (H&E) is the most common histological stain and provides one of the most important cancer diagnostics. In simple terms, the hematoxylin in H&E binds regions of DNA content, imparting a blue color to all nuclei, while the eosin stains cytoplasm and connective tissue a pink color. In reality, H&E staining is considerably more nuanced, providing complex staining patterns and colorations that make it possible to distinguish numerous cellular and extracellular features. Pathologists gain an incredible amount of information from H&E stained tissues that supports diagnosis, prognosis, and prediction of therapeutic response.

For example, one study used 10 different histologic features of H&E stained non-small cell lung cancer (NSCLC) biopsies of patients undergoing immune-therapy to develop a new pathologic response criteria (see Cottrell T R, Thompson E D, Forde P M, Stein J E, Duffield A S, Anagnostou V, et al. Pathologic features of response to neoadjuvant anti-PD-1 in resected non-small-cell lung carcinoma: a proposal for quantitative immune-related pathologic response criteria (irPRC). Ann Oncol 2018; 29:1853-1860). In another study the same group evaluated 14 histologic features in H&E stained melanoma biopsy specimens from immune-therapy-treated patients and showed significant association with objective response and overall survival (see Stein J E, Soni A, Danilova L, Cottrell T R, Gajewski T F, Hodi F S, et al. Major pathologic response on biopsy (MPRbx) in patients with advanced melanoma treated with anti-PD-1: evidence for an early, on-therapy biomarker of response. Ann Oncol 2019; 30; 589-596). Pathologists are believed to be very comfortable with brightfield microscopy and evaluating H&E stained tissue, with considerable training being devoted to this during pathology residency. The importance of H&E staining is attested to by its practice for well over a century. Other conventional brightfield stains, such as Papanicoulou (PAP) staining of cervical specimens, and special stains such as giemsa, elastic, mucicarmine, and trichrome stains have long histories of valued use (see Chantziantoniou N, Donnelly A, Mukherjee M, Boon M E, Austin R M. Inception and Development of the Papanicolaou stain method. Acta Cytol 2017; 61:266-280; and Wick M R. Histochemistry as a tool in morphological analysis: a historical review. Ann Diagn Pathol 2012; 16:71-78).

Immunohistochemistry allows for staining of particulars molecular species, commonly proteins, through highly specific antibody reagents, and in combination with H&E, have greatly strengthened the pathologist's diagnostic capability (see Jaffer S, Bleiweiss I J. Beyond hematoxylin and eosin—the role of immunohistochemistry in surgical pathology. Cancer Invest 2004; 22:445-65). The antibodies direct chromogen deposition via enzyme-antibody conjugates and enzyme-catalyzed chromogen deposition reactions. Since IHC can stain specific proteins, as opposed to the relatively non-specific protein staining of eosin, under and over expression of important proteins, such as cell cycle and other cell signaling proteins, can be identified and used to classify tumor types, establish prognosis, and predict therapeutic response, adding considerably more information to that provided by H&E and other conventional stains.

For example, H&E can establish the presence of non-small cell lung cancer (NSCLC), but often expression of p16, TTF-1, cytokeratins 5 and 6, and/or Napsin A are required to clearly classify the cancer as squamous cell carcinoma (SSC) or adenocarcinoma (Ad Ca) (see Kerr K M, Bubendorf L, Edelman M J, Marchetti A, Mok T, Novello S, et al. Second ESMO consensus conference on lung cancer: pathology and molecular biomarkers for non-small-cell lung cancer. Ann Oncol 2014; 25:1681-1690; and Roberts E A, Morrison L E, Behman L J, Draganova-Tacheva R, O'Neill R5, Solomides C C. Chromogenic immunohistochemical quadruplex provides accurate diagnostic differentiation of non-small cell lung cancer. Ann Diagn Pathol 2019; 45:151454). In breast cancer, over-expression or gene amplification of the epithelial growth factor receptor II (HER2) is prognostic and predicts effectiveness of HER2 antagonists, such as trastuzumab, while over-expression of estrogen receptor (ER) and progesterone receptor (PR) proteins are prognostic and predict response to estrogen antagonists, such as tamoxifen (see Ross J, Fletcher J A. The HER-2/neu oncogene in breast cancer: prognostic factor, predictive factor, and target for therapy. Oncologist 1998; 3; 237-252; Vogel C L, Cobleigh M A, Tripathy D, Gutheil J C, Harris L N, Fehrenbacher L, et al. Efficacy and safety of trastuzumab as a single agent in first-line treatment of HER2-overexpressing metastatic breast cancer. J Clin Onc 2002; 20; 719-726; and Nasrazadani A, Thomas R A, Oesterreich S, Lee A V. Precision medicine in hormone receptor-positive breast cancer Frontiers Onc 2018; 8; article 144).

Unfortunately, complete analysis of a particular tumor by H&E and IHC may require more tissue than is available from a particular biopsy, especially for needle biopsies and fine needle aspirates, and from cytology specimens, such as cervical brushings and urine, with low cellularity. This is because one slide is required for H&E to make the cancer diagnosis, and one additional slide is required for each IHC stain, by common clinical practice. IHC multiplexing can increase the number of IHC staining reactions per slide, but still requires at least one slide in addition to the H&E stained slide. De-staining the H&E slide followed by IHC is possible, but complete removal of eosin and hematoxylin may prove difficult and considerably more time is required to perform the staining and evaluation sequentially for both the H&E and IHC. Additionally, the comparison of the same exact regions between the two staining methods requires imaging after H&E and again after IHC and locating and aligning the regions of interest.

Even when sufficient sample for several slide preparations is available, coordinating the evaluation of the H&E staining pattern and IHC protein expression patterns is problematic. For tissues, the best case is performing the H&E and IHC on adjacent (serial) tumor sections. However, tissue morphology changes with distance through the tumor specimen, and even serial sections show significant change in the shape and orientation of various tissue and cellular features. The same regions must be located on each slide, with changes in tumor with section cutting accounted for, and placement of cells with informative protein expression on one slide must be mentally aligned with H&E features on the other slide. Since cells on one section are not present or are only partially present (microtome cuts within cell) on the serial section, exact alignment of H&E and IHC information is rarely possible. The situation is worse for cytology specimens, for which alignment of information between two different specimen slides is not a possibility.

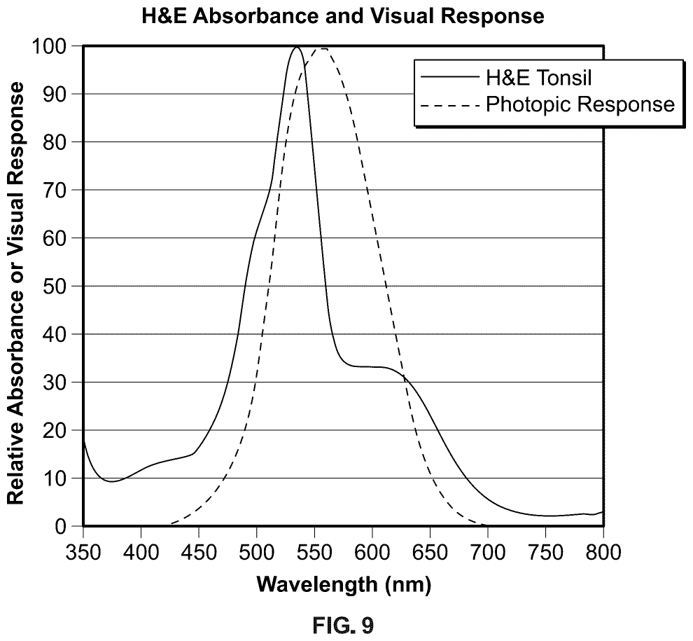

Multiplexing H&E staining with IHC on the same slide would be a solution to the above problems of limited specimen and alignment of information retrieved from different specimen slides. However, this has not been possible because H&E staining is so intense and covers the entire visible region of the light spectrum such that the chromogen stains will be obscured and difficult or impossible to read. FIG. 9 shows the absorbance spectrum of an H&E stained formalin fixed paraffin embedded (FFPE) tonsil tissue section. Also plotted is the human visual response, showing that the H&E stain absorbs across the visible range. A very dark black or brown chromogen, such as DAB would be more visible, but then the DAB obscures the H&E stain, so both H&E and IHC are poorly evaluated.

As noted above, Applicant has discovered that one or more conventional dyes introduced to a biological specimen may still be interpreted even when one or more of biomarkers within the biological specimen are labeled with one or more of the detectable moieties described herein (e.g. labeled with two or more detectable moieties, labeled with three or more detectable moieties, labeled with four or more detectable moieties, labeled with five or more detectable moieties, labeled with six or more detectable moieties, etc.).

Electromagnetic Spectrum

The electromagnetic spectrum is the range of frequencies (the spectrum) of electromagnetic radiation and their respective wavelengths and photon energies. This frequency range is divided into separate bands, and the electromagnetic waves within each frequency band are called by different names; beginning at the low frequency (long wavelength) end of the spectrum these are: radio waves, microwaves, infrared, visible light, ultraviolet, X-rays, and gamma rays at the high-frequency (short wavelength) end.

There are no precisely defined boundaries between the bands of the electromagnetic spectrum; rather the bands of the electromagnetic spectrum “fade” into each other. As a result, there exists overlap between the different bands of the electromagnetic spectrum. For instance, there exists overlap between the visible spectrum and ultraviolet spectrum and between the visible spectrum and the infrared spectrum. In particular, such overlap occurs in wavelength ranges adjacent to and/or encompassing the “visible spectrum,” such as in the wavelength ranges between about 370 nm and about 430 nm and the wavelength ranges between about 670 nm and about 730 nm.

In view of the foregoing, as used herein the term “visible spectrum” refers a spectrum of wavelengths that range from about 400 nm to about 700 nm. As used herein, the term “ultraviolet spectrum” includes wavelengths less than about 430 nm. As such, the “ultraviolet spectrum” includes near-ultraviolet wavelengths, i.e. those wavelengths adjacent to the “visible spectrum.” As used herein, the term “infrared spectrum” includes wavelengths greater than about 670 nm. As such, the “infrared spectrum” includes near-infrared wavelengths, i.e. those wavelengths adjacent to the “visible spectrum.”

In some embodiments, a graph of absorption versus wavelength is called a spectrum. The spectra can be used to determine the color of a conventional dye (i.e. the spectral response in the visible region) or the characteristics of a detectable moiety (e.g. a detectable moiety's ultra-violet, near-ultraviolet, infrared, or near-infrared characteristics). The wavelength at which the maximum fraction of light is absorbed by a substance is referred to as λmax. Because this wavelength is absorbed to the greatest extent, it is typically referred to as the peak absorbance wavelength. In some embodiments, if radiation of a particular and discrete wavelength is used to illuminate a sample, then there may be an absorption of that radiation. At other wavelengths, such absorption will be reduced or not occur. It is this absorption phenomenon which is used to characterize materials, e.g. the conventional dyes and detectable moieties described herein.

The conventional stains (“routine stains” or “special stains”) of the present disclosure utilize one or more conventional dyes, including any of those described herein. In some embodiments, the conventional dyes of the routine stains and special stains have wavelengths detectable “within the visible spectrum,” such as between about 400 nm and about 700 nm. By way of example, FIG. 9 illustrates the absorbance of hematoxylin and eosin stained tonsil tissue, with the human visual response superimposed. FIG. 9 shows that the hematoxylin and eosin stain absorbs across the visible range. FIG. 9 also shows that the absorbances of hematoxylin and eosin decline outside the region of visual response and is greatly reduced below about 450 nm and above about 700 nm. Other non-limiting examples of suitable conventional dyes and their peak absorbance wavelengths (or two peak absorbance wavelengths, if applicable) are described herein.

The present disclosure is also directed to one or more detectable moieties which are used in conjunction with the one or more conventional dyes in staining biological specimens. In some embodiments, the detectable moieties of the present disclosure generally have peak absorbance wavelengths of less than about 430 nm. In other embodiments, the detectable moieties of the present disclosure generally have peak absorbance wavelengths of greater than about 670 nm. As such, the detectable moieties of the present disclosure have wavelengths within either the “ultraviolet spectrum” or the “infrared spectrum.” Collectively, those detectable moieties having wavelengths of less than about 430 nm or greater than about 670 nm are referred to herein as detectable “outside the visible spectrum.”

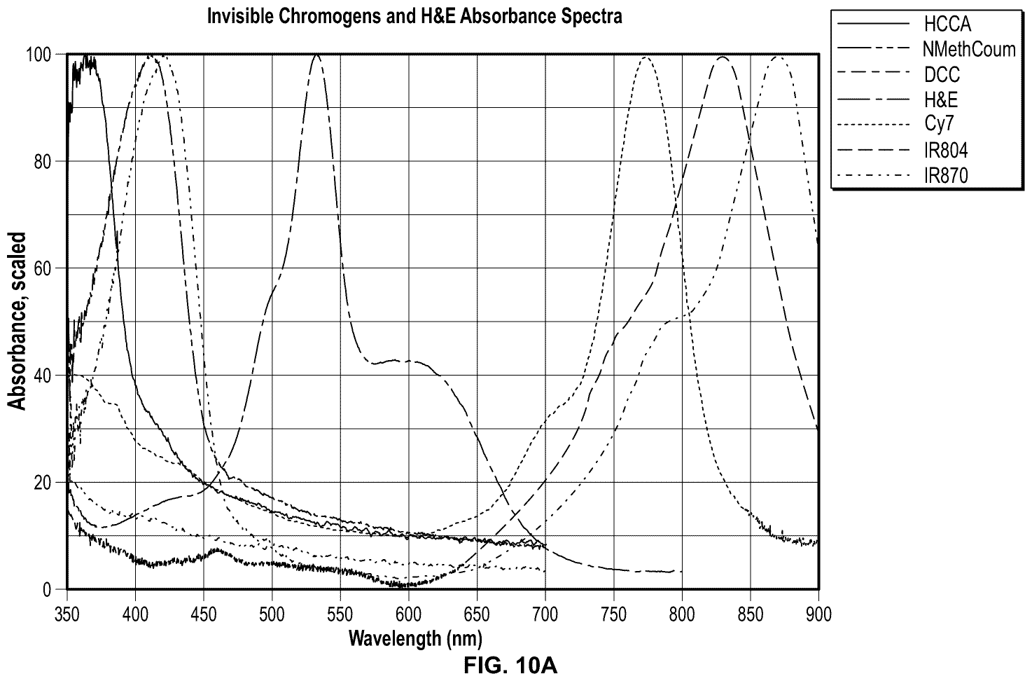

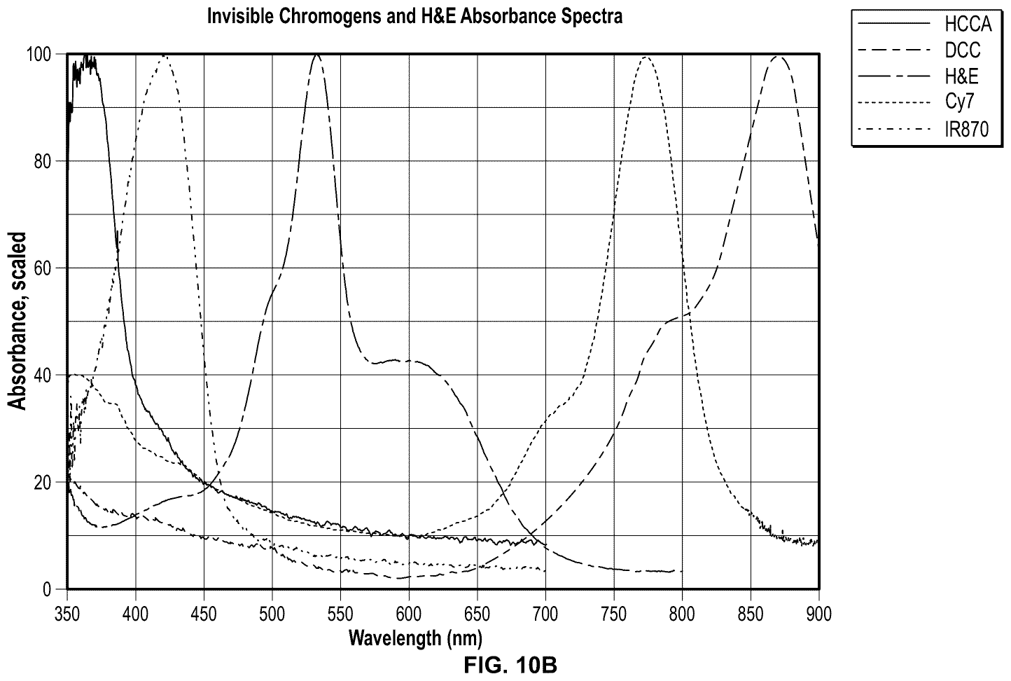

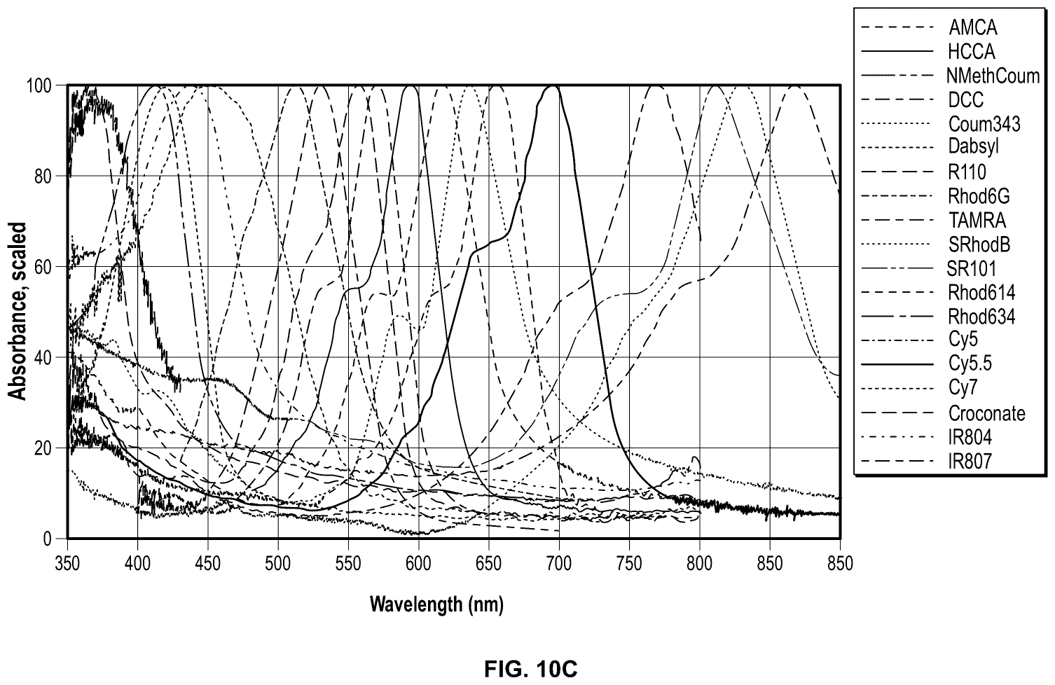

For example, FIG. 10 illustrates an “HCCA” detectable moiety within the ultraviolet spectrum (having a peak absorbance wavelength of 365 nm), and “Cy7” and “IR804” detectable moieties (having peak absorbance wavelengths of 774 nm and 828 nm, respectively) within the infrared spectrum. HCCA, Cy7, and IR804 are hence “outside the visible spectrum.”

When detectable moieties that are detectable “outside the visible spectrum” are selected for use in combination with conventional dyes that are detectable “within the visible spectrum,” it does not mean that there cannot be overlap between the visible spectrum and ultraviolet spectrum (e.g. those wavelengths between 370 nm and 430 nm) or between the visible spectrum and the infrared spectrum (e.g. those wavelengths between 670 nm and 730 nm) as noted above. For instance, a first detectable moiety may be selected that has a peak absorbance wavelength of about 430 nm and a first conventional dye may be selected that has a peak absorbance wavelength of about 400 nm, provided that there is sufficient separation (e.g. 20 nm) between the peak absorbance wavelength of the first detectable moiety and the peak absorbance wavelength of the first conventional dye such that signals from the first detectable moiety and the first conventional dye may be independently detected. This is also further illustrated in FIG. 10 which also illustrates “NMethCou” and “DCC” detectable moieties which have peak absorbance wavelengths adjacent to the visible spectrum, such as between 410 nm and 420 nm and thus are “outside the visible spectrum.”

In some embodiments, the invisible chromogens of the present disclosure have a weak, non-zero absorbance in an otherwise visible part of the spectrum (e.g. between 400 nm to 450 nm). When these invisible chromogens are deposited in a sufficient amount, e.g. 300 μm, they may provide a visible (and thus detectable) signal. For example, and with reference to FIG. 10, when DCC is deposited on a sample it has a maximum absorbance peak at about 420 nm, but has residual absorbance between 450 nm and 500 nm that may render the chromogen visible but only when deposited in high amounts (e.g. 300 μm). Likewise, and again with reference to FIG. 10, when IR804 is deposited it has a maximum absorbance peak at about 770 nm. IR804, however, has a comparatively lessened absorbance below 700 nm (e.g. between about 650 nm to about 700 nm), which may be visible if high enough concentrations of the invisible chromogen are deposited on the sample.

Conventional Stains

Staining with one or more conventional stains is used to highlight important features of a biological specimen, including tissues and cells, as well as to enhance the tissue contrast. By coloring otherwise transparent tissue sections, these conventional stains allow pathologists and/or researchers to view, under a microscope, tissue morphology or to look for the presence or prevalence of particular cell types, structures or even microorganisms, such as bacteria. Hematoxylin is a basic dye that is commonly used in this process and stains the nuclei giving it a “bluish color” while eosin stains the cell's cytoplasm and connective tissue giving it a “pinkish stain” (see “routine stains,” herein). There are several other staining techniques used for particular cells and components (see “special stains,” herein).

In histopathology, the term “routine staining” refers to the hematoxylin and eosin stain (H&E) that is used “routinely” with all tissue specimens to reveal the underlying tissue structures and conditions. The term “special stains” has long been used to refer to a large number of alternative staining techniques that are used when staining with H&E does not provide all the information the pathologist or researcher needs.

Routine Stains

Hematoxylin and Eosin (H&E) stain is the universal “routine” stain which has been used for over 100 years throughout the world. Simply put, the hematoxylin stains the nuclei blue and the eosin stains the cytoplasm pink. The H&E stain enables the pathologist to visualize the general morphology of the tissue, allowing for a diagnosis and prognosis of many histopathological conditions. With an H&E-stained slide, the pathologist can see most diseases, inflammation (both acute and chronic), mitosis, bacterial infections, necrosis, fibrosis, pigments and accumulations of proteins.

In some embodiments, the conventional stain is hematoxylin. In other embodiments, the conventional stain is eosin. In yet other embodiments, the convention stain includes both hematoxylin and eosin. In some embodiments, the conventional statin is Eosin B (C.I. 45400), Eosin Y (C.I. 45380), Erythrosin (C.I. 45430), and/or Ethyl eosin (C.I. 45386).

On tissue Hematoxylin shows a broad max approximately at 600 but ranging from about 590 to 620 nm depending on conditions such as ‘bluing’ treatment and probably tissue to some extent. On tissue Eosin has an absorbance maximum of about 533 nm. An H&E spectrum recorded on tonsil tissue is illustrated in FIGS. 9 and 10.

Special Stains

Special stains belong to a diverse family of slide-based stains that rely on basic chemical reactions for microscopic visualization and general identification of various tissues, structures, cells, organelles, carbohydrates, minerals and microorganisms. Special stains are “special” because they are not routine. A “special stain” refers to any chemically-based stain useful for histological analysis that is not an immunohistochemical stain, an in-situ hybridization stain, or a “routine stain.” Special stains are chemically-based stains that have been developed in response to difficult to stain tissue types, unusual diseases, infectious diseases or other non-typical situations affecting the tissue. In some embodiments, special stains stain are used to identify and demonstrate particular structures and tissues which are not visualized by H&E stains.

In some embodiments, useful applications of special stains include: (1) the determination of DNA and DNA content, (2) the mode of action of drugs, hormones or of potentially toxic food additives, (3) metabolic biochemistry, (4) biochemistry of disease processes, (5) primary sites of many metastatic tumors, (6) identification of non-pigmented metastatic melanomas, (7) detection of early invading tumors, (8) definition of the margins of surgically resected tumors, (9) identification of Barr bodies, (10) staining cells in ways that can be used as a basis for cell separation by appropriate instrumentation (e.g., fluorescence), and/or (11) identification of micro-organisms (e.g., Cryptococcus neoformans, Helicobacter pylori).
Van Gieson—The van Gieson stain is a stain used to highlight the difference between collagen and other connective tissue, such as muscle tissues. The van Gieson stain is often used to identify the characteristic arrangement of fibers in different types of tumors. The van Gieson stain uses a mixture of picric acid and acid fuchsin (a trisulfonated pararosanaline, absorbance maximum approximately 544 nm) to penetrate the tissue sample, causing collagen to become red. Surrounding muscle tissues and blood cells are stained yellow. In some embodiments, the acid fuchsin has a first peak absorbance wavelength ranging from between about 520 nm to about 570 nm.
Toluidine blue—Toluidine blue is a type of metachromatic dye that stains acidic tissues (absorbance maximum approximately 628 nm). Toluidine blue is particularly attracted to nucleic acids and is therefore used to stain tissues with high concentrations of DNA and RNA. When in contact with Toluidine Blue, nucleic acids become blue in color.
Alcian blue—Alcian blue is a phthalocyanine dye which provides specificity for substances such as glycosaminoglycans and acid mucins (Alcian blue 8G absorbance maximum 615 nm in water) and causes acid mucins and mucosubstances to appear blue, and nuclei to appear reddish pink when counterstain neutral red is used. Alcian blue dyes are water soluble and appear blue as they contain copper. They attach to sulfate and carboxylated acid mucopolysaccharides and glycoproteins, and dye binding is purely electrostatic. This staining is performed to ascertain mucoid degeneration and to identify acid mucins which are released by various connective and epithelial tissue tumors. Nuclear fast red stain (C.I. 60760), having an absorbance maxima ranging from between 505 nm and 535 nm, may be used as a counterstain. Nuclear fast red stain combines nuclear fast red dye with an aluminum sulfate mordant to selectively stain nuclear chromatin red and provide nonspecific background tissue staining in shades of pink.
Giemsa—This is a blood stain that can be used histopathologically for staining of chromatin and nuclear membranes. It stains human and pathogenic cells differently, therefore, it is used in the diagnosis of many diseases as it stains human cells purple, and bacterial cells pink, so that they may be differentiated. It is also used to stain blood cells, so that their composition and structure may be observed. Nuclei are stained purple, and cytoplasm is stained blue to pale pink, depending on cell type. The Giemsa stain differentiates the granules of different blood cells by staining them different colors. Basophils show dark blue granules and eosinophils show orange granules. Typically methylene blue (having an absorbance maximum ranging from between about 656 to about 661 nm), eosin Y (having an absorbance maximum of about 516 nm), and Azure B (having an absorbance maximum of about 639 nm) is applied.
Reticulin—Reticulin staining employs the use of silver impregnation of a section to highlight reticulin fibers (type III collagen). It is mainly used in histopathology of the liver but can also be used to assess abnormalities in the spleen, bone marrow and kidneys. In the liver, both necrosis and cirrhosis cause irregular patterns of reticulin. The stain causes the fibers to be stained black, which contrasts with a paler grey or pink background. During the staining procedure, the tissue must first be oxidized and then sensitized with iron alum before silver is added. Once silver has been added, it must be reduced using formalin so that it becomes visible. The nuclei can also be counterstained red using nuclear-fast red, to make them visible. The primary stain is a silver stain which will have a very broad absorbance, possibly extending into the IR.
Nissl—Nissl staining is used to visualize Nissl substance (clumps of rough endoplasmic reticulum and free polyribosomes), which is found in neurons. This stain distinguishes neurons from glia and the cytoarchitecture of neurons can be well studied with the help of this stain. A loss of Nissl substance can signify abnormalities such as cell injury or degeneration, which in turn can indicate disease. A commonly used dye in this stain is called Cresyl Echt Violet Acetate (having an absorbance maximum ranging from between about 596 to about 601 nm), which is mixed in a solution with distilled water. This stains Nissl substance a dark blue or dark purple color.
Orcein—The orcein stain is used to identify the inclusion bodies of viruses. These are viral particles within human cells and are visible using light microscopy, unlike viruses themselves. The Orcein stain is commonly used to diagnose hepatitis B, which causes inclusion body formation in hepatocytes. Orcein (having an absorbance maximum ranging from between about 575 nm) is composed of a mixture of amino- and hydroxyphenoxazone compounds. The result of the stain is that inclusion bodies are stained a dark brown-purple color. Proteins that are associated with copper also become stain dark purple.
Sudan black B— Sudan black B (having an absorbance maximum of about 598 nm, with a shoulder of about 415 nm) is a non-ionic, hydrophobic dye used to identify lipids and lipofuscins. Lipofuscins are age pigments occurring in older people in permanent cells like neurons and heart cells. Lipofuscin results from a build of lysosomes that have absorbed indigestible parts of cells. Sudan Black B stains lipofuscins black. Sudan black B is commonly used to stain lipids and fats, hence the fact it stains lipofuscins is important. Sudan black B can also stain red blood cells black as well.
Masson's trichrome—Trichrome stains are mixture of three dyes used to differentiate the muscles, collagen fibers, fibrin and erythrocytes in connective tissue. One of the three dyes is usually nuclear stain and the other two dyes mainly differentiate collagen and muscle fibers. Three different dyes in this stain have different sized molecules, which penetrate tissues differently. Where larger molecules can penetrate, smaller ones are displaced. First, an acidic dye such as Biebrich scarlet (having an absorbance maximum of about 505 nm) is used, followed by phosphotungstic and phosphomolybdic acid, and finally a fiber stain such as Light Green (having absorbance maxima of about 422 nm and about 630 nm). Weigert's iron hematoxylin is also used, but as a fixative at the start of the procedure. After the stain has been carried out, nuclei appear blackish or blue, muscle and fibrin appear red, and collagen appears green.
Mallory's trichrome—Mallory's trichrome differentiates between collagen and muscle fibers. Mallory's trichrome includes three dyes, the first one is diluted acid fuchsin (having an absorbance maximum of about 546 nm), the second is diluted phosphomolybdic acid and the third is a mixture of orange G (having an absorbance maximum of about 475 nm), methyl blue (aniline blue; having an absorbance maximum of about 600 nm), oxalic acid and distilled water. At the end of the procedure, nuclei and muscle cells appear red, collagen appears blue, and erythrocytes become orange.
Azan trichrome—The Azan trichrome stain is used to stain muscle and collagen and can be used to differentiate between muscle and collagen tissue, as well as to identify diseases such as liver disorders. Azan trichrome distinguishes cells from extracellular components and stains muscle fibers red, cartilage and bone matrix blue. Like the Mallory phosphomolybdic acid orange G (having an absorbance maximum of about 475 nm) and aniline blue (having an absorbance maximum of about 600 nm) solutions are used, however instead of using acid fuchsin to stain nuclei, it uses a dye called azocarmine (having an absorbance maximum B of about 516 nm, and G of about 511 nm), which is combined with acetic acid and distilled water. Aniline blue is used as a counterstain to azocarmine to dye the nuclei. The procedure results in red nuclei, orange muscle cells, and blue collagen, allowing them to be differentiated under the microscope.
Cason's trichrome—This stain is used to differentiate collagen. Therefore its applications involve the diagnosis of disorders to do with collagen abnormalities. It stains nuclei and cytoplasm red, collagen blue and erythrocytes orange. The stain used is a mixture of dyes, comprising orange G (having an absorbance maximum of about 475 nm), acid fuchsin (having an absorbance maximum of about 546 nm), aniline blue (having an absorbance maximum of about 600 nm), phosphotungstic acid and distilled water.
PAS (Periodic acid Schiff)—This stain colors glycogen, and is therefore used to look at membranes, mucosubstances as well as the presence of fungus. The process of PAS staining usually involves two steps, the first one is the oxidation reaction with periodic acid leading to the formation of aldehydes, the second step is the demonstration of these aldehydes with the help of Schiff s reagent. Fuchsin dye in Schiff reagent gives a range of colors from magenta to purple. The staining process uses periodic acid, hematoxylin, and Schiff's reagent (complex, reaction product with DNA having an absorbance maximum of about 628 nm) which comprises basic fuchsin (having an absorbance maximum between about 547 to about 552 nm) and sodium metabisulfite combined with distilled water and hydrochloric acid. The stain cause nuclei to become blue, and glycogen and fungi to become magenta in color. PAS is useful in a number of diagnostic applications. For example, it can be used to diagnose glycogen storage disease, certain sarcomas and carcinomas, as well as fungal infections.
Weigert's resorcin fuchsin (Weigert's elastic)—This type of stain is used to color elastic fibers. It causes them to be stained blue-black, nuclei to become light blue-black, collagen to become pink or red, and other tissues to become yellow. The solution includes basic fuchsin (having an absorbance maximum of between about 547 to about 552 nm), which produces a complex that attaches to elastic fibers, causing them to become stained. Weigert's Stain solution is also comprised of resorcin, ferric chloride, ethanol, distilled water and hydrochloric acid. Hematoxylin and van Gieson stain are also used as counterstains.
Wright and Wright Giemsa stain—The Wright and Wright Giemsa stains are polychromatic stains because they contain eosin and methylene blue. Giemsa stain additionally contains methylene blue azure and intensifies the nuclear features. The eosin Y is then used to stain cell cytoplasm orange. Both of them are used to stain peripheral blood smear and bone marrow smears. They are used to look at cells as well as their morphology, aiding in the diagnosis of infections and blood diseases such as leukemia.
Aldehyde fuchsin—This stain elastic fibers and beta cell granules in the pancreas. It is also highly selective with few other high affinity basophilic sites, like mast cell granules and cartilage matrix. Elastic tissue fibers become stained a bluish-purple color, as do beta cell granules and sulphated mucins. Aldehyde fuchsin solution contains a mixture of basic fuchsin (having an absorbance maximum of between about 547 to about 552 nm), about 70% ethanol, concentrated hydrochloric acid, and paraldehyde. Aldehyde fuchsin is commonly used in combination with alcian blue.
Acid Fast— A differential stain used to identify acid-fast bacterial organisms, such as the members of the generaus Mycobacterium and Nocardia. Particularly important for in the diagnosis of tuberculosis (Carbol fuchsin and as a primary stain and methylene blue as a counterstain).

In some embodiments, the conventional dye is Acid fuchsin (C.I. 42685; absorbance maximum 546 nm), Alcian blue 8 GX (C.I. 74240; absorbance maximum 615 nm), Alizarin red S (C.I. 58005; absorbance maximum 556 and 596 nm), Auramine O (C.I. 41000; absorbance maximum 370 and 432 nm), Azocarmine B (C.I. 50090; absorbance maximum 516 nm), Azocarmine G (C.I. 50085; absorbance maximum 511 nm), Azure A (C.I. 52005; similar absorbance to Azure B), Azure B (C.I. 52010; absorbance maximum 639 nm), Basic fuchsine (C.I. 42510; absorbance maximum 547-552 nm), Bismarck brown Y (C.I. 21000; absorbance maximum 643 nm), Brilliant cresyl blue (C.I. 51010; absorbance maximum 622 nm), Carmine (C.I. 75470; absorbance maximum protonated 490-495, increasing in base and when combined with metal salts), Chlorazol black E (C.I. 30235; absorbance maximum 500-504 nm and 574-602 nm), Congo red (C.I. 22120; absorbance maximum 497 nm), Cresyl violet (absorbance maximum 596-601 nm), Crystal violet (C.I. 42555; absorbance maximum 590 nm), Darrow red (absorbance maximum 502 nm), Ethyl green (C.I. 42590; absorbance maximum 635 nm 420 nm), Fast green F C F (C.I. 42053; absorbance maximum 624 nm, pH dependent), Giemsa Stain (mixture of impure azure B, methylene blue and eosin Y), Indigo carmine (C.I. 73015; absorbance maximum 608 nm), Janus green B (C.I. 11050; absorbance maximum 630 nm), Jenner stain 1899, Light green SF (C.I. 42095; absorbance maximum 422 and 630 nm), Malachite green (C.I. 42000; absorbance maximum 614 and 425 nm), Martius yellow (C.I. 10315; absorbance maximum 420-432 nm), Methyl orange (C.I. 13025; absorbance maximum 507 nm), Methyl violet 2B (C.I. 42535; absorbance maximum 583-587 nm), Methylene blue (C.I. 52015; absorbance maximum 656-661 nm), Methylene violet (Bernthsen), (C.I. 52041; absorbance maximum 580-601 nm), Neutral red (C.I. 50040; absorbance maximum 454, 529, 541 nm depending upon pH and solvent), Nigrosin (C.I. 50420; absorbance maximum 570-580 nm), Nile blue A (C.I. 51180; absorbance maximum 633-660 nm), Nuclear fast red (C.I. 60760; absorbance maximum 535 and 505 nm), Oil Red O (C.I. 26125; absorbance maximum 518 and 359 nm), Orange G (C.I. 16230; absorbance maximum 475 nm), Orange II (C.I. 15510; absorbance maximum 483 nm), Orcein (absorbance maximum 575-590 pH dependent, Pararosaniline (C.I. 42500; absorbance maximum 545 nm), Phloxin B (C.I. 45410; absorbance maximum 548 and 510 nm), Pyronine B (C.I. 45010; closely related to Pyronine Y), Pyronine Y (C.I. 45005; absorbance maximum 546-549 nm), Resazurin (absorbance maximum 598 nm in water, 478 in methanol), Rose Bengal (C.I. 45435; absorbance maximum 546 nm), Safranine O (C.I. 50240; absorbance maximum 530 nm), Sudan black B (C.I. 26150; absorbance maximum 598 and 415 nm nm), Sudan III (C.I. 26100; absorbance maximum 503-507 and 503 nm), Sudan IV (C.I. 26105; absorbance maximum 520 nm), Tetrachrome stain (MacNeal), Thionine (C.I. 52000; absorbance maximum 598-602 nm), Toluidine blue (C.I. 52040; absorbance maximum 626-630 nm), Weigert's resorcin fuchsine (absorbance maximum 508 nm), Wright stain, and any combination thereof. In each of these examples, “C.I.” refers to Color Index™. The Color Index™ describes a commercial product by its recognized usage class, its hue and a serial number (which simply reflects the chronological order in which related colorant types have been registered with the Color Index). This definition enables a particular product to be classified along with other products whose essential colorant is of the same chemical constitution and in which that essential colorant results from a single chemical reaction or a series of reactions.

In some embodiments, the visible dye is congo red, Biebrich scarlet-acid fuchsin, Carbol-fuchsin, Gold chloride, Malachite Green, Methyl Green-Pyronin, Phosphomolybdic acid, Safranin O, silver stain, and any combination thereof.

Biomarkers and Non-Limiting Examples of Biomarkers which May be Labeled

In some embodiments, the one or more targets within the biological specimen are biomarkers. The term “biomarker” as used herein refers to an indicator, e.g., predictive, diagnostic, and/or prognostic, which can be detected in a biological specimen, for example, PD-L1. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. Included as illustrative embodiments are antigens, epitopes, cellular proteins, transmembrane proteins, and DNA or RNA sequences. The Her-2/neu gene and protein are both illustrative embodiments of biomarkers.

As noted above, the biomarker targets can be nucleic acid sequences or proteins. Throughout this disclosure when reference is made to a target biomarker protein it is understood that the nucleic acid sequences associated with that protein can also be used as a biomarker target. In some embodiments, the biomarker target is a protein or nucleic acid molecule from a pathogen, such as a virus, bacteria, or intracellular parasite, such as from a viral genome. For example, a biomarker target protein may be produced from a target nucleic acid sequence associated with (e.g., correlated with, causally implicated in, etc.) a disease.

A biomarker target nucleic acid sequence can vary substantially in size. Without limitation, the nucleic acid sequence can have a variable number of nucleic acid residues. For example, a biomarker target nucleic acid sequence can have at least about 10 nucleic acid residues, or at least about 20, 30, 50, 100, 150, 500, 1000 residues. Similarly, a biomarker target polypeptide can vary substantially in size. Without limitation, the biomarker target polypeptide will include at least one epitope that binds to a peptide specific antibody, or fragment thereof. In some embodiments that polypeptide can include at least two epitopes that bind to a peptide specific antibody, or fragment thereof.

In specific, non-limiting embodiments, a biomarker target protein is produced by a target nucleic acid sequence (e.g., genomic target nucleic acid sequence) associated with a neoplasm (for example, a cancer). Numerous chromosome abnormalities (including translocations and other rearrangements, amplification or deletion) have been identified in neoplastic cells, especially in cancer cells, such as B cell and T cell leukemias, lymphomas, breast cancer, colon cancer, neurological cancers and the like. Therefore, in some embodiments, at least a portion of the biomarker target molecule is produced by a nucleic acid sequence (e.g., genomic target nucleic acid sequence) amplified or deleted in at least a subset of cells in a sample.

Oncogenes are known to be responsible for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes FPC nucleic acid molecules generated from a target nucleic acid sequence that extends distally from the SYT gene, and the second probe includes FPC nucleic acid generated from a target nucleic acid sequence that extends 3′ or proximal to the SYT gene. When probes corresponding to these target nucleic acid sequences (e.g., genomic target nucleic acid sequences) are used in an in situ hybridization procedure, normal cells, which lack a t(18q11.2) in the SYT gene region, exhibit two fusions (generated by the two labels in close proximity) signals, reflecting the two intact copies of SYT. Abnormal cells with a t(18q11.2) exhibit a single fusion signal.

In other embodiments, a biomarker target protein produced from a nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1) are characteristic molecular features of certain types of solid tumors of the central nervous system.

The aforementioned embodiments are provided solely for purpose of illustration and are not intended to be limiting. Numerous other cytogenetic abnormalities that correlate with neoplastic transformation and/or growth are known to those of ordinary skill in the art. Biomarker target proteins that are produced by nucleic acid sequences (e.g., genomic target nucleic acid sequences), which have been correlated with neoplastic transformation and which are useful in the disclosed methods, also include the EGFR gene (7p12; e.g., GENBANK™ Accession No. NC-000007, nucleotides 55054219-55242525), the C-MYC gene (8q24.21; e.g., GENBANK™ Accession No. NC-000008, nucleotides 128817498-128822856), D5S271 (5p15.2), lipoprotein lipase (LPL) gene (8p22; e.g., GENBANK™ Accession No. NC-000008, nucleotides 19841058-19869049), RB 1 (13q14; e.g., GENBANK™ Accession No. NC-000013, nucleotides 47775912-47954023), p53 (17p13.1; e.g., GENBANK™ Accession No. NC-000017, complement, nucleotides 7512464-7531642)), N-MYC (2p24; e.g., GENBANK™ Accession No. NC-000002, complement, nucleotides 151835231-151854620), CHOP (12q13; e.g., GENBANK™ Accession No. NC-000012, complement, nucleotides 56196638-56200567), FUS (16p11.2; e.g., GENBANK™ Accession No. NC-000016, nucleotides 31098954-31110601), FKHR (13p14; e.g., GENBANK™ Accession No. NC-000013, complement, nucleotides 40027817-40138734), as well as, for example: ALK (2p23; e.g., GENBANK™ Accession No. NC-000002, complement, nucleotides 29269144-29997936), Ig heavy chain, CCND1 (11q13; e.g., GENBANK™ Accession No. NC-000011, nucleotides 69165054.69178423), BCL2 (18q21.3; e.g., GENBANK™ Accession No. NC-000018, complement, nucleotides 58941559-59137593), BCL6 (3q27; e.g., GENBANK™ Accession No. NC-000003, complement, nucleotides 188921859-188946169), MALF1, AP1 (1p32-p31; e.g., GENBANK™ Accession No. NC-000001, complement, nucleotides 59019051-59022373), TOP2A (17q21-q22; e.g., GENBANK™ Accession No. NC-000017, complement, nucleotides 35798321-35827695), TMPRSS (21q22.3; e.g., GENBANK™ Accession No. NC-000021, complement, nucleotides 41758351-41801948), ERG (21q22.3; e.g., GENBANK™ Accession No. NC-000021, complement, nucleotides 38675671-38955488); ETV1 (7p21.3; e.g., GENBANK™ Accession No. NC-000007, complement, nucleotides 13897379-13995289), EWS (22q12.2; e.g., GENBANK™ Accession No. NC-000022, nucleotides 27994271-28026505); Fill (11q24.1-q24.3; e.g., GENBANK™ Accession No. NC-000011, nucleotides 128069199-128187521), PAX3 (2q35-q37; e.g., GENBANK™ Accession No. NC-000002, complement, nucleotides 222772851-222871944), PAX7 (1p36.2-p36.12; e.g., GENBANK™ Accession No. NC-000001, nucleotides 18830087-18935219), PTEN (10q23.3; e.g., GENBANK™ Accession No. NC-000010, nucleotides 89613175-89716382), AKT2 (19q13.1-q13.2; e.g., GENBANK™ Accession No. NC-000019, complement, nucleotides 45431556-45483036), MYCL1 (1p34.2; e.g., GENBANK™ Accession No. NC-000001, complement, nucleotides 40133685-40140274), REL (2p13-p12; e.g., GENBANK™ Accession No. NC-000002, nucleotides 60962256-61003682) and CSF1R (5q33-q35; e.g., GENBANK™ Accession No. NC-000005, complement, nucleotides 149413051-149473128).

In other embodiments, a biomarker target protein is selected from a virus or other microorganism associated with a disease or condition. Detection of the virus- or microorganism-derived target nucleic acid sequence (e.g., genomic target nucleic acid sequence) in a cell or biological specimen is indicative of the presence of the organism. For example, the biomarker target peptide, polypeptide or protein can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some embodiments, the biomarker target protein is produced from a nucleic acid sequence (e.g., genomic target nucleic acid sequence) from a viral genome. Exemplary viruses and corresponding genomic sequences (GENBANK™ RefSeq Accession No. in parentheses) include human adenovirus A (NC-001460), human adenovirus B (NC-004001), human adenovirus C(NC-001405), human adenovirus D (NC-002067), human adenovirus E (NC-003266), human adenovirus F (NC-001454), human astrovirus (NC-001943), human BK polyomavirus (V01109; GI:60851) human bocavirus (NC-007455), human coronavirus 229E (NC-002645), human coronavirus HKU1 (NC-006577), human coronavirus NL63 (NC-005831), human coronavirus 0C43 (NC-005147), human enterovirus A (NC-001612), human enterovirus B (NC-001472), human enterovirus C(NC-001428), human enterovirus D (NC-001430), human erythrovirus V9 (NC-004295), human foamy virus (NC-001736), human herpesvirus 1 (Herpes simplex virus type 1) (NC-001806), human herpesvirus 2 (Herpes simplex virus type 2) (NC-001798), human herpesvirus 3 (Varicella zoster virus) (NC-001348), human herpesvirus 4 type 1 (Epstein-Barr virus type 1) (NC-007605), human herpesvirus 4 type 2 (Epstein-Barr virus type 2) (NC-009334), human herpesvirus 5 strain AD 169 (NC-001347), human herpesvirus 5 strain Merlin Strain (NC-006273), human herpesvirus 6A (NC-001664), human herpesvirus 6B (NC-000898), human herpesvirus 7 (NC-001716), human herpesvirus 8 type M (NC-003409), human herpesvirus 8 type P (NC-009333), human immunodeficiency virus 1 (NC-001802), human immunodeficiency virus 2 (NC-001722), human metapneumovirus (NC-004148), human papillomavirus-1 (NC-001356), human papillomavirus-18 (NC-001357), human papillomavirus-2 (NC-001352), human papillomavirus-54 (NC-001676), human papillomavirus-61 (NC-001694), human papillomavirus-cand90 (NC-004104), human papillomavirus RTRX7 (NC-004761), human papillomavirus type 10 (NC-001576), human papillomavirus type 101 (NC-008189), human papillomavirus type 103 (NC-008188), human papillomavirus type 107 (NC-009239), human papillomavirus type 16 (NC-001526), human papillomavirus type 24 (NC-001683), human papillomavirus type 26 (NC-001583), human papillomavirus type 32 (NC-001586), human papillomavirus type 34 (NC-001587), human papillomavirus type 4 (NC-001457), human papillomavirus type 41 (NC-001354), human papillomavirus type 48 (NC-001690), human papillomavirus type 49 (NC-001591), human papillomavirus type 5 (NC-001531), human papillomavirus type 50 (NC-001691), human papillomavirus type 53 (NC-001593), human papillomavirus type 60 (NC-001693), human papillomavirus type 63 (NC-001458), human papillomavirus type 6b (NC-001355), human papillomavirus type 7 (NC-001595), human papillomavirus type 71 (NC-002644), human papillomavirus type 9 (NC-001596), human papillomavirus type 92 (NC-004500), human papillomavirus type 96 (NC-005134), human parainfluenza virus 1 (NC-003461), human parainfluenza virus 2 (NC-003443), human parainfluenza virus 3 (NC-001796), human parechovirus (NC-001897), human parvovirus 4 (NC-007018), human parvovirus B19 (NC-000883), human respiratory syncytial virus (NC-001781), human rhinovirus A (NC-001617), human rhinovirus B (NC-001490), human spumaretrovirus (NC-001795), human T-lymphotropic virus 1 (NC-001436), human T-lymphotropic virus 2 (NC-001488).

In certain embodiments, the biomarker target protein is produced from a nucleic acid sequence (e.g., genomic target nucleic acid sequence) from an oncogenic virus, such as Epstein-Barr Virus (EBV) or a Human Papilloma Virus (HPV, e.g., HPV16, HPV18). In other embodiments, the target protein produced from a nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., S ARS virus), an Adenovirus, a Polyomavirus, a Cytomegalovirus (CMV), or a Herpes Simplex Virus (HSV).

### Detectable Moieties

The presently disclosed methods utilize one or more detectable moieties. In some embodiments, the detectable moieties are a component of a detectable conjugate. In some embodiments, the detectable conjugates which may be used in the presently disclosed methods include the detectable moiety and one of a tyramide moiety (or a derivative or analog thereof), a quinone methide moiety (or a derivative or analog thereof), or a functional group capable of participating in a “click chemistry” reaction (see also U.S. Pat. No. 10,041,950, and in U.S. Publication Nos. 2019/0204330, 2017/0089911, and 2019/0187130, the disclosures of which are hereby incorporated by reference herein in their entireties). In other embodiments, the detectable conjugates which may be used in the presently disclosed methods include the detectable moiety and one of a hapten, an enzyme, or an antibody.

In some embodiments, suitable detectable moieties may be characterized according to a full width of an absorbance peak at the half maximum absorbance, referred to herein as FWHM (“full-width half-max”). FWHM is an expression of the extent of function given by the difference between the two extreme values of the independent variable at which the dependent variable is equal to half of its maximum value. In other words, it is the width of a spectrum curve measured between those points on the y-axis which are half the maximum amplitude. It is given by the distance between points on the curve at which the function reaches half its maximum value. Essentially, FWHM is a parameter commonly used to describe the width of a “bump” on a curve or function. In some embodiments, while an absorbance maximum (λₘₐₓ) may describe the wavelength of maximum absorption of a detectable moiety, the FWHM describes the breadth of the spectral absorbance.

In some embodiments, the detectable moieties of the present disclosure have a narrow FWHM. In some embodiments, the FWHM of the detectable moieties have a FWHM which is 40% less than a FWHM of a dye, chromophore or fluorophore (e.g. dabsyl, hematoxylin); 50% less than a FWHM of a dye, chromophore or fluorophore; 55% less than a FWHM of a dye, chromophore or fluorophore; 65% less than a FWHM of a dye, chromophore or fluorophore; 70% less than a FWHM of a dye, chromophore or fluorophore; 75% less than a FWHM of a dye, chromophore or fluorophore; 80% less than the FWHM of a dye, chromophore or fluorophore; 85% less than a FWHM of a dye, chromophore or fluorophore; 90% less than a FWHM of a chromophore or fluorophore; or 95% less than a FWHM of a dye, chromophore or fluorophore.

In some embodiments, the detectable moieties have an absorbance peak with FWHM of between about 15 nm and about 200 nm. In some embodiments, the detectable moieties have an absorbance peak with FWHM of between about 15 nm and about 150 nm. In some embodiments, the detectable moieties have an absorbance peak with FWHM of between about 15 nm and about 100 nm. In some embodiments, the detectable moieties have an absorbance peak with FWHM of between about 15 nm and about 70 nm. In some embodiments the detectable moieties have has an absorbance peak with FWHM of between about 15 nm and about 50 nm. In some embodiments the detectable moieties have an absorbance peak with FWHM of between about 15 nm and about 40 nm.

In some embodiments, the detectable moieties have an absorbance peak with FWHM of between about 20 nm and about 200 nm. In some embodiments, the detectable moieties have an absorbance peak with FWHM of between about 20 nm and about 150 nm. In some embodiments, the detectable moieties have an absorbance peak with FWHM of between about 20 nm and about 100 nm. In some embodiments, the detectable moieties have an absorbance peak with FWHM of between about 20 nm and about 70 nm. In some embodiments the detectable moieties have has an absorbance peak with FWHM of between about 20 nm and about 50 nm.

In some embodiments, the detectable moieties have an absorbance peak with FWHM of between about 30 nm and about 200 nm. In some embodiments, the detectable moieties have an absorbance peak with FWHM of between about 30 nm and about 150 nm. In some embodiments, the detectable moieties have an absorbance peak with FWHM of between about 30 nm and about 100 nm. In some embodiments, the detectable moieties have an absorbance peak with FWHM of between about 30 nm and 70 nm. In some embodiments, the detectable moieties have an absorbance peak with FWHM of between 30 nm and 50 nm.

In some embodiments, the detectable moieties have an absorbance peak with FWHM of between about 40 nm and about 200 nm. In some embodiments, the detectable moieties have an absorbance peak with FWHM of between about 40 nm and about 150 nm. In some embodiments, the detectable moieties have an absorbance peak with FWHM of between about 40 nm and about 100 nm. In some embodiments, the detectable moieties have an absorbance peak with FWHM of between about 40 nm and 70 nm.

In some embodiments, the detectable moieties have an absorbance peak with FWHM of between about 50 nm and about 200 nm. In some embodiments, the detectable moieties have an absorbance peak with FWHM of between about 50 nm and about 150 nm. In some embodiments, the detectable moieties have an absorbance peak with FWHM of between about 50 nm and about 100 nm. In some embodiments, the detectable moieties have an absorbance peak with FWHM of between about 50 nm and 70 nm.

In some embodiments, the detectable moieties have an absorbance peak with FWHM of between about 60 nm and about 200 nm. In some embodiments, the detectable moieties have an absorbance peak with FWHM of between about 60 nm and about 150 nm. In some embodiments, the detectable moieties have an absorbance peak with FWHM of between about 60 nm and about 100 nm.

In some embodiments, the detectable moieties have an absorbance peak with FWHM of between about 70 nm and about 200 nm. In some embodiments, the detectable moieties have an absorbance peak with FWHM of between about 70 nm and about 150 nm. In some embodiments, the detectable moieties have an absorbance peak with FWHM of between about 70 nm and about 100 nm.

In some embodiments, the detectable moieties have an absorbance peak with FWHM of less than about 200 nm. In some embodiments, the detectable moieties have an absorbance peak with FWHM of less than about 190 nm. In some embodiments, the detectable moieties have an absorbance peak with FWHM of less than about 180 nm. In some embodiments, the detectable moieties have an absorbance peak with FWHM of less than about 170 nm. In some embodiments, the detectable moieties have an absorbance peak with FWHM of less than about 160 nm. In some embodiments, the detectable moieties have an absorbance peak with FWHM of less than about 150 nm. In some embodiments, the detectable moieties have an absorbance peak with FWHM of less than about 140 nm. In some embodiments, the detectable moieties have an absorbance peak with FWHM of less than about 130 nm. In some embodiments, the detectable moieties have an absorbance peak with FWHM of less than about 120 nm. In some embodiments, the detectable moieties have an absorbance peak with FWHM of less than about 110 nm. In some embodiments, the detectable moieties have an absorbance peak with FWHM of less than about 100 nm. In some embodiments, the detectable moieties have an absorbance peak with FWHM of less than about 90 nm. In some embodiments, the detectable moieties have an absorbance peak with FWHM of less than about 80 nm. In some embodiments, the detectable moieties have an absorbance peak with FWHM of less than about 70 nm. In some embodiments, the detectable moieties have an absorbance peak with FWHM of less than about 60 nm. In some embodiments, the detectable moieties have an absorbance peak with FWHM of less than about 50 nm.

Detectable Moieties within the Ultraviolet Spectrum

In some embodiments, the detectable moieties have a peak absorbance wavelength within the ultraviolet spectrum. In some embodiments, the detectable moieties have a peak absorbance peak absorbance wavelength of less than about 430 nm. In some embodiments, the detectable moieties have a peak absorbance peak absorbance wavelength of less than about 420 nm. In some embodiments, the detectable moieties have a peak absorbance wavelength of less than about 415 nm. In some embodiments, the detectable moieties have a peak absorbance wavelength of less than about 410 nm. In some embodiments, the detectable moieties have a peak absorbance wavelength of less than about 400 nm. In some embodiments, the detectable moieties have a peak absorbance wavelength of less than about 405 nm. In some embodiments, the detectable moiety of the disclosed compounds has a peak absorbance wavelength of less than about 395 nm. In some embodiments, the detectable moieties have a peak absorbance wavelength of less than about 390 nm. In some embodiments, the detectable moieties have a peak absorbance wavelength of less than about 385 nm. In some embodiments, the detectable moieties have a peak absorbance wavelength of less than about 380 nm. In some embodiments, the detectable moieties have a peak absorbance wavelength of less than about 375 nm. In some embodiments, the detectable moiety of the disclosed compounds has a peak absorbance wavelength of less than about 370 nm. In some embodiments, the detectable moieties have a peak absorbance wavelength ranging from between about 100 nm to about 400 nm, from about 100 nm to about 390 nm, from about 100 nm to about 380 nm, or from about 100 nm to about 370 nm.

In some embodiments, the detectable moieties have a peak absorbance wavelength of less than about 420 nm and a FWHM of less than about 200 nm. In some embodiments, the detectable moieties have a peak absorbance wavelength of less than about 415 nm and a FWHM of less than about 200 nm. In some embodiments, the detectable moieties have a peak absorbance wavelength of less than about 410 nm and a FWHM of less than about 200 nm. In some embodiments, the detectable moieties have a peak absorbance wavelength of less than about 400 nm and a FWHM of less than about 200 nm. In some embodiments, the detectable moieties have a peak absorbance wavelength of less than about 405 nm and a FWHM of less than about 200 nm. In some embodiments, the detectable moiety of the disclosed compounds has a peak absorbance wavelength of less than about 395 nm and a FWHM of less than about 200 nm. In some embodiments, the detectable moieties have a peak absorbance wavelength of less than about 390 nm and a FWHM of less than about 200 nm. In some embodiments, the detectable moieties have a peak absorbance wavelength of less than about 385 nm and a FWHM of less than about 200 nm. In some embodiments, the detectable moieties have a peak absorbance wavelength of less than about 380 nm and a FWHM of less than about 200 nm. In some embodiments, the detectable moieties have a peak absorbance wavelength of less than about 375 nm and a FWHM of less than about 200 nm. In some embodiments, the detectable moiety of the disclosed compounds has a peak absorbance wavelength of less than about 370 nm and a FWHM of less than about 200 nm.

In some embodiments, the detectable moieties have a peak absorbance wavelength of less than about 420 nm and a FWHM of less than about 150 nm. In some embodiments, the detectable moieties have a peak absorbance wavelength of less than about 415 nm and a FWHM of less than about 150 nm. In some embodiments, the detectable moieties have a peak absorbance wavelength of less than about 410 nm and a FWHM of less than about 150 nm. In some embodiments, the detectable moieties have a peak absorbance wavelength of less than about 400 nm and a FWHM of less than about 150 nm. In some embodiments, the detectable moieties have a peak absorbance wavelength of less than about 405 nm and a FWHM of less than about 150 nm. In some embodiments, the detectable moiety of the disclosed compounds has a peak absorbance wavelength of less than about 395 nm and a FWHM of less than about 150 nm. In some embodiments, the detectable moieties have a peak absorbance wavelength of less than about 390 nm and a FWHM of less than about 150 nm. In some embodiments, the detectable moieties have a peak absorbance wavelength of less than about 385 nm and a FWHM of less than about 150 nm. In some embodiments, the detectable moieties have a peak absorbance wavelength of less than about 380 nm and a FWHM of less than about 150 nm. In some embodiments, the detectable moieties have a peak absorbance wavelength of less than about 375 nm and a FWHM of less than about 150 nm. In some embodiments, the detectable moiety of the disclosed compounds has a peak absorbance wavelength of less than about 370 nm and a FWHM of less than about 150 nm.

In some embodiments, the detectable moieties have a peak absorbance wavelength of less than about 420 nm and a FWHM of less than about 100 nm. In some embodiments, the detectable moieties have a peak absorbance wavelength of less than about 415 nm and a FWHM of less than about 100 nm. In some embodiments, the detectable moieties have a peak absorbance wavelength of less than about 410 nm and a FWHM of less than about 100 nm. In some embodiments, the detectable moieties have a peak absorbance wavelength of less than about 400 nm and a FWHM of less than about 100 nm. In some embodiments, the detectable moieties have a peak absorbance wavelength of less than about 405 nm and a FWHM of less than about 100 nm. In some embodiments, the detectable moiety of the disclosed compounds has a peak absorbance wavelength of less than about 395 nm and a FWHM of less than about 100 nm. In some embodiments, the detectable moieties have a peak absorbance wavelength of less than about 390 nm and a FWHM of less than about 100 nm. In some embodiments, the detectable moieties have a peak absorbance wavelength of less than about 385 nm and a FWHM of less than about 100 nm. In some embodiments, the detectable moieties have a peak absorbance wavelength of less than about 380 nm and a FWHM of less than about 100 nm. In some embodiments, the detectable moieties have a peak absorbance wavelength of less than about 375 nm and a FWHM of less than about 100 nm. In some embodiments, the detectable moiety of the disclosed compounds has a peak absorbance wavelength of less than about 370 nm and a FWHM of less than about 100 nm.

In some embodiments, the detectable moieties have a peak absorbance wavelength of less than about 420 nm and a FWHM of less than about 70 nm. In some embodiments, the detectable moieties have a peak absorbance wavelength of less than about 415 nm and a FWHM of less than about 70 nm. In some embodiments, the detectable moieties have a peak absorbance wavelength of less than about 410 nm and a FWHM of less than about 70 nm. In some embodiments, the detectable moieties have a peak absorbance wavelength of less than about 400 nm and a FWHM of less than about 70 nm. In some embodiments, the detectable moieties have a peak absorbance wavelength of less than about 405 nm and a FWHM of less than about 70 nm. In some embodiments, the detectable moiety of the disclosed compounds has a peak absorbance wavelength of less than about 395 nm and a FWHM of less than about 70 nm. In some embodiments, the detectable moieties have a peak absorbance wavelength of less than about 390 nm and a FWHM of less than about 70 nm. In some embodiments, the detectable moieties have a peak absorbance wavelength of less than about 385 nm and a FWHM of less than about 70 nm. In some embodiments, the detectable moieties have a peak absorbance wavelength of less than about 380 nm and a FWHM of less than about 70 nm. In some embodiments, the detectable moieties have a peak absorbance wavelength of less than about 375 nm and a FWHM of less than about 70 nm. In some embodiments, the detectable moiety of the disclosed compounds has a peak absorbance wavelength of less than about 370 nm and a FWHM of less than about 70 nm.

In some embodiments, the detectable moiety includes or is derived from a coumarin (i.e. the detectable moiety includes a coumarin core). Examples of suitable detectable moieties having a coumarin core are described in U.S. Pat. No. 10,041,950, the disclosure of which is hereby incorporated by reference herein in its entirety. In some embodiments, the coumarin core is a coumarinamine core. In some embodiments, the coumarin core is a 7-coumarinamine core. In some embodiments, the coumarin core is a coumarinol core. In some embodiments, the coumarin core is a 7-coumarinol core.

In some embodiments, the coumarin core includes (or is modified to include) one or more electron withdrawing groups (where each electron withdrawing group may be the same or different). In some embodiments, the coumarin core includes (or is modified to include) one electron withdrawing group. In some embodiments, the coumarin core includes (or is modified to include) two electron withdrawing groups. In some embodiments, the coumarin core includes (or is modifying to include) three electron withdrawing groups. In some embodiments, the coumarin core includes (or is modifying to include) three different electron withdrawing groups. In some embodiments, the coumarin core includes (or is modified to include) four electron withdrawing groups. In some embodiments, the one or more electron withdrawing groups have an electronegatively ranging from between about 1.5 to about 3.5 each.

In some embodiments, the coumarin core includes (or is modified to include) one or more electron donating groups (where each electron donating group may be the same or different). In some embodiments, the coumarin core includes (or is modified to include) one electron donating group. In some embodiments, the coumarin core includes (or is modified to include) two electron donating groups. In some embodiments, the coumarin core includes (or is modifying to include) three electron donating groups. In some embodiments, the coumarin core includes (or is modifying to include) three different electron donating groups. In some embodiments, the coumarin core includes (or is modified to include) four electron donating groups. In some embodiments, the one or more electron donating groups have an electronegatively ranging from between about 1.5 to about 3.5 each. In some embodiments, one or more electronic withdrawing and/or donating groups are incorporated to facilitate a shift towards the “red” spectrum or the “blue” spectrum.

In some embodiments, the detectable moieties having the coumarin core have a wavelength ranging from about 300 nm to about 460 nm. In some embodiments, the detectable moieties having the coumarin core have a wavelength ranging from about 320 nm to about 440 nm. In some embodiments, the detectable moieties having the coumarin core have a wavelength ranging from about 340 nm to about 430 nm. These ranges may be altered or shift as more or less electronegative is introduced to the coumarin core.

In some embodiments, the detectable moieties having the coumarin core have a peak absorbance wavelength of about 460 nm+/−10 nm. In some embodiments, the detectable moieties having the coumarin core have a peak absorbance wavelength of about 455+/−10 nm. In some embodiments, the detectable moieties having the coumarin core have a peak absorbance wavelength of about 450 nm+/−10 nm. In some embodiments, the detectable moieties having the coumarin core have a peak absorbance wavelength of about 445 nm+/−10 nm. In some embodiments, the detectable moieties having the coumarin core have a peak absorbance wavelength of about 440 nm+/−10 nm. In some embodiments, the detectable moieties having the coumarin core have a peak absorbance wavelength of about 435 nm+/−10 nm. In some embodiments, the detectable moieties having the coumarin core have a peak absorbance wavelength of about 430 nm+/−10 nm. In some embodiments, the detectable moieties having the coumarin core have a peak absorbance wavelength of about 425 nm+/−10 nm. In some embodiments, the detectable moieties having the coumarin core have a peak absorbance wavelength of about 420 nm+/−10 nm. In some embodiments, the detectable moieties having the coumarin core have a peak absorbance wavelength of about 415 nm+/−10 nm. In some embodiments, the detectable moieties having the coumarin core have a peak absorbance wavelength of about 410 nm+/−10 nm. In some embodiments, the detectable moieties having the coumarin core have a peak absorbance wavelength of about 405 nm+/−10 nm. In some embodiments, the detectable moieties having the coumarin core have a peak absorbance wavelength of about 400 nm+/−10 nm. In some embodiments, the detectable moieties having the coumarin core have a peak absorbance wavelength of about 395 nm+/−10 nm. In some embodiments, the detectable moieties having the coumarin core have a peak absorbance wavelength of about 390 nm+/−10 nm. In some embodiments, the detectable moieties having the coumarin core have a peak absorbance wavelength of about 385 nm+/−10 nm. In some embodiments, the detectable moieties having the coumarin core have a peak absorbance wavelength of about 380 nm+/−10 nm. In some embodiments, the detectable moieties having the coumarin core have a peak absorbance wavelength of about 375 nm+/−10 nm. In some embodiments, the detectable moieties having the coumarin core have a peak absorbance wavelength of about 370 nm+/−10 nm. In some embodiments, the detectable moieties having the coumarin core have a peak absorbance wavelength of about 365 nm+/−10 nm. In some embodiments, the detectable moieties having the coumarin core have a peak absorbance wavelength of about 360 nm+/−10 nm. In some embodiments, the detectable moieties having the coumarin core have a peak absorbance wavelength of about 355 nm+/−10 nm. In some embodiments, the detectable moieties having the coumarin core have a peak absorbance wavelength of about 350 nm+/−10 nm. In some embodiments, the detectable moieties having the coumarin core have a peak absorbance wavelength of about 345 nm+/−10 nm. In some embodiments, the detectable moieties having the coumarin core have a peak absorbance wavelength of about 340 nm+/−10 nm. In some embodiments, the detectable moieties having the coumarin core have a peak absorbance wavelength of about 335 nm+/−10 nm. In some embodiments, the detectable moieties having the coumarin core have a peak absorbance wavelength of about 330 nm+/−10 nm.

In some embodiments, the detectable moieties having the coumarin core have a peak absorbance wavelength of about 460 nm+/−10 nm and a FWHM of less than about 200 nm. In some embodiments, the detectable moieties having the coumarin core have a peak absorbance wavelength of about 455+/−10 nm and a FWHM of less than about 200 nm. In some embodiments, the detectable moieties having the coumarin core have a peak absorbance wavelength of about 450 nm+/−10 nm and a FWHM of less than about 200 nm. In some embodiments, the detectable moieties having the coumarin core have a peak absorbance wavelength of about 445 nm+/−10 nm and a FWHM of less than about 200 nm. In some embodiments, the detectable moieties having the coumarin core have a peak absorbance wavelength of about 440 nm+/−10 nm and a FWHM of less than about 200 nm. In some embodiments, the detectable moieties having the coumarin core have a peak absorbance wavelength of about 435 nm+/−10 nm and a FWHM of less than about 200 nm. In some embodiments, the detectable moieties having the coumarin core have a peak absorbance wavelength of about 430 nm+/−10 nm and a FWHM of less than about 200 nm. In some embodiments, the detectable moieties having the coumarin core have a peak absorbance wavelength of about 425 nm+/−10 nm and a FWHM of less than about 200 nm. In some embodiments, the detectable moieties having the coumarin core have a peak absorbance wavelength of about 420 nm+/−10 nm and a FWHM of less than about 200 nm. In some embodiments, the detectable moieties having the coumarin core have a peak absorbance wavelength of about 415 nm+/−10 nm and a FWHM of less than about 200 nm. In some embodiments, the detectable moieties having the coumarin core have a peak absorbance wavelength of about 410 nm and a FWHM of less than about 200 nm. In some embodiments, the detectable moieties having the coumarin core have a peak absorbance wavelength of about 405 nm+/−10 nm and a FWHM of less than about 200 nm. In some embodiments, the detectable moieties having the coumarin core have a peak absorbance wavelength of about 400 nm+/−10 nm and a FWHM of less than about 200 nm. In some embodiments, the detectable moieties having the coumarin core have a peak absorbance wavelength of about 395 nm+/−10 nm and a FWHM of less than about 200 nm. In some embodiments, the detectable moieties having the coumarin core have a peak absorbance wavelength of about 390 nm+/−10 nm and a FWHM of less than about 200 nm. In some embodiments, the detectable moieties having the coumarin core have a peak absorbance wavelength of about 385 nm+/−10 nm and a FWHM of less than about 200 nm. In some embodiments, the detectable moieties having the coumarin core have a peak absorbance wavelength of about 380 nm+/−10 nm and a FWHM of less than about 200 nm. In some embodiments, the detectable moieties having the coumarin core have a peak absorbance wavelength of about 375 nm+/−10 nm and a FWHM of less than about 200 nm. In some embodiments, the detectable moieties having the coumarin core have a peak absorbance wavelength of about 370 nm+/−10 nm and a FWHM of less than about 200 nm. In some embodiments, the detectable moieties having the coumarin core have a peak absorbance wavelength of about 365 nm+/−10 nm and a FWHM of less than about 200 nm. In some embodiments, the detectable moieties having the coumarin core have a peak absorbance wavelength of about 360 nm+/−10 nm and a FWHM of less than about 200 nm. In some embodiments, the detectable moieties having the coumarin core have a peak absorbance wavelength of about 355 nm+/−10 nm and a FWHM of less than about 200 nm. In some embodiments, the detectable moieties having the coumarin core have a peak absorbance wavelength of about 350 nm+/−10 nm and a FWHM of less than about 200 nm. In some embodiments, the detectable moieties having the coumarin core have a peak absorbance wavelength of about 345 nm+/−10 nm and a FWHM of less than about 200 nm. In some embodiments, the detectable moieties having the coumarin core have a peak absorbance wavelength of about 340 nm+/−10 nm and a FWHM of less than about 200 nm. In some embodiments, the detectable moieties having the coumarin core have a peak absorbance wavelength of about 335 nm+/−10 nm and a FWHM of less than about 200 nm. In some embodiments, the detectable moieties having the coumarin core have a peak absorbance wavelength of about 330 nm+/−10 nm and a FWHM of less than about 200 nm.

In some embodiments, the detectable moieties having the coumarin core have a peak absorbance wavelength of about 460 nm+/−10 nm and a FWHM of less than about 150 nm. In some embodiments, the detectable moieties having the coumarin core have a peak absorbance wavelength of about 455+/−10 nm and a FWHM of less than about 150 nm. In some embodiments, the detectable moieties having the coumarin core have a peak absorbance wavelength of about 450 nm+/−10 nm and a FWHM of less than about 150 nm. In some embodiments, the detectable moieties having the coumarin core have a peak absorbance wavelength of about 445 nm+/−10 nm and a FWHM of less than about 150 nm. In some embodiments, the detectable moieties having the coumarin core have a peak absorbance wavelength of about 440 nm+/−10 nm and a FWHM of less than about 150 nm. In some embodiments, the detectable moieties having the coumarin core have a peak absorbance wavelength of about 435 nm+/−10 nm and a FWHM of less than about 150 nm. In some embodiments, the detectable moieties having the coumarin core have a peak absorbance wavelength of about 430 nm+/−10 nm and a FWHM of less than about 150 nm. In some embodiments, the detectable moieties having the coumarin core have a peak absorbance wavelength of about 425 nm+/−10 nm and a FWHM of less than about 150 nm. In some embodiments, the detectable moieties having the coumarin core have a peak absorbance wavelength of about 420 nm+/−10 nm and a FWHM of less than about 150 nm. In some embodiments, the detectable moieties having the coumarin core have a peak absorbance wavelength of about 415 nm+/−10 nm and a FWHM of less than about 150 nm. In some embodiments, the detectable moieties having the coumarin core have a peak absorbance wavelength of about 410 nm and a FWHM of less than about 150 nm. In some embodiments, the detectable moieties having the coumarin core have a peak absorbance wavelength of about 405 nm+/−10 nm and a FWHM of less than about 150 nm. In some embodiments, the detectable moieties having the coumarin core have a peak absorbance wavelength of about 400 nm+/−10 nm and a FWHM of less than about 150 nm. In some embodiments, the detectable moieties having the coumarin core have a peak absorbance wavelength of about 395 nm+/−10 nm and a FWHM of less than about 150 nm. In some embodiments, the detectable moieties having the coumarin core have a peak absorbance wavelength of about 390 nm+/−10 nm and a FWHM of less than about 150 nm. In some embodiments, the detectable moieties having the coumarin core have a peak absorbance wavelength of about 385 nm+/−10 nm and a FWHM of less than about 150 nm. In some embodiments, the detectable moieties having the coumarin core have a peak absorbance wavelength of about 380 nm+/−10 nm and a FWHM of less than about 150 nm. In some embodiments, the detectable moieties having the coumarin core have a peak absorbance wavelength of about 375 nm+/−10 nm and a FWHM of less than about 150 nm. In some embodiments, the detectable moieties having the coumarin core have a peak absorbance wavelength of about 370 nm+/−10 nm and a FWHM of less than about 150 nm. In some embodiments, the detectable moieties having the coumarin core have a peak absorbance wavelength of about 365 nm+/−10 nm and a FWHM of less than about 150 nm. In some embodiments, the detectable moieties having the coumarin core have a peak absorbance wavelength of about 360 nm+/−10 nm and a FWHM of less than about 150 nm. In some embodiments, the detectable moieties having the coumarin core have a peak absorbance wavelength of about 355 nm+/−10 nm and a FWHM of less than about 150 nm. In some embodiments, the detectable moieties having the coumarin core have a peak absorbance wavelength of about 350 nm+/−10 nm and a FWHM of less than about 150 nm. In some embodiments, the detectable moieties having the coumarin core have a peak absorbance wavelength of about 345 nm+/−10 nm and a FWHM of less than about 150 nm. In some embodiments, the detectable moieties having the coumarin core have a peak absorbance wavelength of about 340 nm+/−10 nm and a FWHM of less than about 150 nm. In some embodiments, the detectable moieties having the coumarin core have a peak absorbance wavelength of about 335 nm+/−10 nm and a FWHM of less than about 150 nm. In some embodiments, the detectable moieties having the coumarin core have a peak absorbance wavelength of about 330 nm+/−10 nm and a FWHM of less than about 150 nm.

In some embodiments, the detectable moieties having the coumarin core have a peak absorbance wavelength of about 460 nm+/−10 nm and a FWHM of less than about 100 nm. In some embodiments, the detectable moieties having the coumarin core have a peak absorbance wavelength of about 455+/−10 nm and a FWHM of less than about 100 nm. In some embodiments, the detectable moieties having the coumarin core have a peak absorbance wavelength of about 450 nm+/−10 nm and a FWHM of less than about 100 nm. In some embodiments, the detectable moieties having the coumarin core have a peak absorbance wavelength of about 445 nm+/−10 nm and a FWHM of less than about 100 nm. In some embodiments, the detectable moieties having the coumarin core have a peak absorbance wavelength of about 440 nm+/−10 nm and a FWHM of less than about 100 nm. In some embodiments, the detectable moieties having the coumarin core have a peak absorbance wavelength of about 435 nm+/−10 nm and a FWHM of less than about 100 nm. In some embodiments, the detectable moieties having the coumarin core have a peak absorbance wavelength of about 430 nm+/−10 nm and a FWHM of less than about 100 nm. In some embodiments, the detectable moieties having the coumarin core have a peak absorbance wavelength of about 425 nm+/−10 nm and a FWHM of less than about 100 nm. In some embodiments, the detectable moieties having the coumarin core have a peak absorbance wavelength of about 420 nm+/−10 nm and a FWHM of less than about 100 nm. In some embodiments, the detectable moieties having the coumarin core have a peak absorbance wavelength of about 415 nm+/−10 nm and a FWHM of less than about 100 nm. In some embodiments, the detectable moieties having the coumarin core have a peak absorbance wavelength of about 410 nm and a FWHM of less than about 100 nm. In some embodiments, the detectable moieties having the coumarin core have a peak absorbance wavelength of about 405 nm+/−10 nm and a FWHM of less than about 100 nm. In some embodiments, the detectable moieties having the coumarin core have a peak absorbance wavelength of about 400 nm+/−10 nm and a FWHM of less than about 100 nm. In some embodiments, the detectable moieties having the coumarin core have a peak absorbance wavelength of about 395 nm+/−10 nm and a FWHM of less than about 100 nm. In some embodiments, the detectable moieties having the coumarin core have a peak absorbance wavelength of about 390 nm+/−10 nm and a FWHM of less than about 100 nm. In some embodiments, the detectable moieties having the coumarin core have a peak absorbance wavelength of about 385 nm+/−10 nm and a FWHM of less than about 100 nm. In some embodiments, the detectable moieties having the coumarin core have a peak absorbance wavelength of about 380 nm+/−10 nm and a FWHM of less than about 100 nm. In some embodiments, the detectable moieties having the coumarin core have a peak absorbance wavelength of about 375 nm+/−10 nm and a FWHM of less than about 100 nm. In some embodiments, the detectable moieties having the coumarin core have a peak absorbance wavelength of about 370 nm+/−10 nm and a FWHM of less than about 100 nm. In some embodiments, the detectable moieties having the coumarin core have a peak absorbance wavelength of about 365 nm+/−10 nm and a FWHM of less than about 100 nm. In some embodiments, the detectable moieties having the coumarin core have a peak absorbance wavelength of about 360 nm+/−10 nm and a FWHM of less than about 100 nm. In some embodiments, the detectable moieties having the coumarin core have a peak absorbance wavelength of about 355 nm+/−10 nm and a FWHM of less than about 100 nm. In some embodiments, the detectable moieties having the coumarin core have a peak absorbance wavelength of about 350 nm+/−10 nm and a FWHM of less than about 100 nm. In some embodiments, the detectable moieties having the coumarin core have a peak absorbance wavelength of about 345 nm+/−10 nm and a FWHM of less than about 100 nm. In some embodiments, the detectable moieties having the coumarin core have a peak absorbance wavelength of about 340 nm+/−10 nm and a FWHM of less than about 100 nm. In some embodiments, the detectable moieties having the coumarin core have a peak absorbance wavelength of about 335 nm+/−10 nm and a FWHM of less than about 100 nm. In some embodiments, the detectable moieties having the coumarin core have a peak absorbance wavelength of about 330 nm+/−10 nm and a FWHM of less than about 100 nm.

Examples of detectable moieties having a coumarin core include:

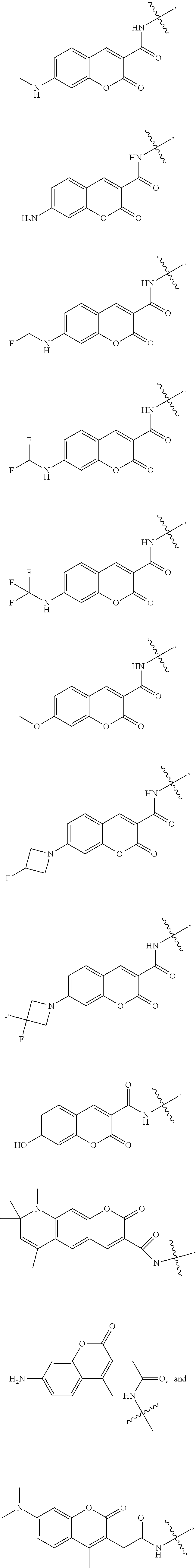

where the symbol “ [symbol] ” refers to the site in which the detectable moiety (here, the coumarin core) is coupled (directly or indirectly) to another moiety of the detectable conjugate (e.g. to a tyramide moiety, to a quinone methide moiety, to a functional group capable or participating in a “click chemistry” reaction, to an antibody, to an enzyme, to a hapten, etc.).

Other suitable detectable moieties having a coumarin core are described in U.S. Pat. No. 10,041,950, the disclosure of which is incorporated by reference herein in its entirety, provided those coumarin-based compounds have a FWHM of less than about 20 nm.

Detectable Moieties within the Infrared Spectrum

In some embodiments, the detectable moieties have a wavelength within the infrared spectrum. In some embodiments, the detectable moieties have a wavelength of greater than about 740 nm. In some embodiments, the detectable moieties have a wavelength of greater than about 750 nm. In some embodiments, the detectable moieties have a wavelength of greater than about 760 nm. In some embodiments, the detectable moieties have a wavelength of greater than about 765 nm. In some embodiments, the detectable moieties have a wavelength of greater than about 770 nm. In some embodiments, the detectable moieties have a wavelength of greater than about 775 nm. In some embodiments, the detectable moieties have a wavelength of greater than about 780 nm. In some embodiments, the detectable moieties have a wavelength of greater than about 785 nm. In some embodiments, the detectable moieties have a wavelength of greater than about 790 nm. In some embodiments the detectable moieties have a wavelength ranging from between about 760 nm to about 1 mm, from about 770 nm to about 1 mm, or from about 780 nm to about 1 mm.

In some embodiments, the detectable moieties have a wavelength of greater than about 740 nm and a FWHM of less than about 200 nm. In some embodiments, the detectable moieties have a wavelength of greater than about 750 nm and a FWHM of less than about 200 nm. In some embodiments, the detectable moieties have a wavelength of greater than about 760 nm and a FWHM of less than about 200 nm. In some embodiments, the detectable moieties have a wavelength of greater than about 765 nm and a FWHM of less than about 200 nm. In some embodiments, the detectable moieties have a wavelength of greater than about 770 nm and a FWHM of less than about 200 nm. In some embodiments, the detectable moieties have a wavelength of greater than about 775 nm and a FWHM of less than about 200 nm. In some embodiments, the detectable moieties have a wavelength of greater than about 780 nm and a FWHM of less than about 200 nm. In some embodiments, the detectable moieties have a wavelength of greater than about 785 nm and a FWHM of less than about 200 nm. In some embodiments, the detectable moieties have a wavelength of greater than about 790 nm and a FWHM of less than about 200 nm.

In some embodiments, the detectable moieties have a wavelength of greater than about 740 nm and a FWHM of less than about 150 nm. In some embodiments, the detectable moieties have a wavelength of greater than about 750 nm and a FWHM of less than about 150 nm. In some embodiments, the detectable moieties have a wavelength of greater than about 760 nm and a FWHM of less than about 150 nm. In some embodiments, the detectable moieties have a wavelength of greater than about 765 nm and a FWHM of less than about 150 nm. In some embodiments, the detectable moieties have a wavelength of greater than about 770 nm and a FWHM of less than about 150 nm. In some embodiments, the detectable moieties have a wavelength of greater than about 775 nm and a FWHM of less than about 150 nm. In some embodiments, the detectable moieties have a wavelength of greater than about 780 nm and a FWHM of less than about 150 nm. In some embodiments, the detectable moieties have a wavelength of greater than about 785 nm and a FWHM of less than about 150 nm. In some embodiments, the detectable moieties have a wavelength of greater than about 790 nm and a FWHM of less than about 150 nm.

In some embodiments, the detectable moieties have a wavelength of greater than about 740 nm and a FWHM of less than about 100 nm. In some embodiments, the detectable moieties have a wavelength of greater than about 750 nm and a FWHM of less than about 100 nm. In some embodiments, the detectable moieties have a wavelength of greater than about 760 nm and a FWHM of less than about 100 nm. In some embodiments, the detectable moieties have a wavelength of greater than about 765 nm and a FWHM of less than about 100 nm. In some embodiments, the detectable moieties have a wavelength of greater than about 770 nm and a FWHM of less than about 100 nm. In some embodiments, the detectable moieties have a wavelength of greater than about 775 nm and a FWHM of less than about 100 nm. In some embodiments, the detectable moieties have a wavelength of greater than about 780 nm and a FWHM of less than about 100 nm. In some embodiments, the detectable moieties have a wavelength of greater than about 785 nm and a FWHM of less than about 100 nm. In some embodiments, the detectable moieties have a wavelength of greater than about 790 nm and a FWHM of less than about 100 nm.

In some embodiments, the detectable moieties have a wavelength of greater than about 740 nm and a FWHM of less than about 70 nm. In some embodiments, the detectable moieties have a wavelength of greater than about 750 nm and a FWHM of less than about 70 nm. In some embodiments, the detectable moieties have a wavelength of greater than about 760 nm and a FWHM of less than about 70 nm. In some embodiments, the detectable moieties have a wavelength of greater than about 765 nm and a FWHM of less than about 70 nm. In some embodiments, the detectable moieties have a wavelength of greater than about 770 nm and a FWHM of less than about 70 nm. In some embodiments, the detectable moieties have a wavelength of greater than about 775 nm and a FWHM of less than about 70 nm. In some embodiments, the detectable moieties have a wavelength of greater than about 780 nm and a FWHM of less than about 70 nm. In some embodiments, the detectable moieties have a wavelength of greater than about 785 nm and a FWHM of less than about 70 nm. In some embodiments, the detectable moieties have a wavelength of greater than about 790 nm and a FWHM of less than about 70 nm.

In some embodiments, the detectable moiety includes or is derived from a heptamethine cyanine core (i.e. the detectable moiety includes a heptamethine cyanine core).

In some embodiments, the heptamethine cyanine core (includes (or is modified to include) one or more electron withdrawing groups (where each electron withdrawing group may be the same or different). In some embodiments, the heptamethine cyanine core includes (or is modified to include) one electron withdrawing group. In some embodiments, the heptamethine cyanine core includes (or is modified to include) two electron withdrawing groups. In some embodiments, the heptamethine cyanine core includes (or is modifying to include) three electron withdrawing groups. In some embodiments, the heptamethine cyanine core includes (or is modifying to include) three different electron withdrawing groups. In some embodiments, the heptamethine cyanine core includes (or is modified to include) four electron withdrawing groups.

In some embodiments, the heptamethine cyanine core (includes (or is modified to include) one or more electron donating groups (where each electron withdrawing group may be the same or different). In some embodiments, the heptamethine cyanine core includes (or is modified to include) one electron donating group. In some embodiments, the heptamethine cyanine core includes (or is modified to include) two electron donating groups. In some embodiments, the heptamethine cyanine core includes (or is modifying to include) three electron donating groups. In some embodiments, the heptamethine cyanine core includes (or is modifying to include) three different electron donating groups. In some embodiments, the heptamethine cyanine core includes (or is modified to include) four electron donating groups.

In some embodiments, the detectable moieties having the heptamethine cyanine core have a wavelength ranging from about 780 nm to about 950 nm. In some embodiments, the detectable moieties having the heptamethine cyanine core have a wavelength ranging from about 810 nm to about 920 nm. In some embodiments, the detectable moieties having the heptamethine cyanine have a wavelength ranging from about 840 nm to about 880 nm.

In some embodiments, the detectable moieties having the heptamethine cyanine core have a wavelength ranging from about 780 nm to about 950 nm and a FWHM of less than about 200 nm. In some embodiments, the detectable moieties having the heptamethine cyanine core have a wavelength ranging from about 810 nm to about 920 nm and a FWHM of less than 200 nm. In some embodiments, the detectable moieties having the heptamethine cyanine have a wavelength ranging from about 840 nm to about 880 nm and a FWHM of less than about 200 nm.

In some embodiments, the detectable moieties having the heptamethine cyanine core have a wavelength ranging from about 780 nm to about 950 nm and a FWHM of less than about 150 nm. In some embodiments, the detectable moieties having the heptamethine cyanine core have a wavelength ranging from about 810 nm to about 920 nm and a FWHM of less than 150 nm. In some embodiments, the detectable moieties having the heptamethine cyanine have a wavelength ranging from about 840 nm to about 880 nm and a FWHM of less than about 150 nm.

In some embodiments, the detectable moieties having the heptamethine cyanine core have a wavelength ranging from about 780 nm to about 950 nm and a FWHM of less than about 100 nm. In some embodiments, the detectable moieties having the heptamethine cyanine core have a wavelength ranging from about 810 nm to about 920 nm and a FWHM of less than 100 nm. In some embodiments, the detectable moieties having the heptamethine cyanine have a wavelength ranging from about 840 nm to about 880 nm and a FWHM of less than about 100 nm.

In some embodiments, the detectable moieties having the heptamethine cyanine core have a peak absorbance wavelength of about 950+/−10 nm. In some embodiments, the detectable moieties having the heptamethine cyanine core have a peak absorbance wavelength of about 945+/−10 nm. In some embodiments, the detectable moieties having the heptamethine cyanine core have a peak absorbance wavelength of about 940+/−10 nm. In some embodiments, the detectable moieties having the heptamethine cyanine core have a peak absorbance wavelength of about 935+/−10 nm. In some embodiments, the detectable moieties having the heptamethine cyanine core have a peak absorbance wavelength of about 930+/−10 nm. In some embodiments, the detectable moieties having the heptamethine cyanine core have a peak absorbance wavelength of about 925+/−10 nm. In some embodiments, the detectable moieties having the heptamethine cyanine core have a peak absorbance wavelength of about 920+/−10 nm. In some embodiments, the detectable moieties having the heptamethine cyanine core have a peak absorbance wavelength of about 915+/−10 nm. In some embodiments, the detectable moieties having the heptamethine cyanine core have a peak absorbance wavelength of about 910+/−10 nm. In some embodiments, the detectable moieties having the heptamethine cyanine core have a peak absorbance wavelength of about 905+/−10 nm. In some embodiments, the detectable moieties having the heptamethine cyanine core have a peak absorbance wavelength of about 900+/−10 nm. In some embodiments, the detectable moieties having the heptamethine cyanine core have a peak absorbance wavelength of about 895+/−10 nm. In some embodiments, the detectable moieties having the heptamethine cyanine core have a peak absorbance wavelength of about 890+/−10 nm. In some embodiments, the detectable moieties having the heptamethine cyanine core have a peak absorbance wavelength of about 885+/−10 nm. In some embodiments, the detectable moieties having the heptamethine cyanine core have a peak absorbance wavelength of about 880+/−10 nm. In some embodiments, the detectable moieties having the heptamethine cyanine core have a peak absorbance wavelength of about 870+/−10 nm. In some embodiments, the detectable moieties having the heptamethine cyanine core have a peak absorbance wavelength of about 865+/−10 nm. In some embodiments, the detectable moieties having the heptamethine cyanine core have a peak absorbance wavelength of about 860+/−10 nm. In some embodiments, the detectable moieties having the heptamethine cyanine core have a peak absorbance wavelength of about 855+/−10 nm. In some embodiments, the detectable moieties having the heptamethine cyanine core have a peak absorbance wavelength of about 850+/−10 nm. In some embodiments, the detectable moieties having the heptamethine cyanine core have a peak absorbance wavelength of about 845+/−10 nm. In some embodiments, the detectable moieties having the heptamethine cyanine core have a peak absorbance wavelength of about 840+/−10 nm. In some embodiments, the detectable moieties having the heptamethine cyanine core have a peak absorbance wavelength of about 835+/−10 nm. In some embodiments, the detectable moieties having the heptamethine cyanine core have a peak absorbance wavelength of about 830+/−10 nm. In some embodiments, the detectable moieties having the heptamethine cyanine core have a peak absorbance wavelength of about 825+/−10 nm. In some embodiments, the detectable moieties having the heptamethine cyanine core have a peak absorbance wavelength of about 820+/−10 nm. In some embodiments, the detectable moieties having the heptamethine cyanine core have a peak absorbance wavelength of about 815+/−10 nm. In some embodiments, the detectable moieties having the heptamethine cyanine core have a peak absorbance wavelength of about 800+/−10 nm. In some embodiments, the detectable moieties having the heptamethine cyanine core have a peak absorbance wavelength of about 795+/−10 nm. In some embodiments, the detectable moieties having the heptamethine cyanine core have a peak absorbance wavelength of about 790+/−10 nm. In some embodiments, the detectable moieties having the heptamethine cyanine core have a peak absorbance wavelength of about 785+/−10 nm. In some embodiments, the detectable moieties having the heptamethine cyanine core have a peak absorbance wavelength of about 780+/−10 nm.

In some embodiments, the detectable moieties having the heptamethine cyanine core have a peak absorbance wavelength of about 950+/−10 nm and a FWHM of less than about 200 nm. In some embodiments, the detectable moieties having the heptamethine cyanine core have a peak absorbance wavelength of about 945+/−10 nm and a FWHM of less than about 200 nm. In some embodiments, the detectable moieties having the heptamethine cyanine core have a peak absorbance wavelength of about 940+/−10 nm and a FWHM of less than about 200 nm. In some embodiments, the detectable moieties having the heptamethine cyanine core have a peak absorbance wavelength of about 935+/−10 nm and a FWHM of less than about 200 nm. In some embodiments, the detectable moieties having the heptamethine cyanine core have a peak absorbance wavelength of about 930+/−10 nm and a FWHM of less than about 200 nm. In some embodiments, the detectable moieties having the heptamethine cyanine core have a peak absorbance wavelength of about 925+/−10 nm and a FWHM of less than about 200 nm. In some embodiments, the detectable moieties having the heptamethine cyanine core have a peak absorbance wavelength of about 920+/−10 nm and a FWHM of less than about 200 nm. In some embodiments, the detectable moieties having the heptamethine cyanine core have a peak absorbance wavelength of about 915+/−10 nm and a FWHM of less than about 200 nm. In some embodiments, the detectable moieties having the heptamethine cyanine core have a peak absorbance wavelength of about 910+/−10 nm and a FWHM of less than about 200 nm. In some embodiments, the detectable moieties having the heptamethine cyanine core have a peak absorbance wavelength of about 905+/−10 nm and a FWHM of less than about 200 nm. In some embodiments, the detectable moieties having the heptamethine cyanine core have a peak absorbance wavelength of about 900+/−10 nm and a FWHM of less than about 200 nm. In some embodiments, the detectable moieties having the heptamethine cyanine core have a peak absorbance wavelength of about 895+/−10 nm and a FWHM of less than about 200 nm. In some embodiments, the detectable moieties having the heptamethine cyanine core have a peak absorbance wavelength of about 890+/−10 nm and a FWHM of less than about 200 nm. In some embodiments, the detectable moieties having the heptamethine cyanine core have a peak absorbance wavelength of about 885+/−10 nm and a FWHM of less than about 200 nm. In some embodiments, the detectable moieties having the heptamethine cyanine core have a peak absorbance wavelength of about 880+/−10 nm and a FWHM of less than about 200 nm. In some embodiments, the detectable moieties having the heptamethine cyanine core have a peak absorbance wavelength of about 870+/−10 nm and a FWHM of less than about 200 nm. In some embodiments, the detectable moieties having the heptamethine cyanine core have a peak absorbance wavelength of about 865+/−10 nm and a FWHM of less than about 200 nm. In some embodiments, the detectable moieties having the heptamethine cyanine core have a peak absorbance wavelength of about 860+/−10 nm and a FWHM of less than about 200 nm. In some embodiments, the detectable moieties having the heptamethine cyanine core have a peak absorbance wavelength of about 855+/−10 nm and a FWHM of less than about 200 nm. In some embodiments, the detectable moieties having the heptamethine cyanine core have a peak absorbance wavelength of about 850+/−10 nm and a FWHM of less than about 200 nm. In some embodiments, the detectable moieties having the heptamethine cyanine core have a peak absorbance wavelength of about 845+/−10 nm and a FWHM of less than about 200 nm. In some embodiments, the detectable moieties having the heptamethine cyanine core have a peak absorbance wavelength of about 840+/−10 nm and a FWHM of less than about 200 nm. In some embodiments, the detectable moieties having the heptamethine cyanine core have a peak absorbance wavelength of about 835+/−10 nm and a FWHM of less than about 200 nm. In some embodiments, the detectable moieties having the heptamethine cyanine core have a peak absorbance wavelength of about 830+/−10 nm and a FWHM of less than about 200 nm. In some embodiments, the detectable moieties having the heptamethine cyanine core have a peak absorbance wavelength of about 825+/−10 nm and a FWHM of less than about 200 nm. In some embodiments, the detectable moieties having the heptamethine cyanine core have a peak absorbance wavelength of about 820+/−10 nm and a FWHM of less than about 200 nm. In some embodiments, the detectable moieties having the heptamethine cyanine core have a peak absorbance wavelength of about 815+/−10 nm and a FWHM of less than about 200 nm. In some embodiments, the detectable moieties having the heptamethine cyanine core have a peak absorbance wavelength of about 800+/−10 nm and a FWHM of less than about 200 nm. In some embodiments, the detectable moieties having the heptamethine cyanine core have a peak absorbance wavelength of about 795+/−10 nm and a FWHM of less than about 200 nm. In some embodiments, the detectable moieties having the heptamethine cyanine core have a peak absorbance wavelength of about 790+/−10 nm and a FWHM of less than about 200 nm. In some embodiments, the detectable moieties having the heptamethine cyanine core have a peak absorbance wavelength of about 785+/−10 nm and a FWHM of less than about 200 nm. In some embodiments, the detectable moieties having the heptamethine cyanine core have a peak absorbance wavelength of about 780+/−10 nm and a FWHM of less than about 200 nm.

In some embodiments, the detectable moieties having the heptamethine cyanine core have a peak absorbance wavelength of about 950+/−150 nm and a FWHM of less than about 150 nm. In some embodiments, the detectable moieties having the heptamethine cyanine core have a peak absorbance wavelength of about 945+/−10 nm and a FWHM of less than about 150 nm. In some embodiments, the detectable moieties having the heptamethine cyanine core have a peak absorbance wavelength of about 940+/−10 nm and a FWHM of less than about 150 nm. In some embodiments, the detectable moieties having the heptamethine cyanine core have a peak absorbance wavelength of about 935+/−10 nm and a FWHM of less than about 150 nm. In some embodiments, the detectable moieties having the heptamethine cyanine core have a peak absorbance wavelength of about 930+/−10 nm and a FWHM of less than about 150 nm. In some embodiments, the detectable moieties having the heptamethine cyanine core have a peak absorbance wavelength of about 925+/−10 nm and a FWHM of less than about 150 nm. In some embodiments, the detectable moieties having the heptamethine cyanine core have a peak absorbance wavelength of about 920+/−10 nm and a FWHM of less than about 150 nm. In some embodiments, the detectable moieties having the heptamethine cyanine core have a peak absorbance wavelength of about 915+/−10 nm and a FWHM of less than about 150 nm. In some embodiments, the detectable moieties having the heptamethine cyanine core have a peak absorbance wavelength of about 910+/−10 nm and a FWHM of less than about 150 nm. In some embodiments, the detectable moieties having the heptamethine cyanine core have a peak absorbance wavelength of about 905+/−10 nm and a FWHM of less than about 150 nm. In some embodiments, the detectable moieties having the heptamethine cyanine core have a peak absorbance wavelength of about 900+/−10 nm and a FWHM of less than about 150 nm. In some embodiments, the detectable moieties having the heptamethine cyanine core have a peak absorbance wavelength of about 895+/−10 nm and a FWHM of less than about 150 nm. In some embodiments, the detectable moieties having the heptamethine cyanine core have a peak absorbance wavelength of about 890+/−10 nm and a FWHM of less than about 150 nm. In some embodiments, the detectable moieties having the heptamethine cyanine core have a peak absorbance wavelength of about 885+/−10 nm and a FWHM of less than about 150 nm. In some embodiments, the detectable moieties having the heptamethine cyanine core have a peak absorbance wavelength of about 880+/−10 nm and a FWHM of less than about 150 nm. In some embodiments, the detectable moieties having the heptamethine cyanine core have a peak absorbance wavelength of about 870+/−10 nm and a FWHM of less than about 150 nm. In some embodiments, the detectable moieties having the heptamethine cyanine core have a peak absorbance wavelength of about 865+/−10 nm and a FWHM of less than about 150 nm. In some embodiments, the detectable moieties having the heptamethine cyanine core have a peak absorbance wavelength of about 860+/−10 nm and a FWHM of less than about 150 nm. In some embodiments, the detectable moieties having the heptamethine cyanine core have a peak absorbance wavelength of about 855+/−10 nm and a FWHM of less than about 150 nm. In some embodiments, the detectable moieties having the heptamethine cyanine core have a peak absorbance wavelength of about 850+/−10 nm and a FWHM of less than about 150 nm. In some embodiments, the detectable moieties having the heptamethine cyanine core have a peak absorbance wavelength of about 845+/−10 nm and a FWHM of less than about 150 nm. In some embodiments, the detectable moieties having the heptamethine cyanine core have a peak absorbance wavelength of about 840+/−10 nm and a FWHM of less than about 150 nm. In some embodiments, the detectable moieties having the heptamethine cyanine core have a peak absorbance wavelength of about 835+/−10 nm and a FWHM of less than about 150 nm. In some embodiments, the detectable moieties having the heptamethine cyanine core have a peak absorbance wavelength of about 830+/−10 nm and a FWHM of less than about 150 nm. In some embodiments, the detectable moieties having the heptamethine cyanine core have a peak absorbance wavelength of about 825+/−10 nm and a FWHM of less than about 150 nm. In some embodiments, the detectable moieties having the heptamethine cyanine core have a peak absorbance wavelength of about 820+/−10 nm and a FWHM of less than about 150 nm. In some embodiments, the detectable moieties having the heptamethine cyanine core have a peak absorbance wavelength of about 815+/−10 nm and a FWHM of less than about 150 nm. In some embodiments, the detectable moieties having the heptamethine cyanine core have a peak absorbance wavelength of about 800+/−10 nm and a FWHM of less than about 150 nm. In some embodiments, the detectable moieties having the heptamethine cyanine core have a peak absorbance wavelength of about 795+/−10 nm and a FWHM of less than about 150 nm. In some embodiments, the detectable moieties having the heptamethine cyanine core have a peak absorbance wavelength of about 790+/−10 nm and a FWHM of less than about 150 nm. In some embodiments, the detectable moieties having the heptamethine cyanine core have a peak absorbance wavelength of about 785+/−10 nm and a FWHM of less than about 150 nm. In some embodiments, the detectable moieties having the heptamethine cyanine core have a peak absorbance wavelength of about 780+/−10 nm and a FWHM of less than about 150 nm.

In some embodiments, the detectable moieties having the heptamethine cyanine core have a peak absorbance wavelength of about 950+/−10 nm and a FWHM of less than about 100 nm. In some embodiments, the detectable moieties having the heptamethine cyanine core have a peak absorbance wavelength of about 945+/−10 nm and a FWHM of less than about 100 nm. In some embodiments, the detectable moieties having the heptamethine cyanine core have a peak absorbance wavelength of about 940+/−10 nm and a FWHM of less than about 100 nm. In some embodiments, the detectable moieties having the heptamethine cyanine core have a peak absorbance wavelength of about 935+/−10 nm and a FWHM of less than about 100 nm. In some embodiments, the detectable moieties having the heptamethine cyanine core have a peak absorbance wavelength of about 930+/−10 nm and a FWHM of less than about 100 nm. In some embodiments, the detectable moieties having the heptamethine cyanine core have a peak absorbance wavelength of about 925+/−10 nm and a FWHM of less than about 100 nm. In some embodiments, the detectable moieties having the heptamethine cyanine core have a peak absorbance wavelength of about 920+/−10 nm and a FWHM of less than about 100 nm. In some embodiments, the detectable moieties having the heptamethine cyanine core have a peak absorbance wavelength of about 915+/−10 nm and a FWHM of less than about 100 nm. In some embodiments, the detectable moieties having the heptamethine cyanine core have a peak absorbance wavelength of about 910+/−10 nm and a FWHM of less than about 100 nm. In some embodiments, the detectable moieties having the heptamethine cyanine core have a peak absorbance wavelength of about 905+/−10 nm and a FWHM of less than about 100 nm. In some embodiments, the detectable moieties having the heptamethine cyanine core have a peak absorbance wavelength of about 900+/−10 nm and a FWHM of less than about 100 nm. In some embodiments, the detectable moieties having the heptamethine cyanine core have a peak absorbance wavelength of about 895+/−10 nm and a FWHM of less than about 100 nm. In some embodiments, the detectable moieties having the heptamethine cyanine core have a peak absorbance wavelength of about 890+/−10 nm and a FWHM of less than about 100 nm. In some embodiments, the detectable moieties having the heptamethine cyanine core have a peak absorbance wavelength of about 885+/−10 nm and a FWHM of less than about 100 nm. In some embodiments, the detectable moieties having the heptamethine cyanine core have a peak absorbance wavelength of about 880+/−10 nm and a FWHM of less than about 100 nm. In some embodiments, the detectable moieties having the heptamethine cyanine core have a peak absorbance wavelength of about 870+/−10 nm and a FWHM of less than about 100 nm. In some embodiments, the detectable moieties having the heptamethine cyanine core have a peak absorbance wavelength of about 865+/−10 nm and a FWHM of less than about 100 nm. In some embodiments, the detectable moieties having the heptamethine cyanine core have a peak absorbance wavelength of about 860+/−10 nm and a FWHM of less than about 100 nm. In some embodiments, the detectable moieties having the heptamethine cyanine core have a peak absorbance wavelength of about 855+/−10 nm and a FWHM of less than about 100 nm. In some embodiments, the detectable moieties having the heptamethine cyanine core have a peak absorbance wavelength of about 850+/−10 nm and a FWHM of less than about 100 nm. In some embodiments, the detectable moieties having the heptamethine cyanine core have a peak absorbance wavelength of about 845+/−10 nm and a FWHM of less than about 100 nm. In some embodiments, the detectable moieties having the heptamethine cyanine core have a peak absorbance wavelength of about 840+/−10 nm and a FWHM of less than about 100 nm. In some embodiments, the detectable moieties having the heptamethine cyanine core have a peak absorbance wavelength of about 835+/−10 nm and a FWHM of less than about 100 nm. In some embodiments, the detectable moieties having the heptamethine cyanine core have a peak absorbance wavelength of about 830+/−10 nm and a FWHM of less than about 100 nm. In some embodiments, the detectable moieties having the heptamethine cyanine core have a peak absorbance wavelength of about 825+/−10 nm and a FWHM of less than about 100 nm. In some embodiments, the detectable moieties having the heptamethine cyanine core have a peak absorbance wavelength of about 820+/−10 nm and a FWHM of less than about 100 nm. In some embodiments, the detectable moieties having the heptamethine cyanine core have a peak absorbance wavelength of about 815+/−10 nm and a FWHM of less than about 100 nm. In some embodiments, the detectable moieties having the heptamethine cyanine core have a peak absorbance wavelength of about 800+/−10 nm and a FWHM of less than about 100 nm. In some embodiments, the detectable moieties having the heptamethine cyanine core have a peak absorbance wavelength of about 795+/−10 nm and a FWHM of less than about 100 nm. In some embodiments, the detectable moieties having the heptamethine cyanine core have a peak absorbance wavelength of about 790+/−10 nm and a FWHM of less than about 100 nm. In some embodiments, the detectable moieties having the heptamethine cyanine core have a peak absorbance wavelength of about 785+/−10 nm and a FWHM of less than about 100 nm. In some embodiments, the detectable moieties having the heptamethine cyanine core have a peak absorbance wavelength of about 780+/−10 nm and a FWHM of less than about 100 nm.

In some embodiments, the detectable moiety includes or is derived from a croconate core (i.e. the detectable moiety includes a croconate core). In some embodiments, the croconate core (includes (or is modified to include) one or more electron withdrawing groups (where each electron withdrawing group may be the same or different). In some embodiments, the croconate core includes (or is modified to include) one electron withdrawing group. In some embodiments, the croconate core includes (or is modified to include) two electron withdrawing groups. In some embodiments, the croconate core includes (or is modifying to include) three electron withdrawing groups. In some embodiments, the croconate core includes (or is modifying to include) three different electron withdrawing groups. In some embodiments, the croconate core includes (or is modified to include) four electron withdrawing groups.

In some embodiments, the croconate core (includes (or is modified to include) one or more electron donating groups (where each electron withdrawing group may be the same or different). In some embodiments, the croconate core includes (or is modified to include) one electron donating group. In some embodiments, the croconate core includes (or is modified to include) two electron donating groups. In some embodiments, the croconate core includes (or is modifying to include) three electron donating groups. In some embodiments, the croconate core includes (or is modifying to include) three different electron donating groups. In some embodiments, the croconate core includes (or is modified to include) four electron donating groups.

In some embodiments, the detectable moieties having the croconate core have a wavelength ranging from about 780 nm to about 900 nm. In some embodiments, the detectable moieties having the croconate core have a wavelength ranging from about 800 nm to about 880 nm. In some embodiments, the detectable moieties having the croconate core have a wavelength ranging from about 820 nm to about 860 nm.

In some embodiments, the detectable moieties having the croconate core have a wavelength ranging from about 780 nm to about 900 nm and a FWHM of less than about 200 nm. In some embodiments, the detectable moieties having the croconate core have a wavelength ranging from about 800 nm to about 880 nm and a FWHM of less than about 200 nm. In some embodiments, the detectable moieties having the croconate core have a wavelength ranging from about 820 nm to about 860 nm and a FWHM of less than about 200 nm.

In some embodiments, the detectable moieties having the croconate core have a peak absorbance wavelength of about 900+/−10 nm. In some embodiments, the detectable moieties having the croconate core have a peak absorbance wavelength of about 895+/−10 nm. In some embodiments, the detectable moieties having the croconate core have a peak absorbance wavelength of about 890+/−10 nm. In some embodiments, the detectable moieties having the croconate core have a peak absorbance wavelength of about 885+/−10 nm. In some embodiments, the detectable moieties having the croconate core have a peak absorbance wavelength of about 880+/−10 nm. In some embodiments, the detectable moieties having the croconate core have a peak absorbance wavelength of about 870+/−10 nm. In some embodiments, the detectable moieties having the croconate core have a peak absorbance wavelength of about 865+/−10 nm. In some embodiments, the detectable moieties having the croconate core have a peak absorbance wavelength of about 860+/−10 nm. In some embodiments, the detectable moieties having the croconate core have a peak absorbance wavelength of about 855+/−10 nm. In some embodiments, the detectable moieties having the croconate core have a peak absorbance wavelength of about 850+/−10 nm. In some embodiments, the detectable moieties having the croconate core have a peak absorbance wavelength of about 845+/−10 nm. In some embodiments, the detectable moieties having the croconate core have a peak absorbance wavelength of about 840+/−10 nm. In some embodiments, the detectable moieties having the croconate core have a peak absorbance wavelength of about 835+/−10 nm. In some embodiments, the detectable moieties having the croconate core have a peak absorbance wavelength of about 830+/−10 nm. In some embodiments, the detectable moieties having the croconate core have a peak absorbance wavelength of about 825+/−10 nm. In some embodiments, the detectable moieties having the croconate core have a peak absorbance wavelength of about 820+/−10 nm. In some embodiments, the detectable moieties having the croconate core have a peak absorbance wavelength of about 815+/−10 nm. In some embodiments, the detectable moieties having the croconate core have a peak absorbance wavelength of about 800+/−10 nm. In some embodiments, the detectable moieties having the croconate core have a peak absorbance wavelength of about 795+/−10 nm. In some embodiments, the detectable moieties having the croconate core have a peak absorbance wavelength of about 790+/−10 nm. In some embodiments, the detectable moieties having the croconate core have a peak absorbance wavelength of about 785+/−10 nm. In some embodiments, the detectable moieties having the croconate core have a peak absorbance wavelength of about 780+/−10 nm.

In some embodiments, the detectable moieties having the croconate core have a peak absorbance wavelength of about 900+/−10 nm and a FWHM of less than about 200 nm. In some embodiments, the detectable moieties having the croconate core have a peak absorbance wavelength of about 895+/−10 nm and a FWHM of less than about 200 nm. In some embodiments, the detectable moieties having the croconate core have a peak absorbance wavelength of about 890+/−10 nm and a FWHM of less than about 200 nm. In some embodiments, the detectable moieties having the croconate core have a peak absorbance wavelength of about 885+/−10 nm and a FWHM of less than about 200 nm. In some embodiments, the detectable moieties having the croconate core have a peak absorbance wavelength of about 880+/−10 nm and a FWHM of less than about 200 nm. In some embodiments, the detectable moieties having the croconate core have a peak absorbance wavelength of about 870+/−10 nm and a FWHM of less than about 200 nm. In some embodiments, the detectable moieties having the croconate core have a peak absorbance wavelength of about 865+/−10 nm and a FWHM of less than about 200 nm. In some embodiments, the detectable moieties having the croconate core have a peak absorbance wavelength of about 860+/−10 nm and a FWHM of less than about 200 nm. In some embodiments, the detectable moieties having the croconate core have a peak absorbance wavelength of about 855+/−10 nm and a FWHM of less than about 200 nm. In some embodiments, the detectable moieties having the croconate core have a peak absorbance wavelength of about 850+/−10 nm and a FWHM of less than about 200 nm. In some embodiments, the detectable moieties having the croconate core have a peak absorbance wavelength of about 845+/−10 nm and a FWHM of less than about 200 nm. In some embodiments, the detectable moieties having the croconate core have a peak absorbance wavelength of about 840+/−10 nm and a FWHM of less than about 200 nm. In some embodiments, the detectable moieties having the croconate core have a peak absorbance wavelength of about 835+/−10 nm and a FWHM of less than about 200 nm. In some embodiments, the detectable moieties having the croconate core have a peak absorbance wavelength of about 830+/−10 nm and a FWHM of less than about 200 nm. In some embodiments, the detectable moieties having the croconate core have a peak absorbance wavelength of about 825+/−10 nm and a FWHM of less than about 200 nm. In some embodiments, the detectable moieties having the croconate core have a peak absorbance wavelength of about 820+/−10 nm and a FWHM of less than about 200 nm. In some embodiments, the detectable moieties having the croconate core have a peak absorbance wavelength of about 815+/−10 nm and a FWHM of less than about 200 nm. In some embodiments, the detectable moieties having the croconate core have a peak absorbance wavelength of about 800+/−10 nm and a FWHM of less than about 200 nm. In some embodiments, the detectable moieties having the croconate core have a peak absorbance wavelength of about 795+/−10 nm and a FWHM of less than about 200 nm. In some embodiments, the detectable moieties having the croconate core have a peak absorbance wavelength of about 790+/−10 nm and a FWHM of less than about 200 nm. In some embodiments, the detectable moieties having the croconate core have a peak absorbance wavelength of about 785+/−10 nm and a FWHM of less than about 200 nm. In some embodiments, the detectable moieties having the croconate core have a peak absorbance wavelength of about 780+/−10 nm and a FWHM of less than about 200 nm.

In some embodiments, the detectable moieties having the croconate core have a peak absorbance wavelength of about 900+/−10 nm and a FWHM of less than about 150 nm. In some embodiments, the detectable moieties having the croconate core have a peak absorbance wavelength of about 895+/−10 nm and a FWHM of less than about 150 nm. In some embodiments, the detectable moieties having the croconate core have a peak absorbance wavelength of about 890+/−10 nm and a FWHM of less than about 150 nm. In some embodiments, the detectable moieties having the croconate core have a peak absorbance wavelength of about 885+/−10 nm and a FWHM of less than about 150 nm. In some embodiments, the detectable moieties having the croconate core have a peak absorbance wavelength of about 880+/−10 nm and a FWHM of less than about 150 nm. In some embodiments, the detectable moieties having the croconate core have a peak absorbance wavelength of about 870+/−10 nm and a FWHM of less than about 150 nm. In some embodiments, the detectable moieties having the croconate core have a peak absorbance wavelength of about 865+/−10 nm and a FWHM of less than about 150 nm. In some embodiments, the detectable moieties having the croconate core have a peak absorbance wavelength of about 860+/−10 nm and a FWHM of less than about 150 nm. In some embodiments, the detectable moieties having the croconate core have a peak absorbance wavelength of about 855+/−10 nm and a FWHM of less than about 150 nm. In some embodiments, the detectable moieties having the croconate core have a peak absorbance wavelength of about 850+/−10 nm and a FWHM of less than about 150 nm. In some embodiments, the detectable moieties having the croconate core have a peak absorbance wavelength of about 845+/−10 nm and a FWHM of less than about 150 nm. In some embodiments, the detectable moieties having the croconate core have a peak absorbance wavelength of about 840+/−10 nm and a FWHM of less than about 150 nm. In some embodiments, the detectable moieties having the croconate core have a peak absorbance wavelength of about 835+/−10 nm and a FWHM of less than about 150 nm. In some embodiments, the detectable moieties having the croconate core have a peak absorbance wavelength of about 830+/−10 nm and a FWHM of less than about 150 nm. In some embodiments, the detectable moieties having the croconate core have a peak absorbance wavelength of about 825+/−10 nm and a FWHM of less than about 150 nm. In some embodiments, the detectable moieties having the croconate core have a peak absorbance wavelength of about 820+/−10 nm and a FWHM of less than about 150 nm. In some embodiments, the detectable moieties having the croconate core have a peak absorbance wavelength of about 815+/−10 nm and a FWHM of less than about 150 nm. In some embodiments, the detectable moieties having the croconate core have a peak absorbance wavelength of about 800+/−10 nm and a FWHM of less than about 150 nm. In some embodiments, the detectable moieties having the croconate core have a peak absorbance wavelength of about 795+/−10 nm and a FWHM of less than about 150 nm. In some embodiments, the detectable moieties having the croconate core have a peak absorbance wavelength of about 790+/−10 nm and a FWHM of less than about 150 nm. In some embodiments, the detectable moieties having the croconate core have a peak absorbance wavelength of about 785+/−10 nm and a FWHM of less than about 150 nm. In some embodiments, the detectable moieties having the croconate core have a peak absorbance wavelength of about 780+/−10 nm and a FWHM of less than about 150 nm.

In some embodiments, the detectable moieties having the croconate core have a peak absorbance wavelength of about 900+/−10 nm and a FWHM of less than about 100 nm. In some embodiments, the detectable moieties having the croconate core have a peak absorbance wavelength of about 895+/−10 nm and a FWHM of less than about 100 nm. In some embodiments, the detectable moieties having the croconate core have a peak absorbance wavelength of about 890+/−10 nm and a FWHM of less than about 100 nm. In some embodiments, the detectable moieties having the croconate core have a peak absorbance wavelength of about 885+/−10 nm and a FWHM of less than about 100 nm. In some embodiments, the detectable moieties having the croconate core have a peak absorbance wavelength of about 880+/−10 nm and a FWHM of less than about 100 nm. In some embodiments, the detectable moieties having the croconate core have a peak absorbance wavelength of about 870+/−10 nm and a FWHM of less than about 100 nm. In some embodiments, the detectable moieties having the croconate core have a peak absorbance wavelength of about 865+/−10 nm and a FWHM of less than about 100 nm. In some embodiments, the detectable moieties having the croconate core have a peak absorbance wavelength of about 860+/−10 nm and a FWHM of less than about 100 nm. In some embodiments, the detectable moieties having the croconate core have a peak absorbance wavelength of about 855+/−10 nm and a FWHM of less than about 100 nm. In some embodiments, the detectable moieties having the croconate core have a peak absorbance wavelength of about 850+/−10 nm and a FWHM of less than about 100 nm. In some embodiments, the detectable moieties having the croconate core have a peak absorbance wavelength of about 845+/−10 nm and a FWHM of less than about 100 nm. In some embodiments, the detectable moieties having the croconate core have a peak absorbance wavelength of about 840+/−10 nm and a FWHM of less than about 100 nm. In some embodiments, the detectable moieties having the croconate core have a peak absorbance wavelength of about 835+/−10 nm and a FWHM of less than about 100 nm. In some embodiments, the detectable moieties having the croconate core have a peak absorbance wavelength of about 830+/−10 nm and a FWHM of less than about 100 nm. In some embodiments, the detectable moieties having the croconate core have a peak absorbance wavelength of about 825+/−10 nm and a FWHM of less than about 100 nm. In some embodiments, the detectable moieties having the croconate core have a peak absorbance wavelength of about 820+/−10 nm and a FWHM of less than about 100 nm. In some embodiments, the detectable moieties having the croconate core have a peak absorbance wavelength of about 815+/−10 nm and a FWHM of less than about 100 nm. In some embodiments, the detectable moieties having the croconate core have a peak absorbance wavelength of about 800+/−10 nm and a FWHM of less than about 100 nm. In some embodiments, the detectable moieties having the croconate core have a peak absorbance wavelength of about 795+/−10 nm and a FWHM of less than about 100 nm. In some embodiments, the detectable moieties having the croconate core have a peak absorbance wavelength of about 790+/−10 nm and a FWHM of less than about 100 nm. In some embodiments, the detectable moieties having the croconate core have a peak absorbance wavelength of about 785+/−10 nm and a FWHM of less than about 100 nm. In some embodiments, the detectable moieties having the croconate core have a peak absorbance wavelength of about 780+/−10 nm and a FWHM of less than about 100 nm.

Non-limiting examples of detectable moieties including a heptamethine cyanine core or a croconate core include:

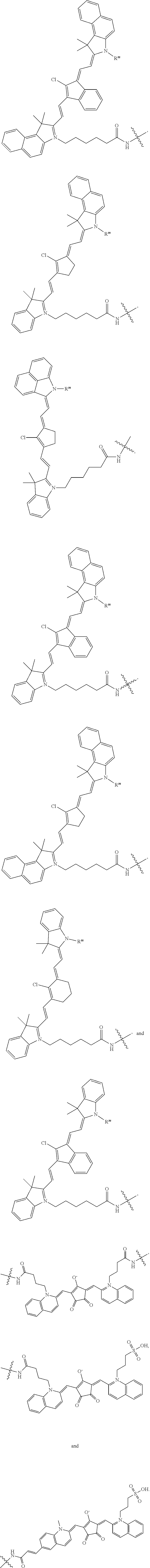

where the symbol “ [symbol] ” refers to the site in which the detectable moiety (here, the heptamethine cyanine core or the croconate core include) is coupled (directly or indirectly) to another moiety of the detectable conjugate (e.g. to a tyramide moiety, to a quinone methide moiety, to a functional group capable or participating in a “click chemistry” reaction, to an antibody, to an enzyme, to a hapten, etc.). Yet other examples are disclosed herein.

Other detectable moieties suitable for use with the presently disclosed methods include any of those having a diazo-core, such as those disclosed in U.S. Pat. No. 10,041,950, the disclosure of which is incorporated by reference herein in its entirety.

Other detectable moieties having a wavelength within the ultraviolet spectrum or the infrared spectrum and which are suitable are disclosed in U.S. Pat. No. 10,041,950, the disclosure of which is incorporated by reference herein in its entirety.

Non-limiting examples of detectable conjugates including (i) a tyramide or a quinone methide moiety, coupled to (ii) a detectable moiety include the following:

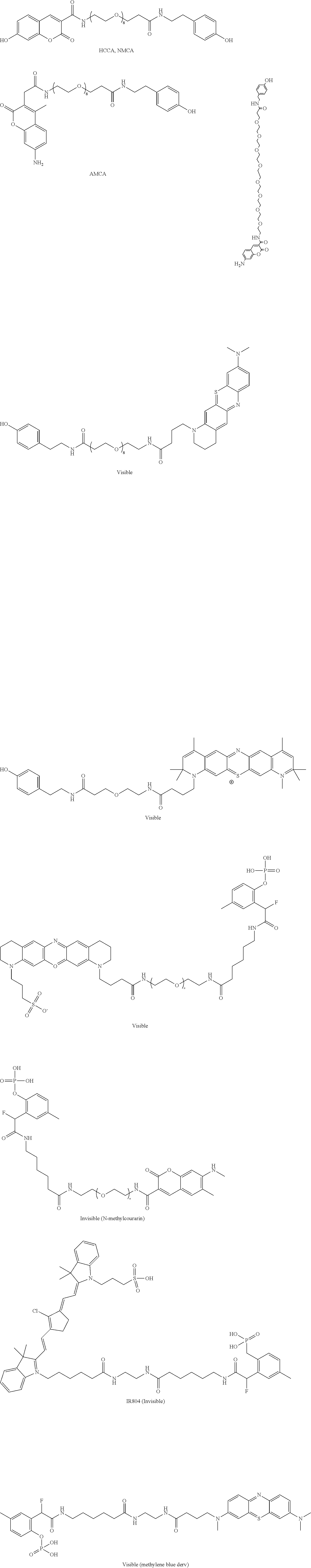

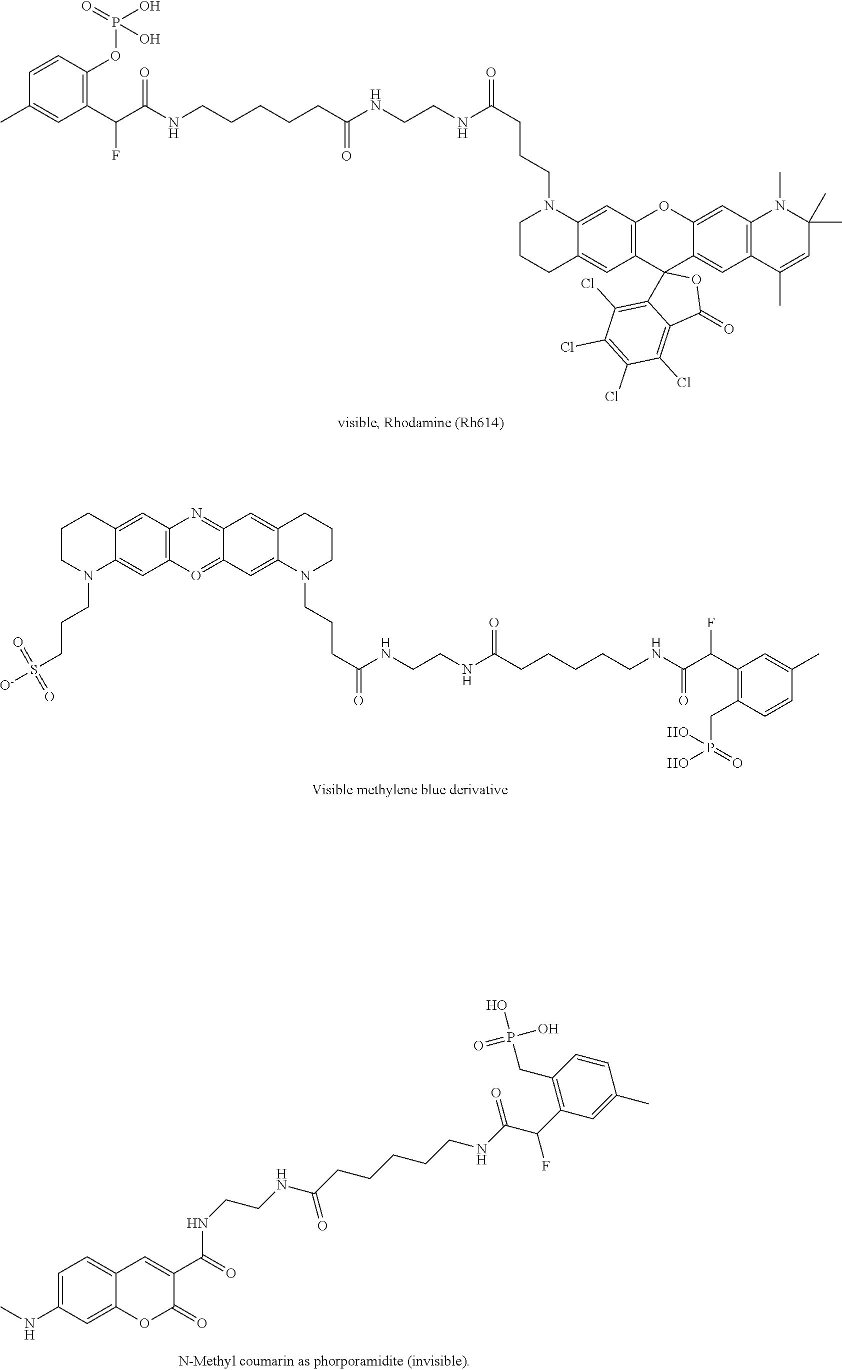

Non-limiting examples of detectable conjugates including (i) a functional group capable of participating in a click chemistry reaction, coupled to (ii) a detectable moiety include the following:

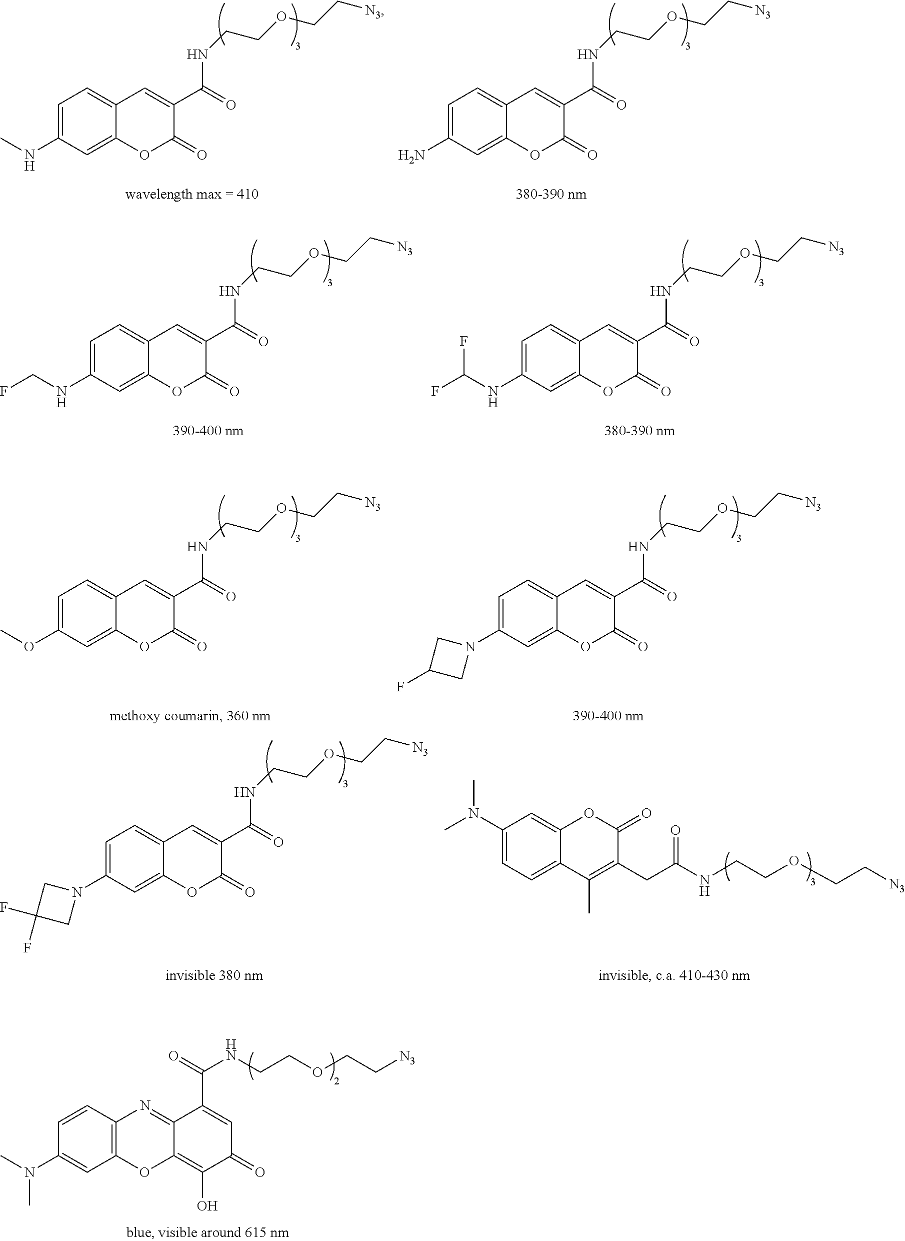

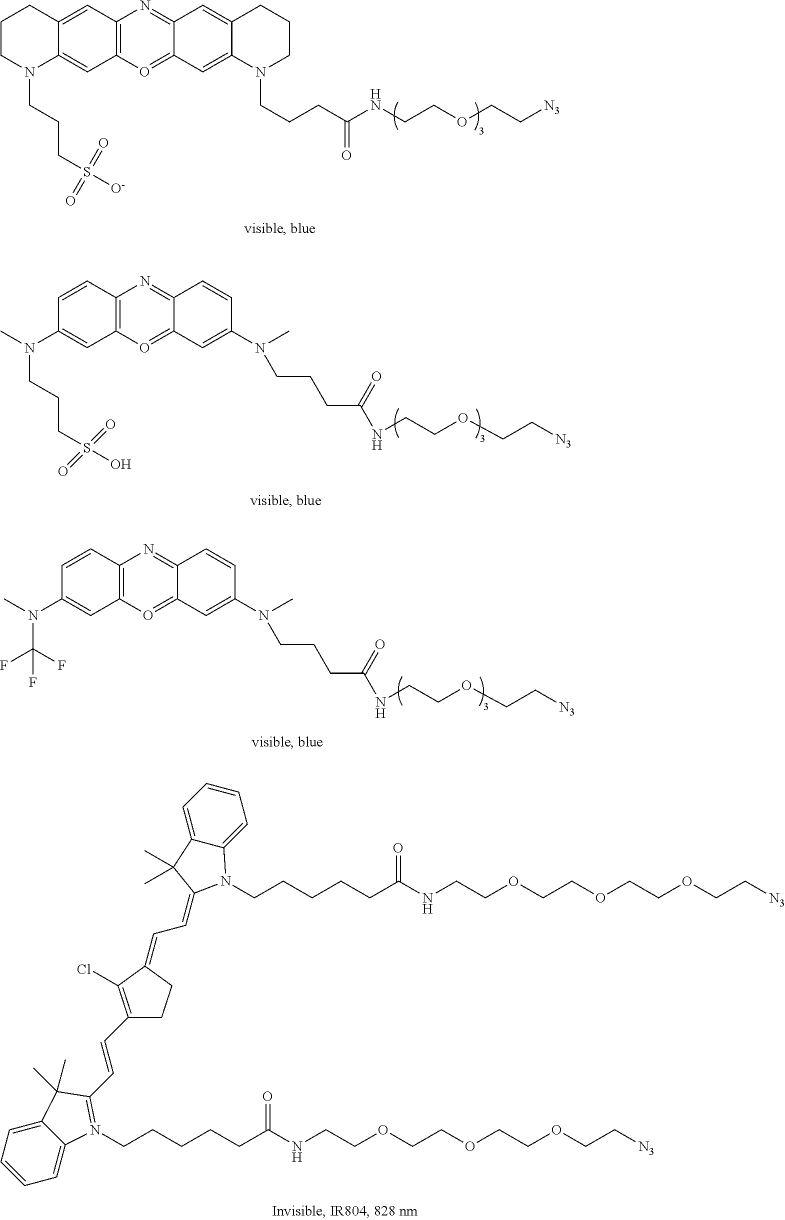

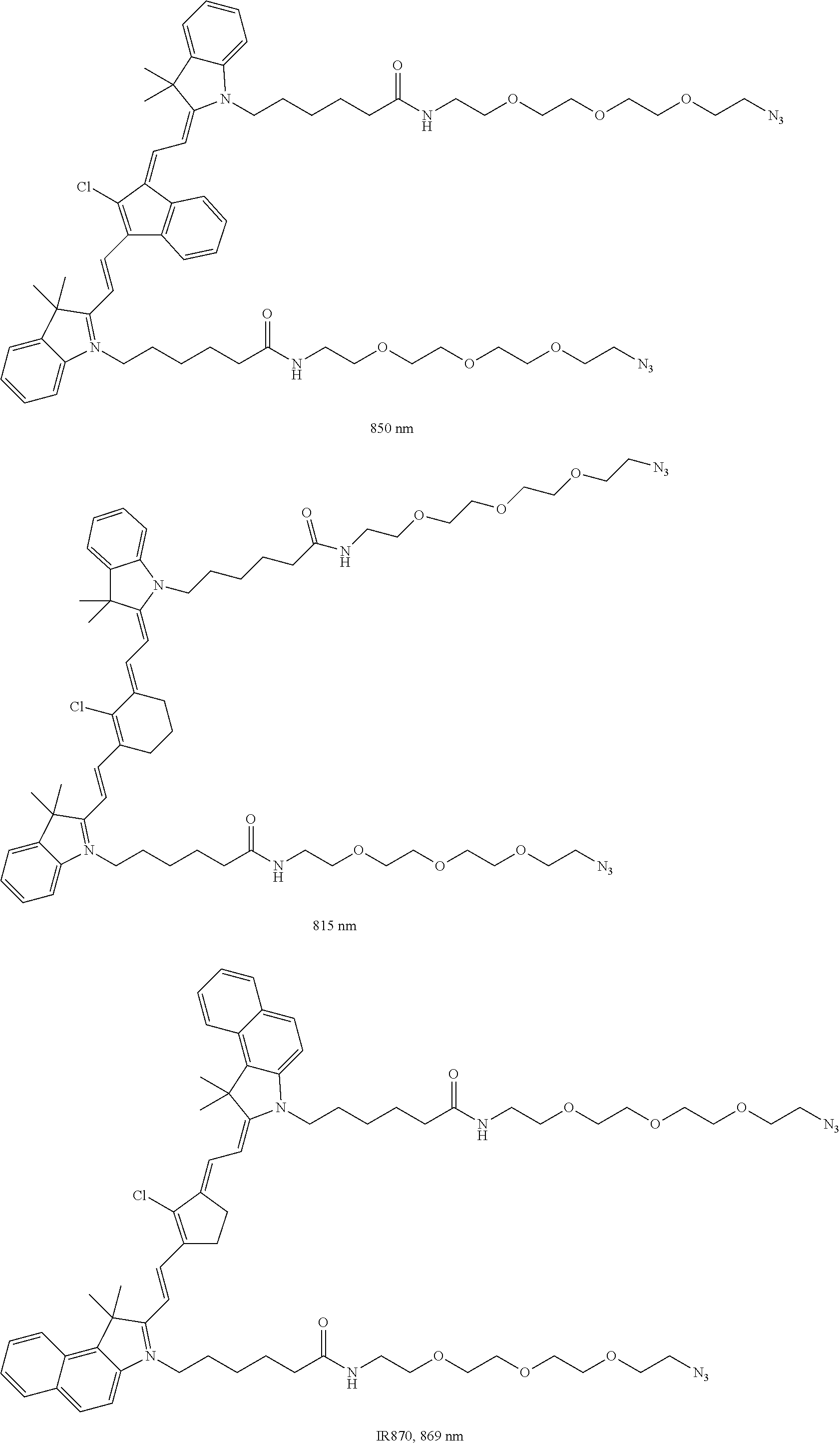

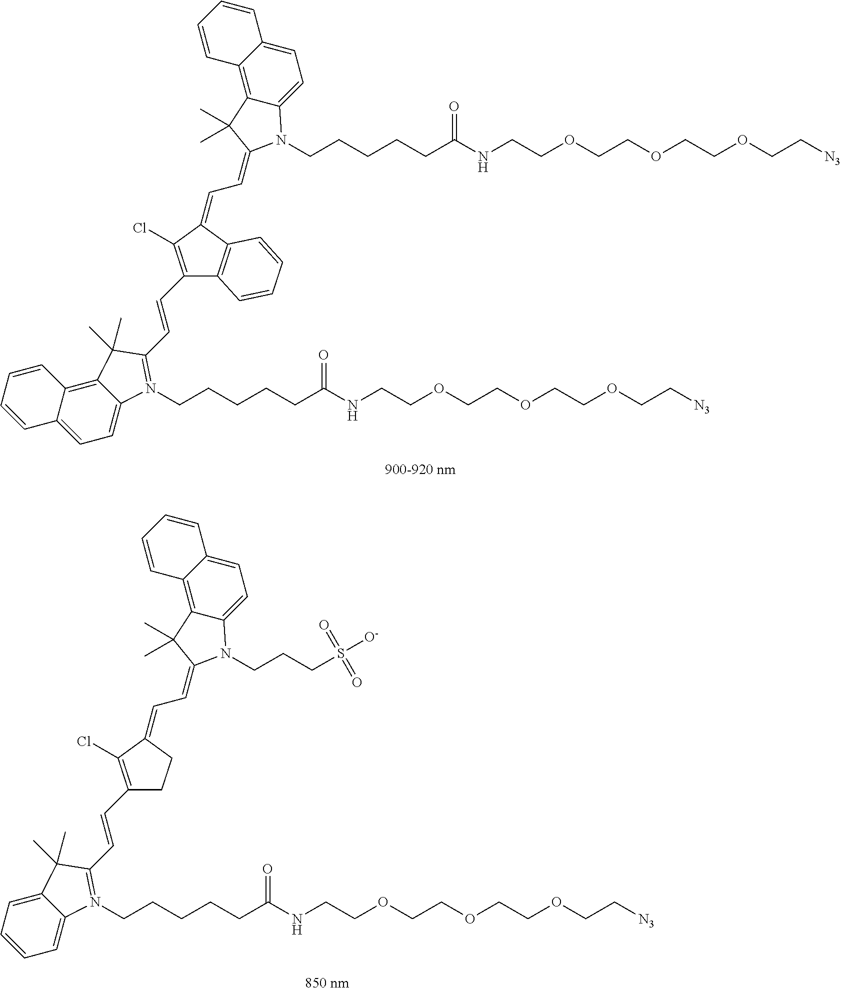

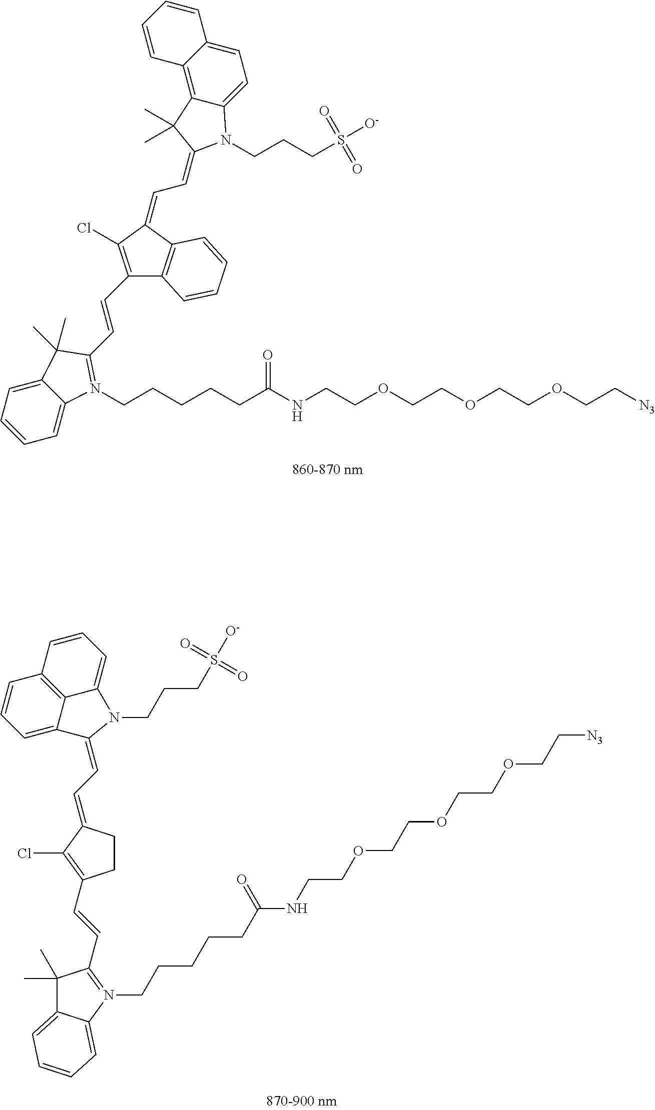

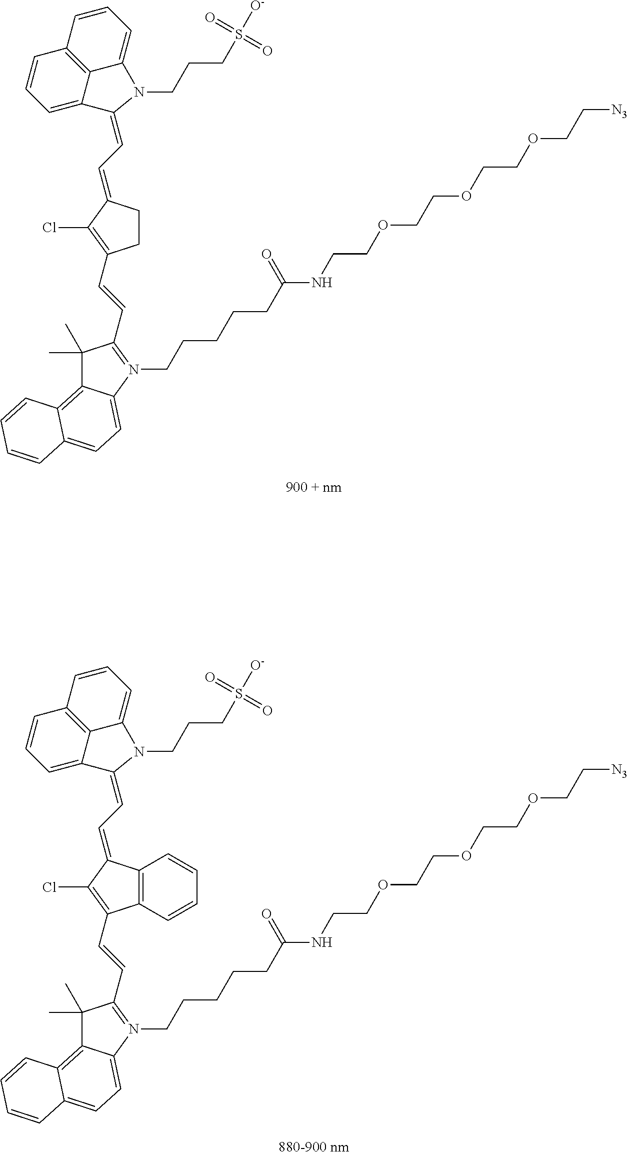

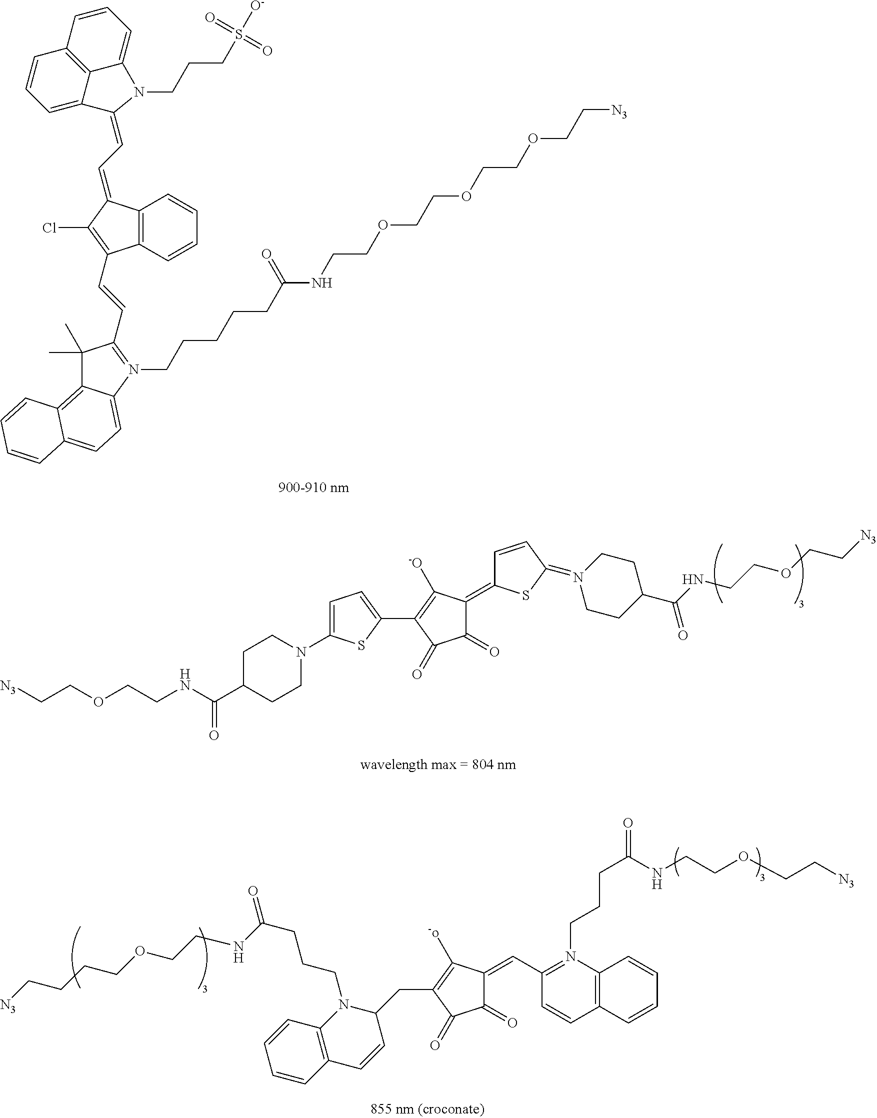

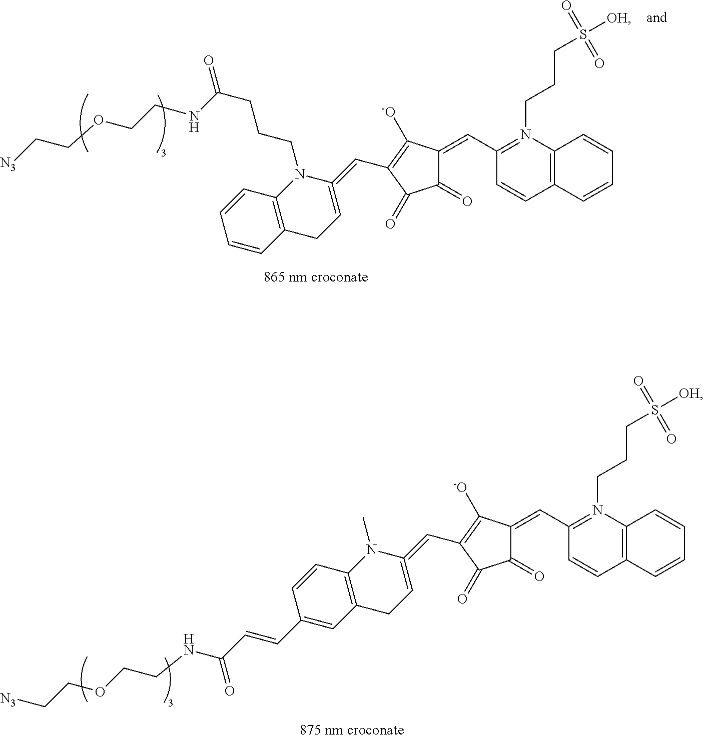

The skilled artisan will appreciate that while each of the exemplified compounds includes an azide group (i.e. N₃), that another functional group capable of participating in a “click chemistry” reaction may be substituted for the azide group, including any of the click functional groups listed in Table 1 below:

TABLE 1
Reactive functional groups capable of
participating in a click chemistry reaction.
Reactive Functional Groups Capable of
Particpating in a Click Chemistry Reaction

Alkyne
Azide
diarylcyclooctyne (“DBCO”)
Alkene
Trans-cyclooctene (“TCO”)
Maleimide
DBCO
Aldehyde or ketone
Tetrazine
Thiol
1,3-Nitrone
Hydrazine
Hydroxylamine
Tetrazine

In some embodiments, the detectable conjugates are selected from the following:

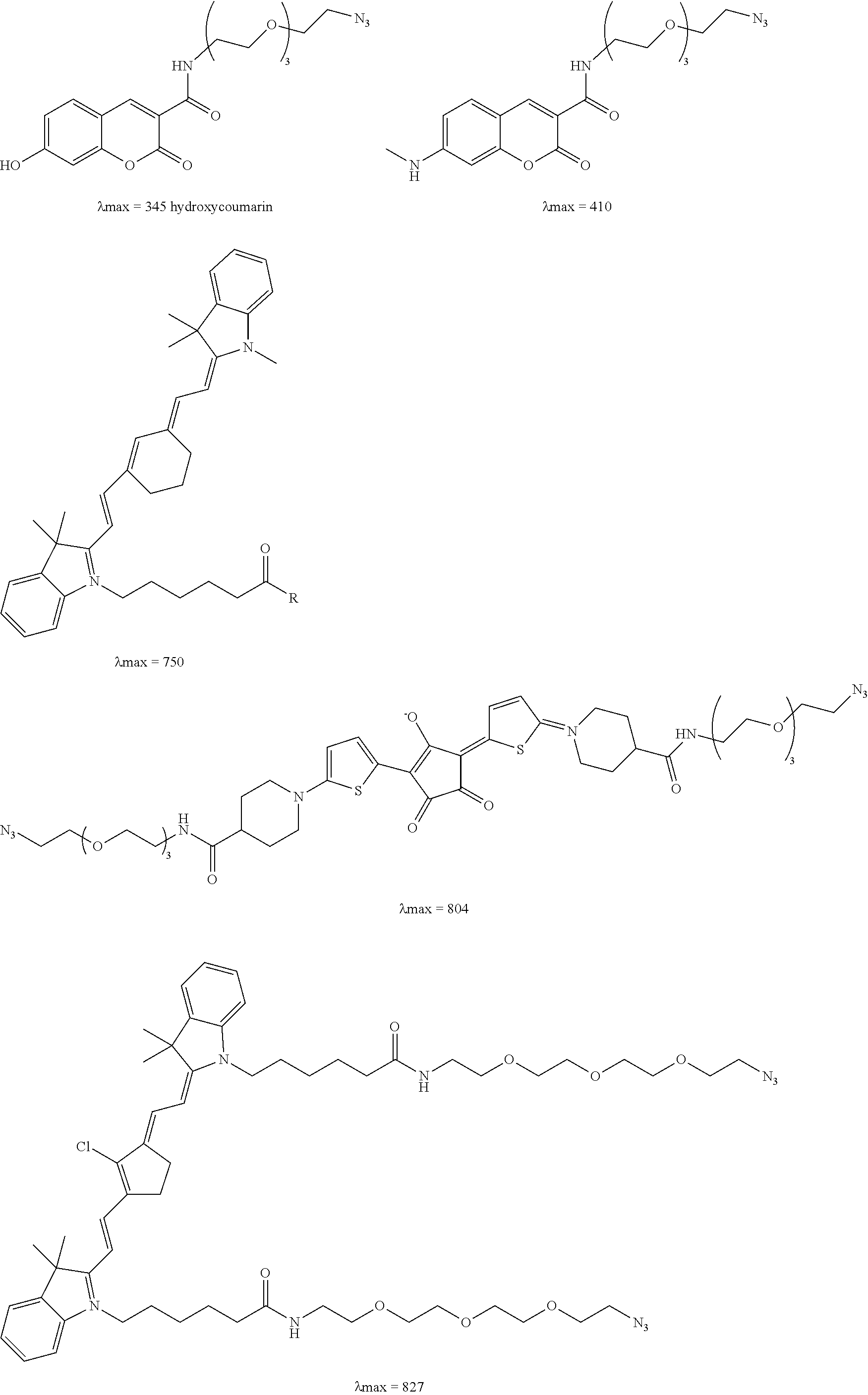

Methods

The present disclosure is also directed to methods of staining a biological specimen with one or more conventional dyes (either “routine stains” or “special stains”) and further labeling one or more biomarkers within the biological specimen with one or more detectable moieties. In some embodiments, the one or more conventional dyes are detectable within the visible spectrum. For instance, the one or more conventional dyes have peak absorbance wavelengths ranging from between about 400 nm to about 700 nm. In some embodiments, the one or more detectable moieties are detectable outside the visible spectrum, e.g., within the ultraviolet spectrum or within the infrared spectrum. In some embodiments, at least two biomarkers are labeled with two different detectable moieties. In other embodiments, at least three biomarkers are labeled with three different detectable moieties. In yet other embodiments, at least four biomarkers are labeled with four different detectable moieties. In further embodiments, five or more biomarkers are labeled with five or more different detectable moieties.

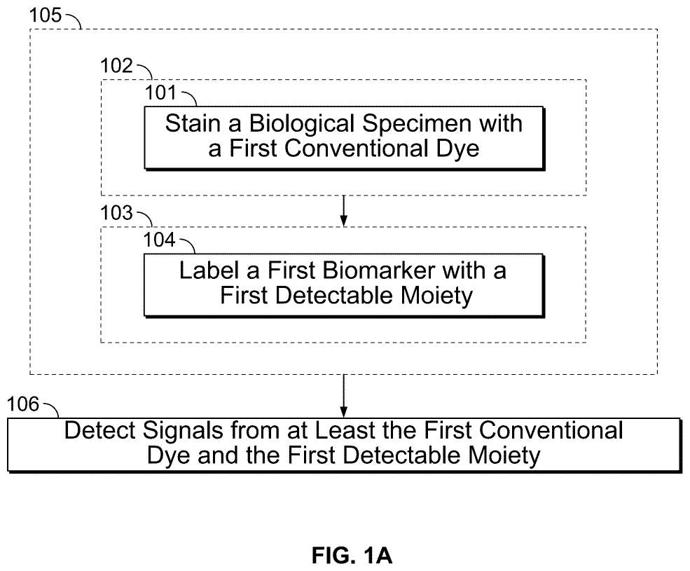

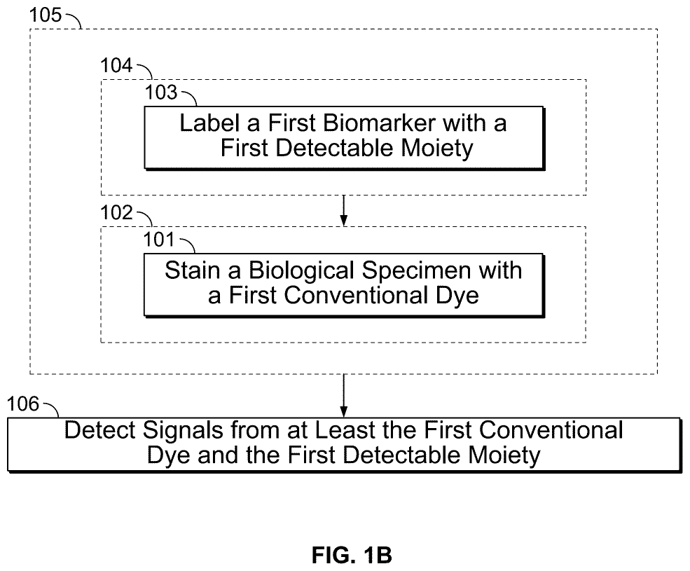

With reference to FIG. 1A, in some embodiments, a biological specimen is stained with a first conventional dye (step 101). In some embodiments, the first conventional dye is selected from hematoxylin, eosin, Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red O, Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain (1908), or any combination thereof.

In some embodiments, step 101 may be repeated a plurality of times (step 102) with different conventional dyes. In this manner, a biological specimen may be stained with one or more conventional dyes. For example, the biological specimen may be stained with hematoxylin and eosin. In some embodiments, the one or more conventional dyes are detectable within the visible spectrum. For instance, the one or more conventional dyes have peak absorbance wavelengths ranging from between about 400 nm to about 700 nm.

Next, a first biomarker is labeled with a first detectable moiety (step 103), where the first detectable moiety produces signals that are detectable outside the visible spectrum. In some embodiments, the first detectable moiety is detectable within the ultraviolet spectrum or within the infrared spectrum. In some embodiments, the first detectable moiety includes detectable moieties having a coumarin core, heptamethine cyanine core, or a croconate core.

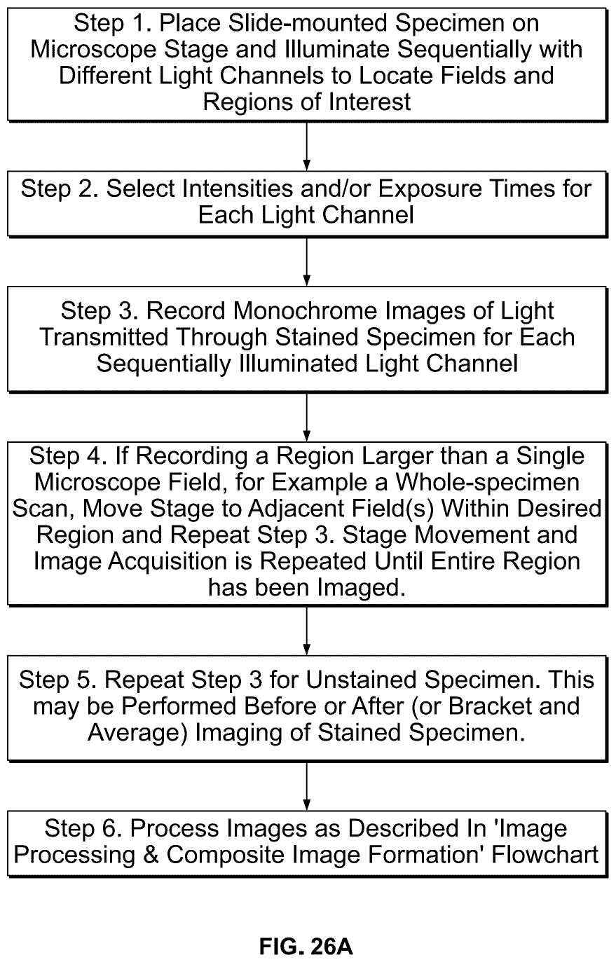

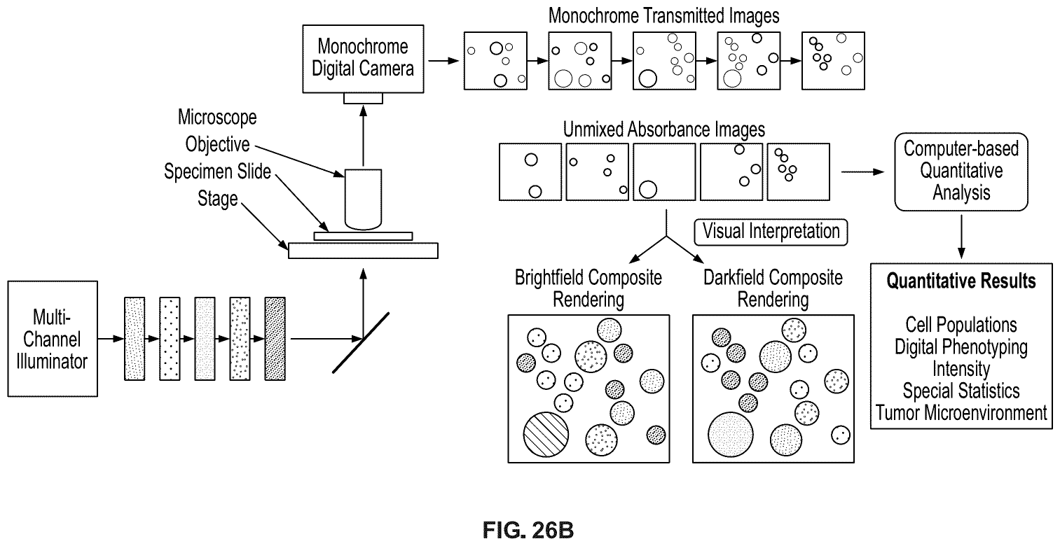

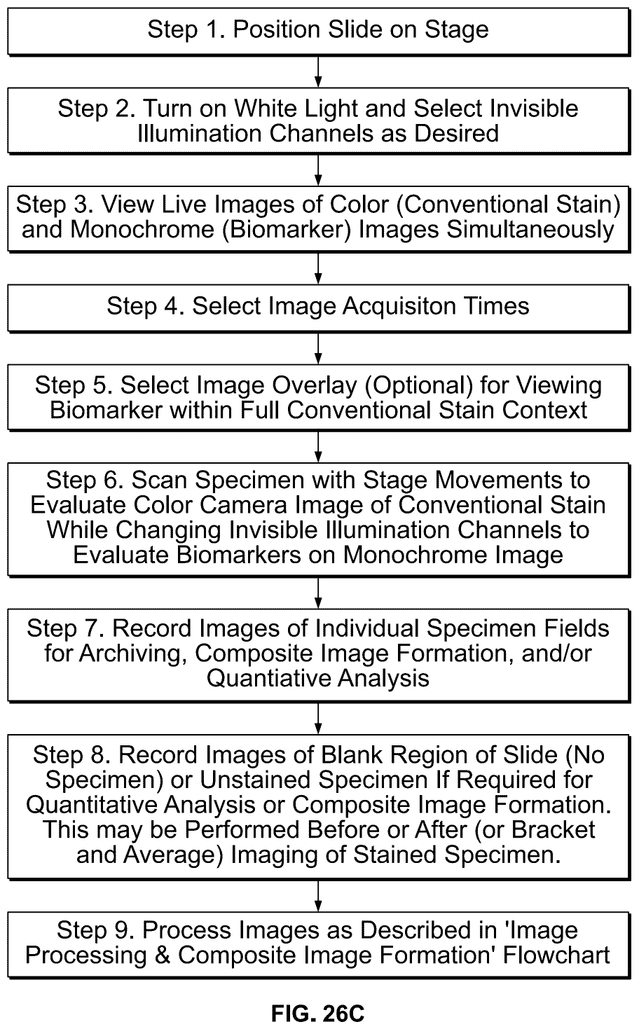

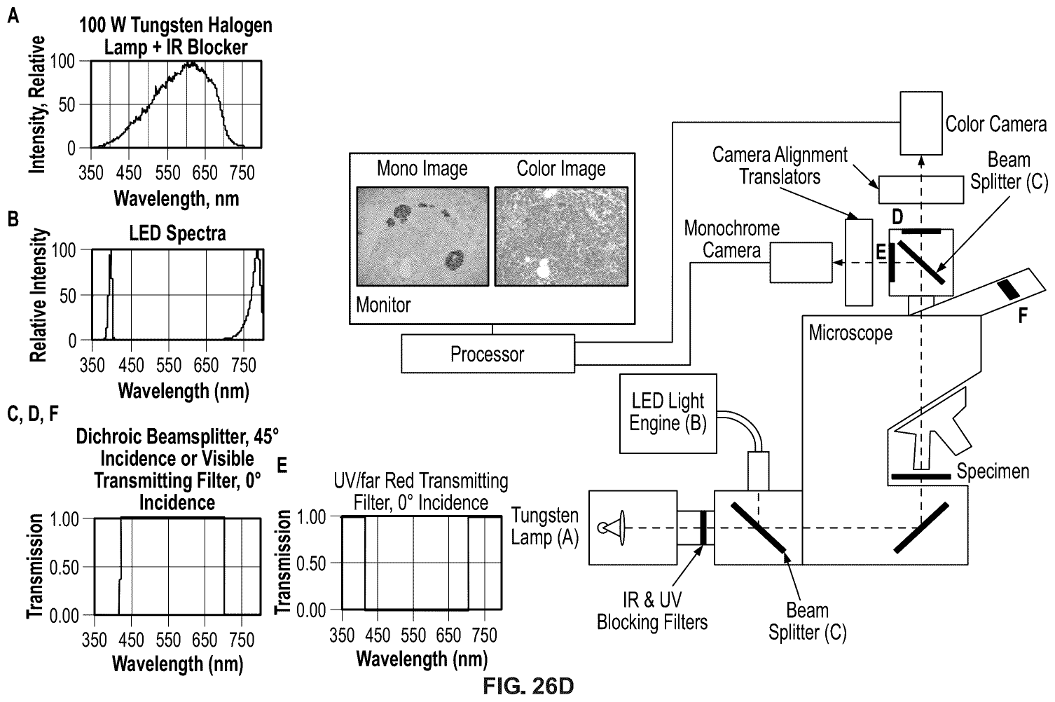

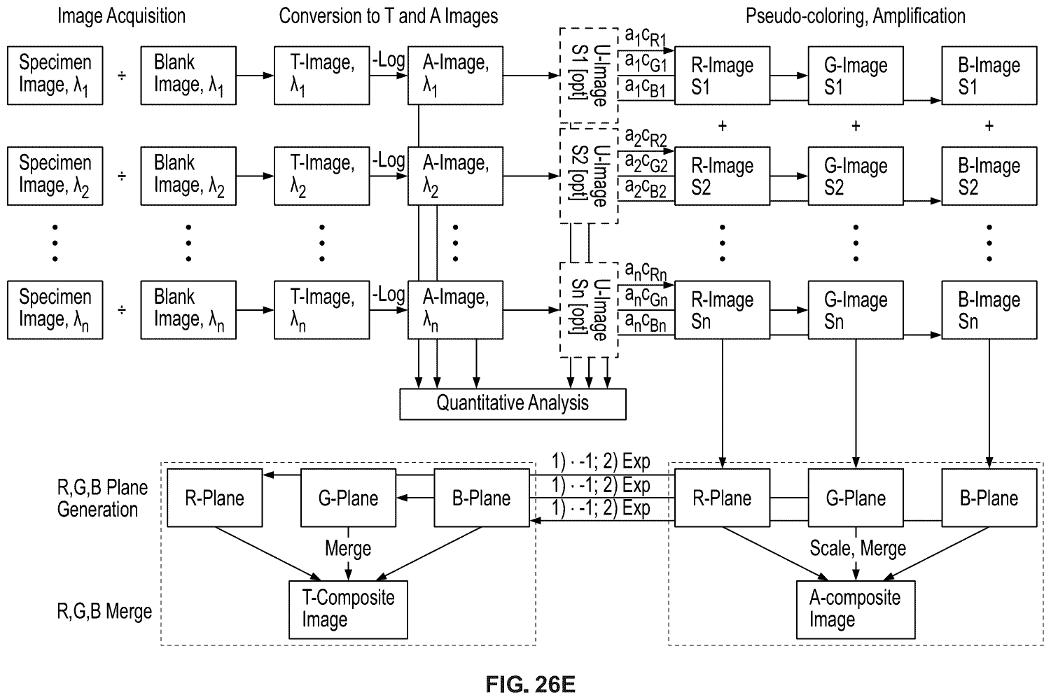

In some embodiments, step 103 is repeated a plurality of times (step 104) to label one or more biomarkers with one or more detectable moieties, where each of the one or more detectable moieties are different from each other, and wherein each of the one or more detectable moieties are outside the visible spectrum. In some embodiments, steps 103 and 104 may also be repeated as needed (step 105). Subsequently, the signals of the one or more conventional dyes and the one or more detectable moieties are detected (step 106). FIGS. 26A-26E illustrate methods of detecting and unmixing signals. In some embodiments, a composite image of the biological specimen is generated by combining the one or more signals from the one or more conventional dyes and the one or more signals from the one or more detectable moieties. In some embodiments, the one or more signals from the one or more detectable moieties within the generated image include false colors.

In some embodiments, steps 103 or 104 are performed first; and steps 101 and 102 are performed subsequently (see, e.g., FIG. 1B). In other embodiments, steps 101 and 103 are performed sequentially, and then both steps 101 and 103 are repeated one or more additional times. In yet other embodiments, steps 101 and 103 are performed simultaneously.

In those embodiments where one or more biomarkers are labeled with one or more detectable moieties (e.g. detectable moieties detectable outside the visible spectrum), each of the one or more detectable moieties are selected such that each of detectable moieties have (i) different peak absorbance wavelengths, (ii) have peak absorbance wavelengths that are outside the visible spectrum (e.g. have peak absorbance wavelengths of less than about 430 nm or have peak absorbance wavelengths of greater than 670 nm), and/or (iii) have peak absorbance wavelengths that do not substantially overlap (e.g. the different peak absorbance wavelengths differ by at least by at least 30 nm, by at least 40 nm, by at least 50 nm, by at least 60 nm, by at least 70 nm, by at least 80 nm, by at least 90 nm, by at least 100 nm, by at least 110 nm, by at least 120 nm, by at least 130 nm, by at least 140 nm, by at least 150 nm, by at least 170 nm, by at least 190 nm, by at least 210 nm, by at least 230 nm, by at least 250 nm, by at least 270 nm, by at least 290 nm, by at least 310 nm, etc.). In some embodiments, the detectable moieties are further selected such that their peak absorbance wavelengths do not overlap with the peak absorbance wavelengths of the conventional dyes applied to the biological specimen (regardless of where in the spectrum the peak absorbance wavelengths occurs). For example, first and second biomarkers may be labeled with first and second detectable moieties, where the first and second detectable moieties are different, are both outside the visible spectrum, and whose peak absorbance wavelengths do not substantially overlap. In some embodiments, first and second biomarkers may be labeled with first and second detectable moieties, where the first and second detectable moieties are different, are both outside the visible spectrum, and whose peak absorbance wavelengths differ by at least 20 nm, 30 nm, 50 nm, 60 nm, etc.

In some embodiments, each of the one or more detectable moieties have different peak absorbance wavelengths, wherein the different peak absorbance wavelengths of each of the one or more detectable moieties are separated by at least 20 nm, and wherein each of the one or more detectable moieties have FWHM of less than about 200 nm. In some embodiments, each of the one or more detectable moieties have different peak absorbance wavelengths, wherein the different peak absorbance wavelengths of one or more detectable moieties are separated by at least and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 200 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 150 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 100 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 70 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 70 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 70 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 70 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 50 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 50 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 50 nm.

Two methods of labeling one or more biomarkers in a biological specimen (such as a biological specimen that is pre-stained with a conventional dye, or stained subsequently with a conventional dye) are described herein. The first method utilizes detectable moieties (including any of those described herein) conjugated to a tyramide or quinone methide moiety (either directly or indirectly through one or more linkers). The second method utilizes detectable moieties (including any of those described herein) conjugated (either directly or indirectly through one or more linkers) to a reactive functional group capable of participating in a click chemistry reaction. Methods and reagents for detecting targets in biological specimens using tyramide chemistry, quinone methide chemistry, and click chemistry are described in U.S. Pat. No. 10,041,950, and in U.S. Publication Nos. 2019/0204330, 2017/0089911, and 2019/0187130, the disclosures of which are hereby incorporated by reference herein in their entireties.

In both methods, one or more biomarkers in the biological specimen are first labeled with an enzyme. Said another way, a first step in either method is forming one or more biomarker-enzyme complexes. In some embodiments, the one or more biomarker-enzyme complexes serve as intermediates for further reaction in either of the two methods described herein. Suitable enzymes for labeling the one or more biomarkers include, but are not limited to, horseradish peroxidase (HRP), alkaline phosphatase (AP), acid phosphatase, glucose oxidase, β-galactosidase, β-glucuronidase or β-lactamase. In some embodiments, the one or more biomarkers are labeled with horseradish peroxidase or alkaline phosphatase. In some embodiments, the one or more biomarkers are each labeled with the same enzyme. In other embodiments, the one or more biomarkers are labeled with different enzymes.

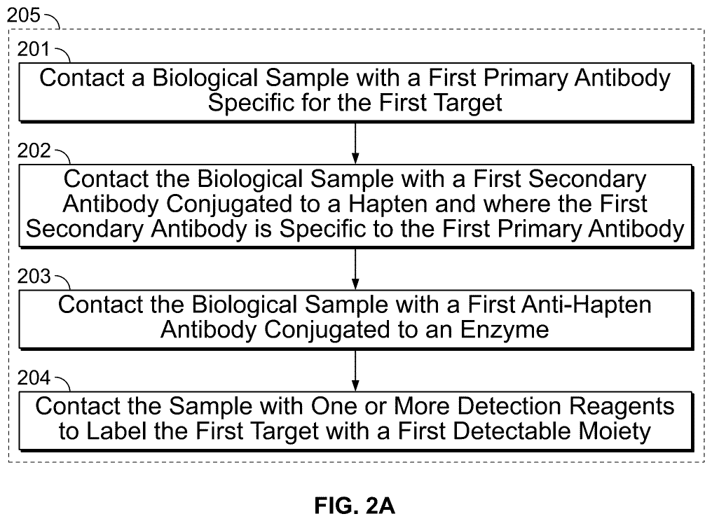

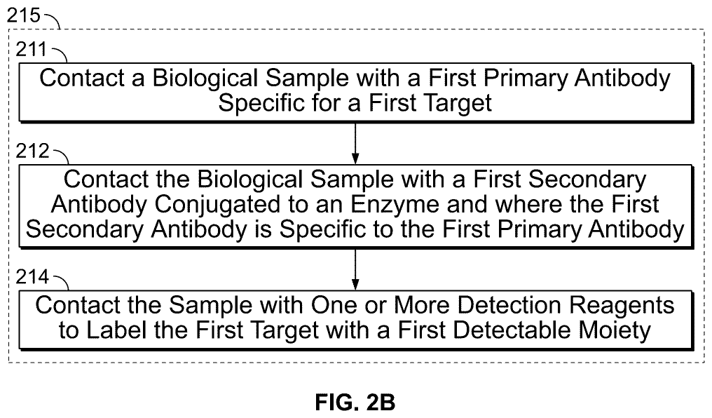

To facilitate the labeling of the one or more biomarkers with an enzyme, in some embodiments, one or more specific binding entities specific to the one or more biomarkers are introduced to the biological specimen. With reference to FIGS. 2A and 2B, in some embodiments the one or more specific binding entities specific to the one or more biomarkers are primary antibodies (step 201, 211). Following introduction of the primary antibodies, one or more secondary antibodies each conjugated to a label (directly or indirectly through a linker) may be introduced, where the secondary antibodies are specific to the primary antibodies (e.g. the secondary antibody is an anti-primary antibody antibody) (steps 202, 212). In some embodiments, the label of each of the secondary antibodies is an enzyme, including any of those described above (see step 212 of FIG. 2B).

In other embodiments, the label of the one or more secondary antibodies are haptens (see step 202 of FIG. 2A). Non-limiting examples of haptens include an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, 2,3,6,7-tetrahydro-11-oxo-1H,5H,11H-[1]benzopyrano[6,7,8-ij]quinolizine-10-carboxylic acid, or 7-diethylamino-3-carboxycoumarin. Other suitable haptens are disclosed in U.S. Pat. No. 8,846,320, the disclosure of which is hereby incorporated by reference herein in its entirety. In those embodiments where the secondary antibody is conjugated to a hapten, an anti-hapten antibody conjugated to an enzyme (including any of those described above) may be introduced to the biological specimen to label the target with one or more enzymes (step 203). Subsequently, suitable detection reagents may be introduced to the biological specimen to facilitate the labeling of the one or more biomarkers (now coupled indirectly to an enzyme) with one or more detectable moieties (including any of the detectable moieties described herein) (steps 204, 214). The steps in FIGS. 2A and 2B may be repeated any number of times (see steps 205 and 215).

In some embodiments, the one or more specific binding entities are primary antibody conjugates and/or nucleic acid probe conjugates. In some embodiments, the one or more specific binding entities are primary antibody conjugates coupled to an enzyme. In some embodiments, the primary antibody conjugates are conjugated to horseradish peroxidase or alkaline phosphatase. In other embodiments, the one or more specific binding entities are nucleic acid probes conjugated to an enzyme, e.g. horseradish peroxidase or alkaline phosphatase. Introduction of the one or more specific binding entities conjugated to an enzyme facilitates the formation of one or more biomarker-enzyme complexes.

In some embodiments, the one or more specific binding entities are primary antibody conjugates coupled to a hapten and/or one or more nucleic acid probes conjugated to a hapten (including any of those haptens described in U.S. Pat. No. 8,846,320, the disclosure of which is hereby incorporated by reference herein in its entirety). In these embodiments, the introduction of the one or more specific binding entities conjugated to haptens facilitates for the formation of one or more hapten-labeled biomarkers. In these embodiments, one or more anti-hapten antibody-enzyme conjugates specific to the haptens of the one or more hapten-labeled biomarkers are introduced to the biological specimen so as to label the one or more hapten-labeled biomarkers with an enzyme to provide one or more biomarker-enzyme complexes. The primary antibody conjugates, secondary antibodies, and/or nucleic acid probes may be introduced to a sample according to procedures known to those of ordinary skill in the art to effect labeling of one or more targets in a biological specimen with an enzyme and as illustrated herein.

Each of the aforementioned methods are described in more detail herein, including the steps of staining the biological specimen with one or more conventional dyes (where the staining with the conventional dyes may take place either before or after the labeling of the one or more biomarkers with one or more detectable moieties).

Methods of Detecting One or More Labeled Biomarkers in a Biological Specimen Using Tyramide and/or Quinone Methide Conjugates, where the Biological Specimen is Stained with One or More Conventional Dyes

The present disclosure is also directed to methods of (i) staining a biological specimen with one or more conventional dyes detectable within the visible spectrum, including any of the “routine stains” or “special stains” described herein, and (ii) labeling one or more biomarkers within the biological specimen with one or more detectable conjugates, where each of the detectable conjugates comprises (i) a tyramide and/or quinone methide moiety, and (ii) a detectable moiety having a peak absorbance wavelength that is outside the visible spectrum. In some embodiments, the one or more detectable moieties have peak absorbance wavelengths within either the ultraviolet spectrum or the infrared spectrum. In some embodiments, the one or more detectable moieties each have a peak absorbance wavelength less than about 430 nm or greater than about 670 nm.

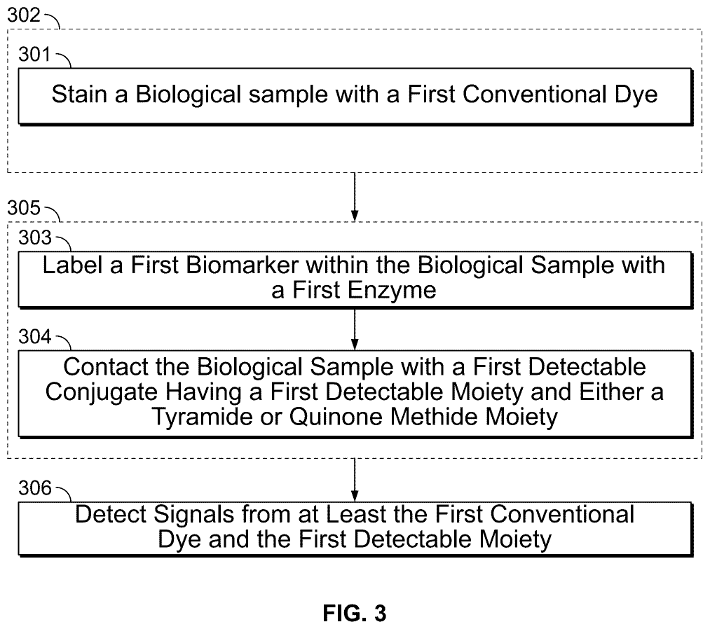

In some embodiments, and with reference to FIG. 3, a biological specimen is first stained with a first conventional dye (step 301). Step 301 may be repeated one or more times to provide a biological specimen stained with one or more conventional dyes. For example, the biological specimen may be stained with both hematoxylin and eosin. In some embodiments, the one or more conventional dyes are routine stains. In some embodiments, the one or more conventional dyes are special stains. In some embodiments, the one or more conventional dyes are selected from hematoxylin, eosin, Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red O, Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, and Wright stain (1908). In some embodiments, the one or more conventional dyes are hematoxylin and eosin.

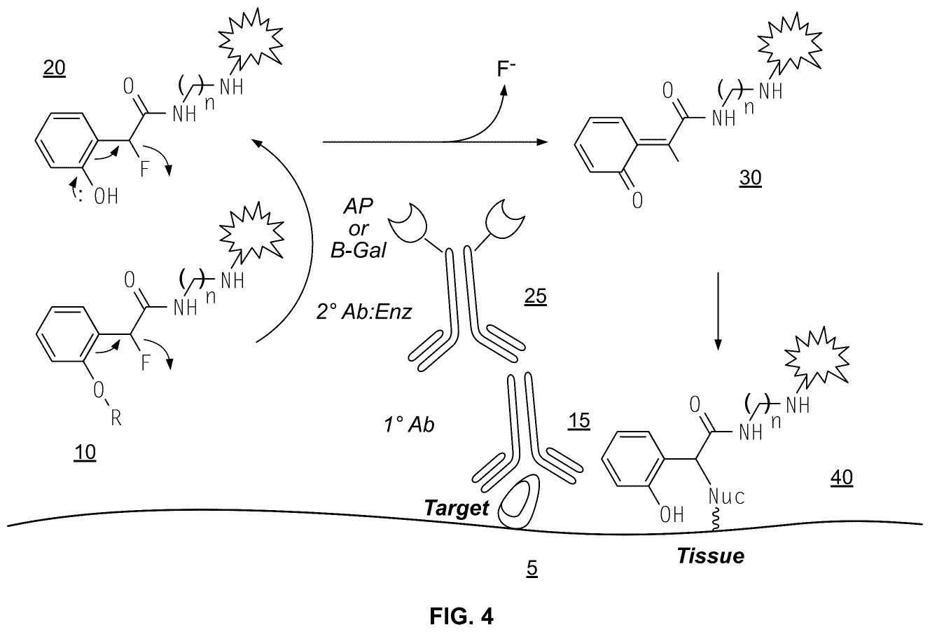

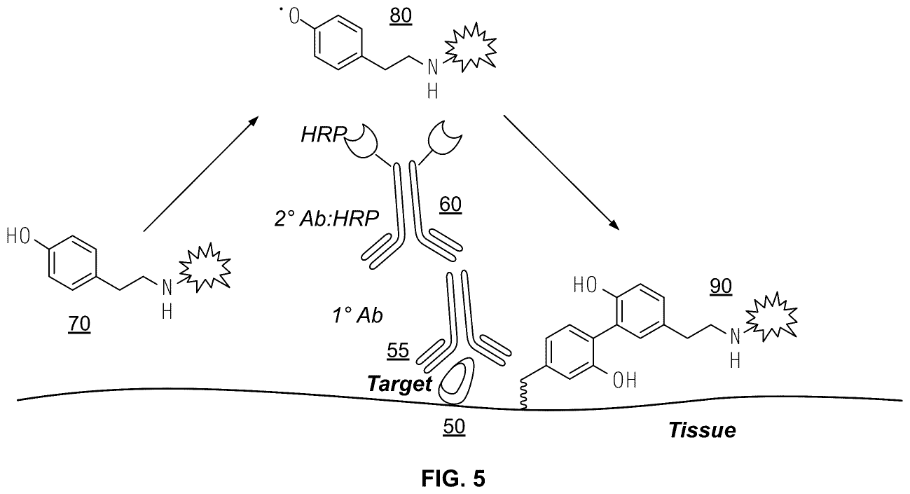

Next, a first biomarker within the biological specimen is labeled with a first enzyme (step 303) to form a first biomarker-enzyme complex. Methods of labeling a first biomarker with a first enzyme are described above and also illustrated in FIGS. 2A and 2B. The biological specimen including the first biomarker-enzyme complex is then contacted with a first detectable conjugate (step 304), the first detectable conjugate comprising a first detectable moiety having a peak absorbance wavelength that is outside the visible spectrum (including any of those described herein) and either a tyramide, a quinone methide, or a derivative or analog thereof. Upon interaction of the first enzyme of the first biomarker-enzyme complex with the tyramide or the quinone methide portion of the first detectable conjugate, at least the first detectable moiety of the first detectable conjugate is deposited proximal to or onto the first biomarker target (see also FIGS. 4 and 5 which illustrate the deposition of a detectable moiety proximal to or onto a target molecule within a biological specimen, where the target molecule 5 or 50 may be a biomarker).

The steps of labeling a biomarker with an enzyme (step 303) and subsequently a detectable moiety (step 304) may be repeated (step 305) any number of times and for any different types of biomarkers (e.g. protein, nucleic acid) within the biological specimen. In some embodiments, each biomarker within the biological specimen is labeled with a different detectable moiety. For example, a Ki-67 biomarker may be labeled with a detectable moiety having a FWHM of less than 50 nm and a peak absorbance wavelength between 330 nm and 390 nm; and a PD-L1 biomarker may be labeled with a detectable moiety having a FWHM of less than 50 nm and a peak absorbance wavelength between 760 nm and 820 nm.

Finally, signals of the one or more conventional dyes and the one or more detectable moieties are detected (step 106). FIGS. 26A-26E illustrate methods of detecting and unmixing signals. In some embodiments, the one or more detectable moieties are detected by illuminating the biological specimen with light at a wavelength specific to the detectable moiety. Methods of detecting one or more signals from one or more detectable moieties are described in PCT Application No. WO/2014/143155, the disclosure of which is hereby incorporated by reference herein in its entirety and described further herein. In some embodiments, a composite image of the biological specimen is generated by combining the one or more signals from the one or more conventional dyes and the one or more signals from the one or more detectable moieties. In some embodiments, the one or more signals from the one or more detectable moieties within the generated image include false colors (see, e.g., FIG. 26E).

In those embodiments where one or more biomarkers are labeled with one or more detectable moieties (e.g. detectable moieties detectable outside the visible spectrum), each of the one or more detectable moieties are selected such that each of detectable moieties have (i) different peak absorbance wavelengths, (ii) have peak absorbance wavelengths that are outside the visible spectrum (e.g. have peak absorbance wavelengths of less than about 430 nm or have peak absorbance wavelengths of greater than 670 nm), and/or (iii) have peak absorbance wavelengths that do not substantially overlap (e.g. the different peak absorbance wavelengths differ by at least 20 nm, by at least 30 nm, by at least 40 nm, by at least 50 nm, by at least 60 nm, by at least 70 nm, by at least 80 nm, by at least 90 nm, by at least 100 nm, by at least 110 nm, by at least 120 nm, by at least 130 nm, by at least 140 nm, by at least 150 nm, by at least 170 nm, by at least 190 nm, by at least 210 nm, by at least 230 nm, by at least 250 nm, by at least 270 nm, by at least 290 nm, by at least 310 nm, etc.). In some embodiments, the detectable moieties are further selected such that their peak absorbance wavelengths do not overlap with the peak absorbance wavelengths of the conventional dyes applied to the biological specimen (regardless of where in the spectrum the peak absorbance wavelengths occurs). For example, first and second biomarkers may be labeled with first and second detectable moieties, where the first and second detectable moieties are different, are both outside the visible spectrum, and whose peak absorbance wavelengths do not substantially overlap. In some embodiments, first and second biomarkers may be labeled with first and second detectable moieties, where the first and second detectable moieties are different, are both outside the visible spectrum, and whose peak absorbance wavelengths differ by at least 20 nm, 30 nm, 50 nm, 60 nm, etc.

In some embodiments, each of the one or more detectable moieties have different peak absorbance wavelengths, wherein the different peak absorbance wavelengths of each of the one or more detectable moieties are separated by at least 20 nm, and wherein each of the one or more detectable moieties have FWHM of less than about 200 nm. In some embodiments, each of the one or more detectable moieties have different peak absorbance wavelengths, wherein the different peak absorbance wavelengths of one or more detectable moieties are separated by at least and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 200 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 150 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 100 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than 70 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than 70 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than 70 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than 70 nm.

FIGS. 4 and 5 further illustrate the reactions that take place between the various components introduced to the biological specimen. With reference to FIG. 4, a specific binding entity 15 is first introduced to a biological specimen having a target 5 to form a target-detection probe complex. In some embodiments, the target 5 is a biomarker and the formed target-detection probe complex is a biomarker-detection probe complex. In some embodiments, the specific binding entity 15 is a primary antibody. Subsequently, a labeling conjugate 25 is introduced to the biological specimen, the labeling conjugate 25 comprising at least one enzyme conjugated thereto. In the embodiment depicted, the labeling conjugate 25 is a secondary antibody, where the secondary antibody is an anti-species antibody conjugated to an enzyme. Next, a detectable conjugate 10 is introduced, such as a detectable conjugate including any of the detectable moieties described herein coupled directly or indirectly to a quinone methide moiety or a derivative or analog thereof. Upon interaction of the enzyme (e.g. AP or B-Gal) with the detectable conjugate 10, the detectable conjugate 10 undergoes a structural, conformational, or electronic change 20 to form a tissue reactive intermediate 30. In this particular embodiment, the detectable conjugate comprises a quinone methide precursor moiety that, upon interaction with the alkaline phosphatase enzyme (of the labeling conjugate 25), causes a fluorine leaving group to be ejected, resulting in the respective quinone methide intermediate 30. The quinone methide intermediate 30 then forms a covalent bond with the tissue proximal or directly on the tissue to form a detectable moiety complex 40. Signals from the detectable moiety complex 40 may then be detected according to methods known to those of ordinary skill in the art, such as those described in U.S. Pat. No. 10,041,950, and in U.S. Publication Nos. 2019/0204330, 2017/0089911, and 2019/0187130 and in PCT Publication No. WO/2014/143155, the disclosures of which are hereby incorporated by reference herein in its entirety. The steps of FIG. 4 may be repeated for one or more biomarkers within a target.

With reference to FIG. 5, a specific binding entity 55 is first introduced to a biological specimen having a target 50 to form a target-detection probe complex and the formed target-detection probe complex is a biomarker marker-detection probe complex. In some embodiments, the specific binding entity 55 is a primary antibody. Subsequently, a labeling conjugate 60 is introduced to the biological specimen, the labeling conjugate 60 comprising at least one enzyme conjugated thereto. In the embodiment depicted, the labeling conjugate is a secondary antibody, where the secondary antibody is an anti-species antibody conjugated to an enzyme. Next, a detectable conjugate 70 is introduced, such as a detectable conjugate including any of the detectable moieties described herein coupled directly or indirectly to a tyramide moiety or a derivative or analog thereof. Upon interaction of the enzyme with the detectable conjugate a tissue reactive intermediate 80 is formed. In this particular embodiment, the detectable conjugate 70 comprises a tyramide moiety that, upon interaction with horseradish peroxidase enzyme, causes formation of the radical species 80. The radical intermediate 80 then forms a covalent bond with the tissue proximal or directly on the tissue to form a detectable moiety complex 90. Signals from the detectable moiety complex 90 may then be detected according to methods known to those of ordinary skill in the art, such as those described in U.S. Pat. No. 10,041,950 and in U.S. Publication Nos. 2019/0204330, 2017/0089911, and 2019/0187130 and in PCT Publication No. WO/2014/143155, the disclosures of which are hereby incorporated by reference herein in its entirety. The steps of FIG. 5 may be repeated for one or more biomarkers within a target.

In some embodiments, the biological specimens are pre-treated with an enzyme inactivation composition to substantially or completely inactivate endogenous peroxidase activity. For example, some cells or tissues contain endogenous peroxidase. Using an HRP conjugated antibody may result in high, non-specific background staining. This non-specific background can be reduced by pre-treatment of the sample with an enzyme inactivation composition as disclosed herein. In some embodiments, the samples are pre-treated with hydrogen peroxide only (about 1% to about 3% by weight of an appropriate pre-treatment solution) to reduce endogenous peroxidase activity. Once the endogenous peroxidase activity has been reduced or inactivated, detection kits may be added, followed by inactivation of the enzymes present in the detection kits, as provided above. The disclosed enzyme inactivation composition and methods can also be used as a method to inactivate endogenous enzyme peroxidase activity. Additional inactivation compositions are described in U.S. Publication No. 2018/0120202, the disclosure of which is hereby incorporated by reference herein in its entirety.

In some embodiments if the specimen is a sample embedded in paraffin, the sample can be deparaffinized using appropriate deparaffinizing fluid(s). After a waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

Other conjugates for use in the methods of the present disclosure are set forth within U.S. Pat. Nos. 8,658,389 and 8,686,122, the disclosures of which are hereby incorporated by reference herein in their entireties. For instance, U.S. Pat. No. 8,658,389 discloses conjugates that include an antibody covalently linked to a signal-generating moiety.

Methods of Detecting Targets in a Sample Using a Pair of Click Conjugates

The present disclosure is also directed to methods of (i) staining a biological specimen with one or more conventional stains detectable within the visible spectrum, including any of the “routine stains” or “special stains” described herein, and (ii) labeling one or more biomarkers within the biological specimen with a pair of click conjugates. In these assays, one member of a pair of click conjugates comprises a detectable conjugate comprising: (i) a first functional group capable of participating in a click chemistry reaction, and (ii) a detectable moiety, including any of the detectable moieties described herein. Non-limiting examples of suitable detectable conjugates are described herein. Another member of the pair of click conjugates (hereinafter referred to as “tissue reactive conjugates”) comprises a conjugate comprising: (i) a tyramide moiety, a quinone methide moiety, or a derivative or analog of a tyramide moiety or a quinone methide moiety; and (ii) a second functional group capable of reacting the first functional group of the detectable conjugate. In some embodiments, the one or more detectable moieties have peak absorbance wavelengths within either the ultraviolet spectrum or the infrared spectrum. In some embodiments, the one or more detectable moieties each have a peak absorbance wavelength less than about 430 nm or greater than about 670 nm. Suitable first and second functional groups coupled to the detectable conjugate and the tissue reactive conjugate and capable of reacting with each other are set forth in Table 2:

TABLE 2
First and second functional groups capable of reacting
with each other in a “click chemistry” reaction.

| Reactive Functional Group | Reactive Functional Group |
|---|---|
| on a First Member of a | on a Second Member of a |
| Pair of Click Conjugates | Pair of Click Conjugates |
| Alkyne | Azide |
| Azide | Alkyne |
| diarylcyclooctyne (“DBCO”) | Azide |
| Alkene | Tetrazine |
| Trans-cyclooctene (“TCO”) | Tetrazine |
| Maleimide | Thiol |
| DBCO | 1,3-Nitrone |
| Aldehyde or ketone | Hydrazine |
| Aldehyde or ketone | Hydroxylamine |
| Azide | DBCO |
| Tetrazine | TCO |
| Thiol | Maleimide |
| 1,3-Nitrone | DBCO |
| Hydrazine | Aldehyde or ketone |
| Hydroxylamine | Aldehyde or ketone |
| Tetrazine | Alkene |

Non-limiting examples of suitable tissue reactive conjugates are illustrated below:

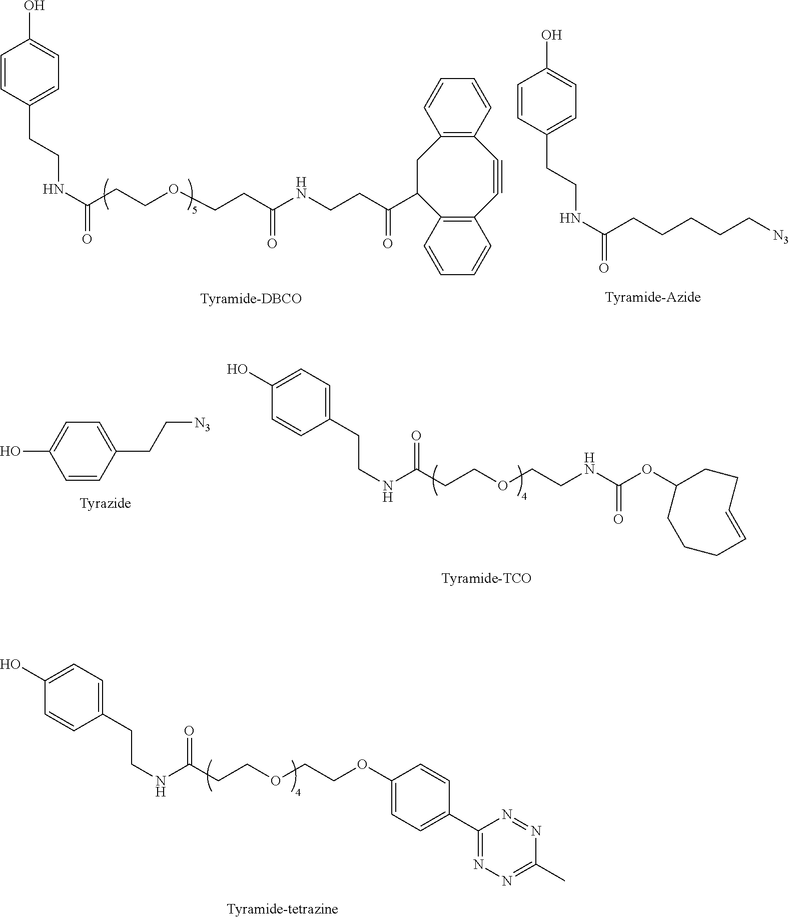

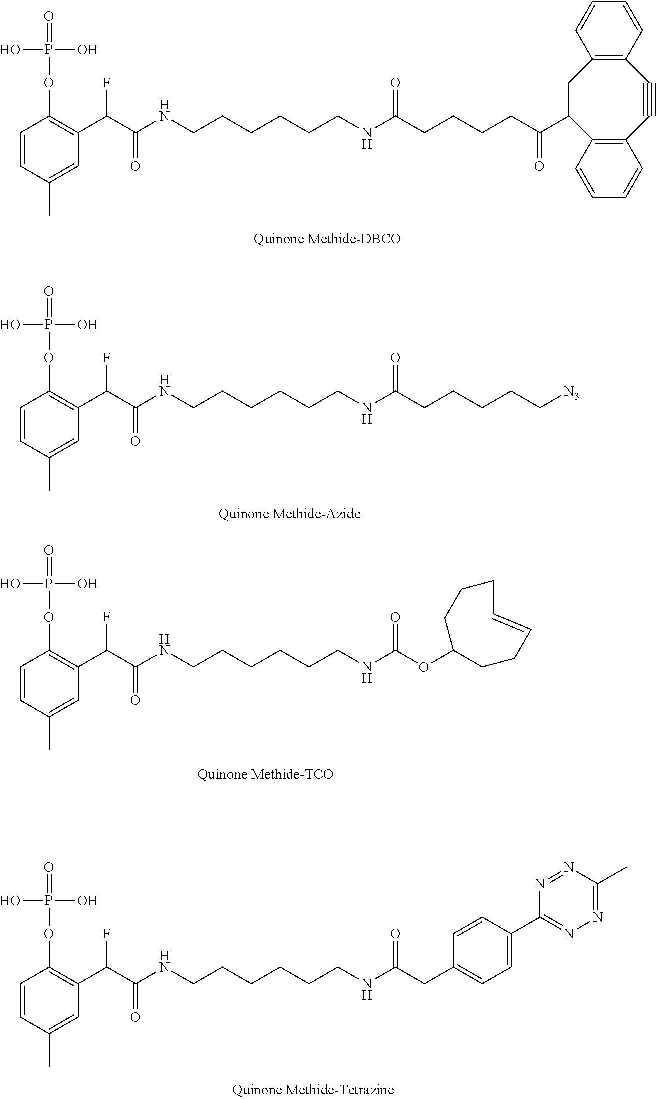 [SMILES: Cc1ccc(OP(=O)(O)O)c(C(F)C(=O)NCCCCCCNC(=O)CCCCC(=O)C2Cc3ccccc3CCc3ccccc32)c1 ; Cc1ccc(OP(=O)(O)O)c(C(F)C(=O)NCCCCCCNC(=O)CCCCCN=[N+]=[N-])c1 ; Cc1ccc(OP(=O)(O)O)c(C(F)C(=O)NCCCCCCNC(=O)OC2CC/C=C/CCC2)c1 ; Cc1ccc(OP(=O)(O)O)c(C(F)C(=O)NCCCCCCNC(=O)Cc2ccc(-c3nnc(C)nn3)cc2)c1]

Other suitable “tissue reactive conjugates” are described in U.S. Publication Nos. 2019/0204330, 2017/0089911, and 2019/0187130, the disclosures of which are hereby incorporated by reference herein their entireties.

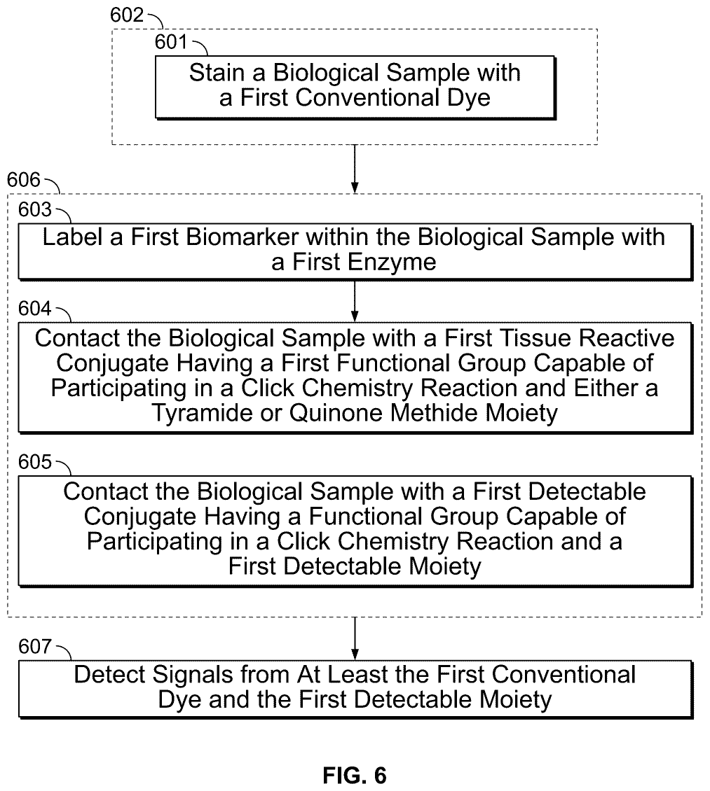

In some embodiments, and with reference to FIG. 6, a biological specimen is first stained with a first conventional dye (step 601). Step 601 may be repeated one or more times to provide a biological specimen stained with one or more conventional dyes (step 602). In some embodiments, the one or more conventional dyes are routine stains. In some embodiments, the one or more conventional dyes are special stains. In some embodiments, the one or more conventional dyes are selected from hematoxylin, eosin, Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red O, Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain (1908), and combinations thereof. In some embodiments, the one or more conventional dyes are hematoxylin and eosin.

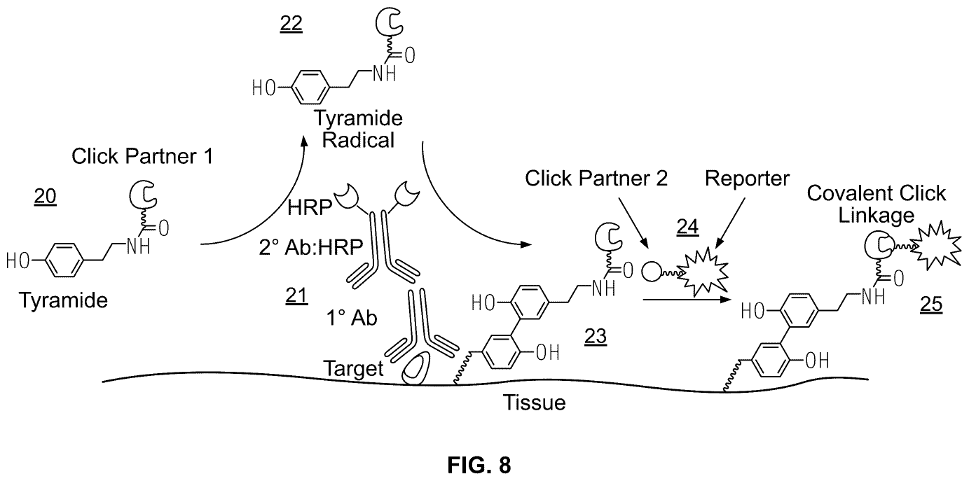

Subsequently, a first biomarker within the biological specimen is labeled with a first enzyme (step 603) to form a first biomarker-enzyme complex. Methods of labeling a first biomarker with a first enzyme are described above and also illustrated in FIGS. 2A and 2B. The biological specimen is then contacted with a first tissue reactive conjugate (step 604), the first tissue reactive conjugate comprising a first functional group capable of participating in a click chemistry reaction (including any of those described in Tables 1 and 2 herein) and either a tyramide, a quinone methide, or a derivative or analog thereof. Non-limiting examples of tissue reactive conjugates are provided herein. Upon interaction of the first enzyme of the first biomarker-enzyme complex with the tyramide or the quinone methide portion of the first tissue reactive conjugate, at least a first immobilized tissue-click conjugate complex is deposited proximal to or onto the first biomarker target (see also FIGS. 8 and 9 which further illustrate the “click chemistry” reactions that may take place and the formation of the resulting “first immobilized tissue-click conjugate complex” and “first immobilized tissue-click adduct complex”). Following the formation of the first immobilized tissue-click conjugate complex, the biological specimen is then contacted with a first detectable conjugate comprising: (i) a second functional group capable of reacting with the first reactive functional group of the first immobilized tissue-click conjugate complex, and (ii) a first detectable moiety (step 605), including any of the detectable moieties described herein. The reaction product of first immobilized tissue-click conjugate complex and first detectable conjugate produces a first immobilized tissue-click adduct complex which may be detected.

The aforementioned process (steps 603, 604, and 605) may be repeated (step 606) for any number of biomarkers within the biological specimen. In some embodiments, each biomarker is labeled with a different detectable moiety. For example, a Ki-67 biomarker may be labeled with a detectable moiety having a FWHM of less than 50 nm and a peak absorbance wavelength between 330 nm and 390 nm; and a PD-L1 biomarker may be labeled with a detectable moiety having a FWHM of less than 50 nm and a peak absorbance wavelength between 760 nm and 820 nm.

Finally, signals of the one or more conventional dyes and the one or more detectable moieties are detected (step 607). FIGS. 26A-26E illustrate methods of detecting and unmixing signals. In some embodiments, the one or more detectable moieties are detected by illuminating the biological specimen with light at a wavelength specific to the detectable moiety. Methods of detecting one or more signals from one or more detectable moieties are described in PCT Application No. WO/2014/143155, the disclosure of which is hereby incorporated by reference herein in its entirety and described further herein. In some embodiments, a composite image of the biological specimen is generated by combining the one or more signals from the one or more conventional dyes and the one or more signals from the one or more detectable moieties. In some embodiments, the one or more signals from the one or more detectable moieties within the generated image include false colors (see, e.g., FIG. 26E).

In those embodiments where one or more biomarkers are labeled with one or more detectable moieties (e.g. detectable moieties detectable outside the visible spectrum), each of the one or more detectable moieties are selected such that each of detectable have (i) different peak absorbance wavelengths, (ii) have peak absorbance wavelengths that are outside the visible spectrum (e.g. have peak absorbance wavelengths of less than about 430 nm or have peak absorbance wavelengths of greater than 670 nm), and/or (iii) have peak absorbance wavelengths that do not substantially overlap (e.g. the different peak absorbance wavelengths differ by at least 20 nm, by at least 30 nm, by at least 40 nm, by at least 50 nm, by at least 60 nm, by at least 70 nm, by at least 80 nm, by at least 90 nm, by at least 100 nm, by at least 110 nm, by at least 120 nm, by at least 130 nm, by at least 140 nm, by at least 150 nm, by at least 170 nm, by at least 190 nm, by at least 210 nm, by at least 230 nm, by at least 250 nm, by at least 270 nm, by at least 290 nm, by at least 310 nm, etc.). In some embodiments, the detectable moieties are further selected such that their peak absorbance wavelengths do not overlap with the peak absorbance wavelengths of the conventional dyes applied to the biological specimen (regardless of where in the spectrum the peak absorbance wavelengths occurs). For example, first and second biomarkers may be labeled with first and second detectable moieties, where the first and second detectable moieties are different, are both outside the visible spectrum, and whose peak absorbance wavelengths do not substantially overlap. In some embodiments, first and second biomarkers may be labeled with first and second detectable moieties, where the first and second detectable moieties are different, are both outside the visible spectrum, and whose peak absorbance wavelengths differ by at least 20 nm, 30 nm, 50 nm, 60 nm, etc.

In some embodiments, each of the one or more detectable moieties have different peak absorbance wavelengths, wherein the different peak absorbance wavelengths of each of the one or more detectable moieties are separated by at least 20 nm, and wherein each of the one or more detectable moieties have FWHM of less than about 200 nm. In some embodiments, each of the one or more detectable moieties have different peak absorbance wavelengths, wherein the different peak absorbance wavelengths of one or more detectable moieties are separated by at least and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 200 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 200 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 150 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 150 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than about 100 nm. In some embodiments, one or more detectable moieties have different peak absorbance wavelengths, wherein the different peak absorbance wavelengths of one or more detectable moieties are separated by at least 70 nm, and wherein each of the one or more detectable moieties have FWHM of less than about 100 nm.

In some embodiments, one or more detectable moieties have different peak absorbance wavelengths, wherein the different peak absorbance wavelengths of one or more detectable moieties are separated by at least 20 nm, and wherein each of the one or more detectable moieties have FWHM of less than 70 nm. In some embodiments, one or more detectable moieties have different peak absorbance wavelengths, wherein the different peak absorbance wavelengths of one or more detectable moieties are separated by at least 30 nm, and wherein each of the one or more detectable moieties have FWHM of less than 70 nm. In some embodiments, one or more detectable moieties have different peak absorbance wavelengths, wherein the different peak absorbance wavelengths of one or more detectable moieties are separated by at least 40 nm, and wherein each of the one or more detectable moieties have FWHM of less than 30 nm. In some embodiments, one or more detectable moieties have different peak absorbance wavelengths, wherein the different peak absorbance wavelengths of one or more detectable moieties are separated by at least 50 nm, and wherein each of the one or more detectable moieties have FWHM of less than 70 nm.

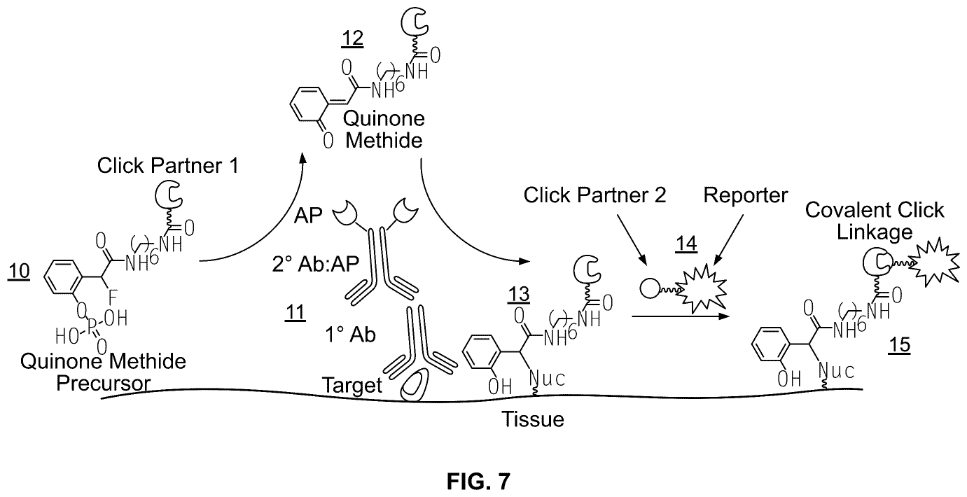

FIGS. 7 and 8 further illustrate the reaction between a first member of a pair of click conjugates having a tissue reactive moiety (10, 20) and a target-bound enzyme (11, 21) to form an immobilized tissue-click conjugate complex (13, 23). This first part of the amplification process is similar to that used in QMSA and TSA amplification processes. FIGS. 8 and 9 illustrate the subsequent reaction between the immobilized tissue-click conjugate (13, 23) complex and a second member of the pair of click conjugates (14, 24), to provide an immobilized tissue-click adduct complex (15, 25) comprising a detectable reporter moiety.

With reference to FIG. 7, a tissue reactive conjugate comprising a reactive functional group (10) is brought into contact with a target-bound enzyme (11) to produce a reactive intermediate (12). In some embodiments, the target-bound enzyme (11) is a biomarker-bound enzyme. In this example, the reactive intermediate, a quinone methide, forms a covalent bond to a nucleophile on or within a biological specimen, thus providing an immobilized tissue-click conjugate complex (13). The immobilized tissue-click conjugate complex may then react with a detectable conjugate having any of the detectable moieties described herein (14), provided that the tissue reactive conjugate 10 and the detectable conjugate 14 possess reactive functional groups that may react with each other to form a covalent bond. The reaction product of immobilized tissue-click conjugate complex 13 and click conjugate 14 produces the immobilized tissue-click adduct complex 15. The tissue-click adduct complex 15 may be detected by virtue of signals transmitted from the linked detectable moiety. In some embodiments, the steps of FIG. 7 may be repeated for any number of biomarkers.

Similarly, and with reference to FIG. 8, a tissue reactive conjugate comprising a reactive functional group (20) is brought into contact with a target-bound enzyme (21), to produce a reactive intermediate (22), namely a tyramide radical species (or derivative thereof). In some embodiments, the target-bound enzyme (21) is a biomarker-bound enzyme. The tyramide radical intermediate may then form a covalent bond to a biological specimen, thus providing an immobilized tissue-click conjugate complex (23). The immobilized tissue-click conjugate complex may then react with a detectable conjugate including any of the detectable moieties described herein (24), provided that tissue reactive conjugate and the detectable conjugate 20 and 24, respectively, possess reactive functional groups that may react with each other to form a covalent bond. The reaction product of immobilized tissue-click conjugate complex 23 and click conjugate 24 produces the tissue-click adduct complex 25. In some embodiments, the steps of FIG. 8 may be repeated for any number of biomarkers.

Automation

The assays and methods of the present disclosure may be automated and may be combined with a specimen processing apparatus. The specimen processing apparatus can be an automated apparatus, such as the BENCHMARK XT instrument and DISCOVERY XT instrument sold by Ventana Medical Systems, Inc. Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. Alternatively, specimens can be manually processed.

The specimen processing apparatus can apply fixatives to the specimen. Fixatives can include cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation).

If the specimen is a sample embedded in paraffin, the sample can be deparaffinized with the specimen processing apparatus using appropriate deparaffinizing fluid(s). After the waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

The specimen processing apparatus can apply a wide range of substances to the specimen. The substances include, without limitation, stains, probes, reagents, rinses, and/or conditioners. The substances can be fluids (e.g., gases, liquids, or gas/liquid mixtures), or the like. The fluids can be solvents (e.g., polar solvents, non-polar solvents, etc.), solutions (e.g., aqueous solutions or other types of solutions), or the like. Reagents can include, without limitation, stains, wetting agents, antibodies (e.g., monoclonal antibodies, polyclonal antibodies, etc.), antigen recovering fluids (e.g., aqueous- or non-aqueous-based antigen retrieval solutions, antigen recovering buffers, etc.), or the like. Probes can be an isolated nucleic acid or an isolated synthetic oligonucleotide, attached to a detectable label. Labels can include radioactive isotopes, enzyme substrates, co-factors, ligands, chemiluminescent or fluorescent agents, haptens, and enzymes.

Detection and/or Imaging

Certain aspects, or all, of the disclosed embodiments can be automated, and facilitated by computer analysis and/or image analysis system. In some applications, precise color or fluorescence ratios are measured. In some embodiments, light microscopy is utilized for image analysis. Certain disclosed embodiments involve acquiring digital images. This can be done by coupling a digital camera to a microscope. Digital images obtained of stained samples are analyzed using image analysis software. Color or fluorescence can be measured in several different ways. For example, color can be measured as red, blue, and green values; hue, saturation, and intensity values; and/or by measuring a specific wavelength or range of wavelengths using a spectral imaging camera. The samples also can be evaluated qualitatively and semi-quantitatively. Qualitative assessment includes assessing the staining intensity, identifying the positively-staining cells and the intracellular compartments involved in staining, and evaluating the overall sample or slide quality. Separate evaluations are performed on the test samples and this analysis can include a comparison to known average values to determine if the samples represent an abnormal state.

Suitable detection methods are described in PCT Application No. WO/2014/143155, the disclosure of which is hereby incorporated by reference herein in its entirety. In some embodiments, a suitable detection system comprises an imaging apparatus, one or more lenses, and a display in communication with the imaging apparatus. The imaging apparatus includes means for sequentially emitting energy and means for capturing an image/video. In some embodiments, the means for capturing is positioned to capture specimen images, each corresponding to the specimen being exposed to energy. In some embodiments, the means for capturing can include one or more cameras positioned on a front side and/or a backside of the microscope slide carrying the biological specimen. The display means, in some embodiments, includes a monitor or a screen. In some embodiments, the means for sequentially emitting energy includes multiple energy emitters. Each energy emitter can include one or more IR energy emitters, UV energy emitters, LED light emitters, combinations thereof, or other types of energy emitting devices. The imaging system can further include means for producing contrast enhanced color image data based on the specimen images captured by the means for capturing. The displaying means displays the specimen based on the contrast enhanced color image data.

Additional detection methods are illustrated in FIGS. 26A-26E, and further described herein.

### EXAMPLES

### Example 1. General Single and Multiplex IHC Procedure

Enzyme-antibody conjugates used with the detectable moieties were OmniMap anti-Ms HRP (RUO), DISCOVERY (VMSI Cat #760-4310), OmniMap anti-Rb HRP (RUO), DISCOVERY (VMSI Cat #760-4311), UltraMap anti-Ms Alk Phos, DISCOVERY (VMSI Cat #760-4312), and UltraMap anti-Rb Alk Phos, DISCOVERY (VMSI Cat #760-4314). Fully automated multiplexed detection was performed on a DISCOVERY Ultra system using the above primary antibodies and detection reagents. The DISCOVERY Universal Procedure was used to create a protocol for the single biomarker IHC and multiplex IHC. In general, IHC was performed at 37° C., unless otherwise noted, and reaction buffer wash solutions were diluted from 10×concentrate (cat. no. 950-300). A slide-mounted paraffin section was de-paraffinized by warming the slide to 70° C. for 3 cycles, each 8 min long. Antigen retrieval was performed by applying Cell Conditioning 1 (VMSI Cat. no. 950-124) and warming the slide to 94° C. for 64 min. Staining of each biomarker was performed in sequential steps that included incubation with primary antibody targeting that biomarker, washing in reaction buffer to remove unbound antibody, incubation with anti-species antibody targeting the primary antibody (either anti-mouse or anti-rabbit) conjugated to either peroxidase or alkaline phosphatase, depending on whether the chromogen is a tyramide or quinone methide derivative, respectively, washing with reaction buffer, incubation with tyramide-modified DBCO or tyramide-modified chromogenic reagent or quinone-methide-precursor-modified chromogenic reagent, and washing with reaction buffer. For tyramides, dilute H₂O₂ was added following tyramide addition to initiate the deposition. All deposition steps were followed by washing in reaction buffer. If tyramide-modified DBCO was used, the slide was further incubated with azide-modified chromogen, and washed. If multiplex IHC, before staining the next biomarker in sequence, the slide was incubated with Cell Conditioning 2 (VMSI Cat #950-123) at 100° C. for 8 min, followed by washing in reaction buffer. Finally, slides could be manually dehydrated through an ethanol series (2×80% ethanol, 1 min each, 2×90% ethanol, 1 min each, 3×100% ethanol, 1 min each, 3× xylene, 1 min each), at ambient temperature. Primary antibodies and enzyme-antibody conjugates were used at the concentrations, volumes, and incubation times recommended by the manufacturer. Tyramide-modified detectable conjugates (such as those described herein), tyramide-chromogen or tyramide-DBCO, were added to slides in 100 μL volumes at concentrations ranging between 25 and 1,200 μM in VMSI Discovery TSA diluent (cat no. 000060900). Azide-modified detectable conjugates were added as 100 μL volumes in TSA diluent typically at the same concentration as used for the tyramide-DBCO. The concentrations of solutions of the detectable conjugates (including the detectable moieties described herein) reflected their peak absorbance extinction coefficients, and biomarker expression levels, and were typically 1,200 μM for 7-amino-4-methylcoumarin-3-acetyl (AMCA), 400 μM for 7-hydroxycoumrin-3-carboxyl (HCCA), 600-800 μM for 7-diethylaminocoumarin-3-carboxyl (DCC), and 50-300 μM for Cy7 detectable moieties. Quinone-methide-precursor-modified Cy5 was added to slides in 100 μL of 400 μM Cy5 detectable moiety in TSA diluent.

### Example 2. Microscopy and Single-Camera Monochrome Imaging of Conventional Histological Staining and Detectable Moiety Staining

Multispectral imaging of H&E plus IHC specimens was performed on an Olympus BX-51 microscope (Olympus, Waltham, MA) fitted with a CoolSNAP ES2 CCD camera with a 1392×1040 pixel sensor at 12-bit resolution (Teledyne Photometrics, Tucson, AZ) and LED illumination, as previously described [Morrison L E, Lefever M R, Behman L J, Leibold T, Roberts E A, Horchner U B, Bauer D R. Brightfield Multiplex Immunohistochemistry with Multispectral Imaging. Lab Invest (2020) https://doi.org/10.1038/s41374-020-0429-0]. Microscope objectives were initially Olympus UPlanSApo 20× (NA 0.75) and 10× (NA 0.40) air objectives but were later updated with UPLXAPO 20× (NA 0.80) and UPLXAPO 10× (NA 0.4) objectives with improved chromatic aberration correction. Illumination was provided by a combination of optically filtered continuous light sources and LED illuminators. For the former, a Sutter Lambda 10-3 10-position filter wheel (Sutter Instruments, Novato, CA) was used with an Olympus 100 W tungsten halogen lamp to define up to nine wavelength channels. LED illumination was provided with a CoolLED (Andover, UK) pE-4000 16-channel illuminator and 2 Lumencor Spectra X light engines (Lumencor, Inc., Beaverton, OR), each containing 6 custom-selected LEDs. Illuminator outputs were focused onto 3 mm liquid light guides and the light guides combined into a single 3 mm diameter liquid light guide with one or two Lumencor combiners. The final light guide was connected to the illumination port of the microscope through a CoolLED pE collimator/microscope adapter. To reduce the illumination bandwidth further, each Lumencor LED was filtered with a single bandpass optical filter. Filter selection on the filter wheel and LED selection was achieved using manual controls with the option of computer control. Imaging of individual microscope fields on the CCD camera of light transmitted through the microscope was controlled by Micro-Manager software [Edelstein A D, Tsuchida M A, Amodaj N, Pinkard H, Wale R D, Stuuman N. Advanced methods of microscope control using μManager software. J Biol Methods 2014; 1:e10]. Image processing, including conversion between transmission and absorbance images, spectral unmixing, and formation of color composite images, was performed with ImageJ software [Schneider C A, Rasband W S, Eliceiri K W. NIH Image to ImageJ: 25 years of image analysis. Nat Methods 2012; 9: 671-675] or MATLAB software (Mathworks, Natick, MA, USA).

Typically, a multi-color specimen was imaged with multiple filters on the filter wheel and/or LED, where each filter and/or LED provided a band of light at wavelengths near the maximum absorbance of one of the dyes used to the stain the specimen (e.g. eosin and HTX or other conventional stains applied to the specimen, plus each detectable moiety). The number of different light channels utilized equaled at least the number of dyes. For calculation of transmission and absorbance images, an image was recorded using each light channel on an unstained region of the slide (e.g. to the side of the tissue/cellular specimen) before and/or after recording images with the same series of light channels at the desired region of interest within the stained specimen. Dividing transmitted light images of stained regions by images of unstained regions (100% transmission) provided transmittance (T) images. Logarithmic conversion provided absorbance (A) images (A=−log₁₀T) for which A is proportion to dye concentration according to Beer's Law. Color composite images were produced by addition of the monochrome A-images, with appropriate weighting for the desired pseudo-coloring, to form red, green, and blue color planes of the composite image. These composite images provided a “fluorescence-like” representation and can be converted to brightfield representations by anti-logarithmic conversion of A-images to T-images. Note that oculars are not required for multispectral imaging and may be replaced with a lens tube without oculars to avoid unintended exposure to bright light potentially used for imaging. If oculars are used, filters may be placed in the eyepieces that reduce transmitted light, such as blocking invisible light and transmitting only visible light (see Example 3 herein).

### Example 3. Microscopy and Dual-Camera Color/Monochrome Imaging of Conventional Histological Staining and Detectable Moiety Staining

FIG. 26D provides a schematic drawing of the dual-camera microscope system that permits simultaneous viewing of visible conventional stains and invisible IHC chromogens on a computer monitor. Olympus BX-51 and BX-63 microscopes (Olympus, Waltham, MA) were used with UPLXAPO 20× (NA 0.80) and UPLXAPO 10× (NA 0.4) objectives. Referring again to FIG. 26D, visible illumination was provided by an Olympus 100 W tungsten halogen lamp (A; Olympus U-LH100) with a hot mirror transmitting light between 420 nm and 690 nm (Newport Corp., Irvine, CA; cat. no. 10HMR-0), mounted in a Sutter Lambda 10-3 10-position filter wheel (Sutter Instruments, Novato, CA). A color-balancing filter (FGT165 filter, Thorlabs, Newton N J USA) may also be mounted after the hot mirror to enhance the blue end of the spectrum to improve direct viewing and color camera white balance. Far-blue/UV and far-red/near-IR light was provided by a second illumination source (B) comprising a pE-4000 16-channel LED illuminator (CoolLED (Andover, UK) and Lumencor Spectra X LED light engines (Lumencor, Inc., Beaverton, OR), or an additional Olympus 100 W tungsten halogen lamp, integral IR-blocking filter removed, combined with a filter wheel containing a collection of single bandpass filter sets. To further reduce illumination bandwidth, each Lumencor LED was filtered with a single bandpass optical filter. Some of the LED and filter characteristics are provided in the table set forth below. Visible illumination (A) and invisible illumination (B) were combined in a pE Combiner (CoolLED, Andover, UK) containing a reflective element (C), comprising either a 50-50 neutral density beamsplitter or a dichroic mirror with custom coating transmitting light between 420 and 700 nm and reflecting light below 420 nm and above 700 nm (Chroma Technology Corp., Bellows Falls, VT USA) oriented 45° to each illumination source. When LEDs were used, the invisible illumination source (B) could be expanded by combining multiple LED sources using 3 mm liquid light guides and one or more light guide combiners (Lumencor, Inc., Beaverton, OR USA) ahead of the pE-Combiner. After entering the brightfield illumination port of the microscope, the combined visible and invisible light passes through the specimen slide and microscope objective to the camera port and the dual-camera mount (2SCM1-DC; Thorlabs). A reflective element within the dual-camera mount, with the same reflective coating as the combiner reflective element (C), split invisible from visible light (when using the dichroic element), transmitting the visible light to the color camera (Kiralux CS505CU, Thorlabs), via a visible light transmitting filter (D; integral to camera) (which may also include a 435 nm long pass filter (Newport, 10CGA-435) when using the diethylcoumarin CDC to suppress yellow coloration). When a 50-50 neutral density mirror was used the reflective element (all wavelengths received at each camera), a 420 nm long pass filter is used with the integral color camera filter. Invisible light (when using the dichroic element) as reflected at the beamsplitter to the monochrome camera (Kiralux CS505MU, Thorlabs), via a filter (E) transmitting light below 420 nm and above 700 nm (custom ET560/280 notch filter, Chroma Technology Corp; when using the dichroic reflective element) or a Newport FSR-UG5 colored glass filter, transmitting light below 400 nm and above 690 nm (when using the 50-50 neutral density reflective element). Both cameras used the same underlying 2448×2048 pixel CMOS sensor allowing precise alignment of the two cameras using the translational and rotational adjustments within the 2-camera mount. Thorcam software (Thorlabs) provided control of live video from each camera, image overlays, and single frame image acquisition. In addition to the poor transmission of near UV light through the microscope optics, eye protection from invisible light was provided by custom barrier filters (F; ET560/280m, Chroma Technology Corp) inserted in the reticle space within the microscope eyepieces. In the dual-camera mode, invisible light sources were restricted to wavelengths below 420 nm or above 700 nm so that only the visible broadband light from the standard tungsten microscope lamp could reach the eye. Alternatively, the oculars could have been replaced with a lens tube that limits viewing to the video images on the computer monitor (Olympus part U-TLU).

The dual-camera microscope system could have also been employed as a single-monochrome camera multispectral imaging system described in Example 2. For multispectral imaging of both visible and invisible light channels, the dichroic beamsplitter in the dual-camera mount was replaced with a 100% reflective mirror to direct all light to the monochrome camera, and monochrome camera filters (E) were removed. Images of light transmitted for each light channel were acquired sequentially at the monochrome camera and image processing used for quantitative analysis, creation of color composite images, and performing spectral unmixing as described in Example 2. The specimen is not viewed through the oculars in the multispectral mode, but inadvertent viewing is protected by the visible-only transmitting filters in the eyepieces or replacement of oculars with a tube lens only part (Olympus U-TLU).

Similar illumination and imaging results can be obtained using other light sources and filtering. For example, a single light source emitting both visible light for illuminating the H&E and invisible light for illuminating the one or more detectable moieties can be employed, such as a tungsten halogen microscope lamp (IR filters removed), xenon lamp, or metal halide lamp, in combination with appropriate filtering. White light can be generated by a combination of LEDs, instead of a continuous light source, and combined with invisible LEDs to illuminate simultaneously both conventional stains and invisible chromogens.

Illumination channels for multispectral imaging (MS) and dual-camera real-time imaging (DC)

| | LED | bandpass filter, | | | |
|---|---|---|---|---|---|
| | nominal λ, | center λ/FWHM, | | dye | |
| | nm | nm | supplier | specificity | Imaging mode |
| 100 W | | 10HMR-0 hot | Olympus | conventional | DC |
| tungsten | | mirror, transmits | Newport Corp. | stain | DC/MS |
| halogen lamp | | 420-690nm | Chroma | HCC, DCC | MS |
| | | 405/30 | Technology | eosin | MS |
| | | 510/15 | Semrock | hematoxylin | DC/MS |
| | | 599/13 | Chroma | Cy7 | DC/MS |
| | | 769/49 | Technology | ir870 | |
| | | 880/40 | Semrock | | |
| | | | Chroma | | |
| | | | Technology | | |
| pE-4000 | 405 | — | CoolLED | HCC, DCC | DC/MS |
| light engine | 770 | | | Cy7 | DC/MS |
| Spectra X | 390 | 390/22 | Lumencor | HCC, DCC | DC/MS |
| light engine | 513 | 513/22 | Semrock | eosin | MS |
| | 620 | 620/19 | Semrock | hematoxylin | MS |
| | | | Semrock | | |

### Example 4. On-Slide Spectroscopic Measurements

Absorbance spectra of deposited chromogens and conventional stains were recorded on slide-mounted specimens placed on the stage of an Olympus BX-63 microscope under tungsten illumination. Transmitted light was measured between 350 and 800 nm in approximately 0.5 nm increments using a Pryor Scientific Inc. (Rockland, MA) Lumaspec 800 power meter. The power meter was upgraded with an Ocean HDX UV to NIR spectrometer that permitted spectral measurements between 200 and 1100 nm. The spectrum of light transmitted through a stained region of the slide was divided by the spectrum transmitted through on an unstained region to provide the transmission (T) spectrum, which was converted to the chromogen absorbance (A) spectrum using the relationship A=log 10(1/T).

Several detectable moieties were used individually in IHC targeting Ki67 on tonsil tissue and absorbance spectra of the resulting stained tissue are plotted in FIG. 10. Also plotted was the H&E absorbance spectrum on tonsil tissue and the visible range (approximated as 420 nm to 700 nm for a comfortable level of visible light illumination under the microscope) was tinted light blue. While portions of the indicated detectable moieties were within the visible region, the majority of their absorbance was outside the visible, and visual sensitivity was still low over most of the detectable moiety absorbance range. In addition, the H&E and detectable moiety spectra were normalized to the same peak value in FIG. 10, while in practice, the H&E absorbance was considerably greater than the detectable moieties and consequently the detectable moiety coloration is negligibly discernible when viewed under the microscope.

### Example 5. Pancreatic Formalin Fixed Paraffin Embedded (FFPE) Tissue Stained with Hematoxylin and Eosin (H&E) and Synaptophysin Invisible IHC (iIHC)

Formalin fixed paraffin embedded (FFPE) slide-mounted sections from normal (relative to cancer) pancreas tissue from an anonymized patient were prepared by Ventana Medical Systems, Inc. (VMSI) histology personnel from blocks obtained from the VMSI specimen bank. Semi-automated immunohistochemistry was performed on a DISCOVERY Ultra system (VMSI, Tucson AZ). The DISCOVERY Universal Procedure was used to create the protocol for controlling the staining steps. The procedure automated all steps utilizing commercial stainer reagents and paused the stainer for manual addition of custom chromogen reagents, as indicated in the procedure below. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-Synaptophysin primary antibody (rabbit; cat no. 790-4407), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 400 μM tyramide-modified DBCO in borate buffer pH 8.5 was added by manual pipetting, incubated for 4 min, followed by addition of 0.1 mL of 0.01% H₂O₂ in borate buffer pH 8.5. This was incubated for 32 min, followed by washing, and manual pipetting of 0.1 mL of 400 μM azide-modified Cy7, and incubation for 32 min. After washing, slides were removed from the stainer and washed in several hundred mL Reaction Buffer. Slides were then immediately stained with H&E or first dehydrated through ethanol and xylene (2×80% ethanol, 1 min each, 2×90% ethanol, 1 min each, 3×100% ethanol, 1 min each, 3× xylene, 1 min each), at ambient temperature. If dehydrated, slides were first re-hydrated by soaking in 100% ethanol for 1 min, 90% ethanol for 1 min, 80% ethanol for 1 min, and water for 1 min, at ambient temperature followed by H&E staining. H&E staining was performed in a series of Coplin jars at ambient temperature with manual transfer of slides between jars. Slides were first soaked in hematoxylin solution (Ventana HE 600 Hematoxylin; order code 07024282001) for 2 min followed by water for 2 min, define solution (Leica Surgipath SelectTech Define MX-aq, cat. no. 3803595) for 1 min, water for 1 min, bluing solution (VWR Bluing reagent; cat. no. 95057-852) for 1 min, water for 1 min, 95% ethanol for 30 s, eosin solution (Ventana HE 600 Eosin; order code 06544304001) for 1 min, 70% ethanol for 1 min, 100% ethanol twice for 1 min each, and xylene three times for 1 min each. Slides were then allowed to dry and were mounted by applying Richard Allan Scientific Cytoseal XYL (ThermoFisher Scientifc, Kalamazoo, MI) and covering with a type 1.5 coverslip, or were mounted on a Sakura FineteK USA (Torrance, CA) Tissue-Tek Film Automated Coverslipper.

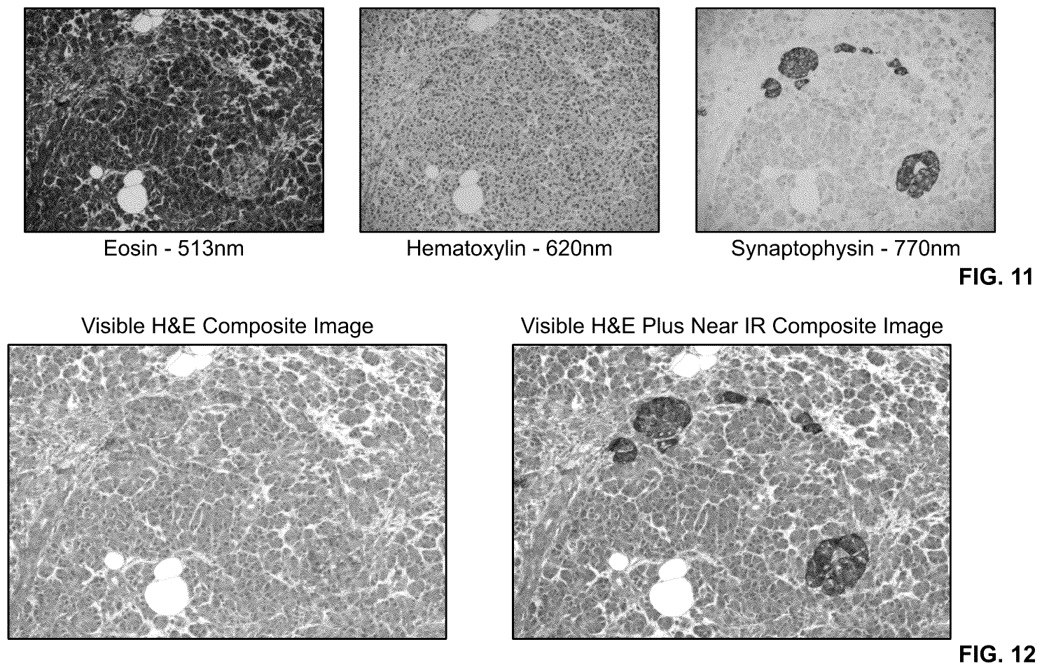

To test the ability to combine invisible IHC with H&E staining, IHC targeting the protein synaptophysin was performed on FFPE normal pancreatic tissue followed by conventional H&E staining. The IHC utilized a detectable moiety comprising the dye C7 that absorbs light in the far red/near IR region of the spectrum (see FIG. 10). Visual examination through the ocular of the microscope under white light illumination (tungsten halogen lamp) revealed normal H&E staining of the specimen. FIG. 11 provides images of transmitted light recorded with a monochrome camera using LED illumination at 513 nm where eosin absorbs light, 620 nm where hematoxylin absorbs light, and 770 nm where Cy7 absorbs light. These three images were combined into color composite images as presented in FIG. 12. The color image on the left results from the combination of the 513 nm and 620 nm images with pseudo-coloring that faithfully reproduces visualization under the microscope with white light illumination. The color image on the right combined the 770 nm image, pseudo-colored black, with the 513 and 620 nm images and shows the presence of synaptophysin within the context of the pancreatic tissue as revealed by the H&E.

### Example 6. FFPE Tonsil Tissue (Normal with Respect to Cancer) Stained with H&E and CD20+CD8 Multiplex iIHC

Formalin fixed paraffin embedded (FFPE) slide-mounted sections from normal (relative to cancer) tonsil tissue from an anonymized patient were prepared by Ventana Medical Systems, Inc. (VMSI) histology personnel from blocks obtained from the VMSI specimen bank. Semi-automated immunohistochemistry was performed on a DISCOVERY Ultra system (VMSI, Tucson AZ). The DISCOVERY Universal Procedure was used to create the protocol for controlling the staining steps. The procedure automated all steps utilizing commercial stainer reagents and paused the stainer for manual addition of custom chromogen reagents, as indicated in the procedure below. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-CD20 primary antibody (mouse; VMSI cat no. 760-2531), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 600 μM tyramide-modified DBCO in borate buffer pH 8.5 was added by manual pipetting, incubated for 4 min, followed by addition of 0.1 mL of 0.01% H2O2 in borate buffer pH 8.5. This was incubated for 32 min, followed by washing, and manual pipetting of 0.1 mL of 600 μM azide-modified DCC and incubation for 32 min. After washing, the slides were incubated with Cell Conditioning 2 (VMSI Cat #950-123) at 100° C. for 8 min., followed by washing in reaction buffer. Anti-CD8 primary antibody (rabbit; VMSI cat no. 790-4460), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 400 μM tyramide-modified DBCO, was added by manual pipetting, and incubated for 4 min. 0.1 mL of 0.01% H2O2 in borate buffer pH 8.5 was added and incubated for 32 min, followed by washing, and manual pipetting of 0.1 mL of 400 μM azide-modified Cy7 onto the slide with incubation for 32 min. After washing, slides were removed from the stainer and washed in several hundred mL Reaction Buffer Slides were then stained with H&E, mounted, and coverslipped as described in Example 5.

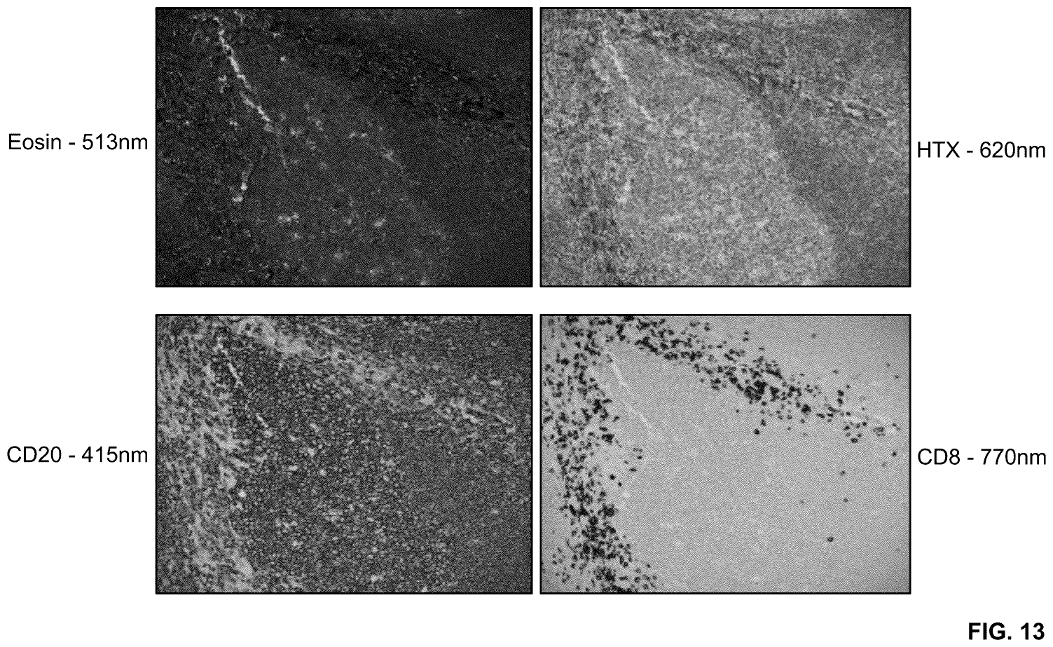

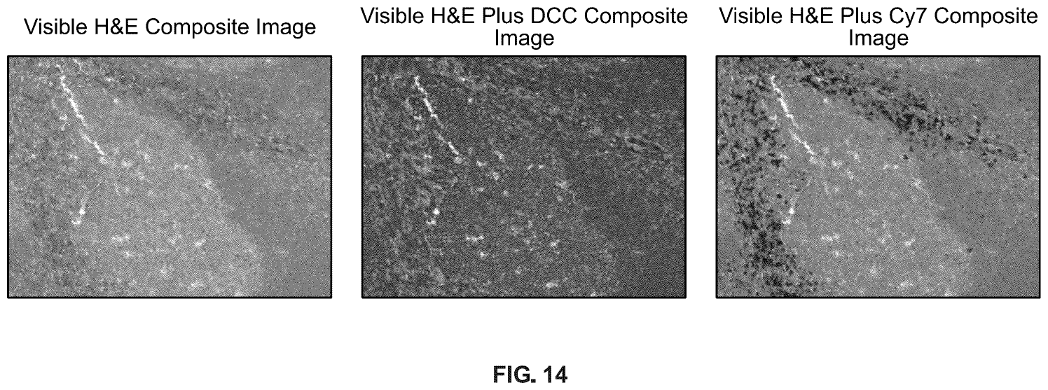

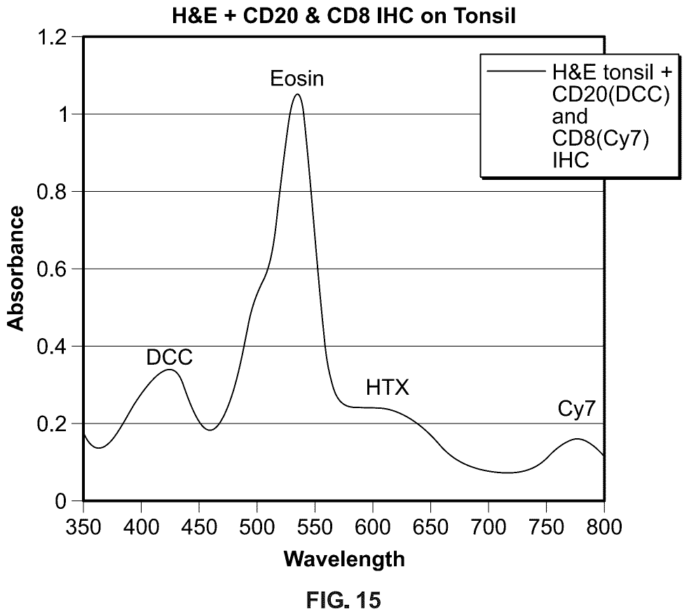

Multiplex invisible IHC with H&E staining was demonstrated by performing sequential IHC targeting CD20 and CD8 on FFPE tonsil tissue (normal with respect to cancer) followed by H&E staining. CD20 was stained with a detectable moiety derived from diethylamiocoumarincarboxylate (DCC), a far blue absorbing dye, and CD8 was stained with the Cy7 detectable moiety. Absorbing in the far blue near the edge of visual perception, DCC can appear weakly yellow by eye with heavy staining but is barely perceptible in the presence of the H&E stain. FIG. 13 displays the four monochrome images recorded with the 513, 620, and 770 nm LEDs from the pancreas example, and a 415 nm LED where the DCC absorbs light. The characteristic B-cell membrane staining of CD20 throughout the tonsil as was evident in the 415 nm image and the characteristic activated t-cell membrane staining of CD8 largely outside the geminal center as evident in the 770 nm image. Color composite images are depicted in FIG. 14 with the H&E image on the left constructed from the 513 and 620 nm images faithfully reproducing the visual observation. The center image shows the addition of the 415 nm (CD20) image to the H&E composite and the right image shows the addition of the 770 nm (CD8) image to the H&E composite, both IHC targets pseudo-colored black to set them apart from the H&E staining. FIG. 15 illustrates an absorbance spectrum of this tissue in which the two detectable moiety absorbance peaks are clearly separated from the eosin and hematoxylin absorbance.

### Example 7. FFPE Colon Tumor Tissue Stained with H&E and CD3+CD8 Multiplex iIHC

Formalin fixed paraffin embedded (FFPE) slide-mounted sections from colon tumor tissue from an anonymized patient were prepared by Ventana Medical Systems, Inc. (VMSI) histology personnel from blocks obtained from the VMSI specimen bank. Semi-automated immunohistochemistry was performed on a DISCOVERY Ultra system (VMSI, Tucson AZ). The DISCOVERY Universal Procedure was used to create the protocol for controlling the staining steps. The procedure automated all steps utilizing commercial stainer reagents and paused the stainer for manual addition of custom chromogen reagents, as indicated in the procedure below. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-CD3 primary antibody (VMSI cat no. 790-4341), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 400 μM tyramide-modified DBCO in borate buffer pH 8.5 was added by manual pipetting, incubated for 4 min, followed by addition of 0.1 mL of 0.01% H2O2 in borate buffer pH 8.5. This was incubated for 32 min, followed by washing, and manual pipetting of 0.1 mL of 400 μM azide-modified HCCA and incubation for 32 min. After washing, the slides were incubated with Cell Conditioning 2 (VMSI Cat #950-123) at 100° C. for 8 min., followed by washing in reaction buffer. Anti-CD8 primary antibody (cat no. 790-4460), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 200 μM tyramide-modified DBCO, was added by manual pipetting, and incubated for 4 min. 0.1 mL of 0.01% H2O2 in borate buffer pH 8.5 was added and incubated for 32 min, followed by washing, and manual pipetting of 0.1 mL of 200 μM azide-modified Cy7 onto the slide with incubation for 32 min. After washing, slides were removed from the stainer and washed in several hundred mL Reaction Buffer Slides were then stained with H&E, mounted, and mounted/coverslipped as described in Example 5.

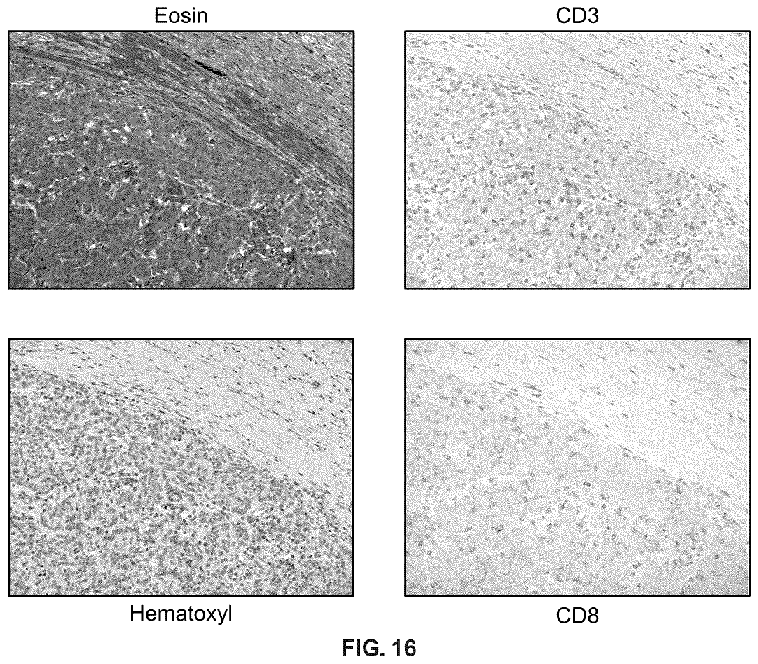

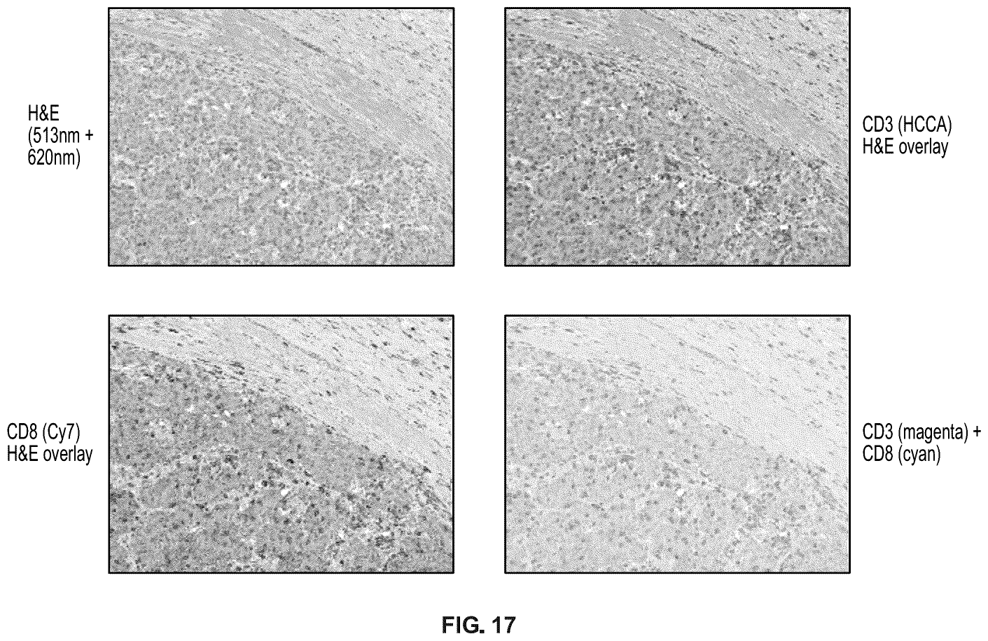

Multiplex IHC with H&E staining was applied to FFPE colon tumor tissue, targeting the general t-cell marker CD3, and the marker of t-cell activation CD8. CD3 was stained with the HCCA detectable moiety and CD8 was stained with the Cy7 detectable moiety. FIG. 16 presents the monochrome transmitted light images recorded using four LED light channels, 390 nm, 770 nm, 513 nm, and 620 nm, that emphasize the HCCA and Cy7, and eosin and hematoxylin absorbance, respectively, clearly identifying the t-cell populations. FIG. 16 presents color composite images constructed from combinations of the four images. The color composite in the top left of FIG. 17 reproduced the visual H&E staining pattern and the top right and lower left images add the invisible CD3 and CD8 staining, respectively, pseudo-colored as black. In the CD3 and CD8 composite images, the H&E staining has been reduced to better visualize the biomarkers. The lower right image combines only the CD8 image, pseudo-colored cyan, and CD3 image, pseudo-colored magenta. Activated t-cells, which co-expressed CD8 with CD3, appear blue (magenta+cyan=blue), while non-activated t-cells appear magenta.

This colon tumor example demonstrated an important advantage of performing H&E and IHC simultaneously on a single slide. It has been demonstrated in colon carcinoma that the distribution of CD3 and CD8 cells in the core tumor and invasive margin is a strong prognostic marker in colorectal cancer for disease-free and overall survival (see Galon J, Mlecnik B, Bindea G, Angell HK, Berger A, Lagorce, A L, et al. Towards the introduction of the ‘immunoscore’ in the classification of malignant tumors. J Pathol 2014; 232:199-209). Typically, three FFPE sections are required for the analysis—one for H&E, and one each for CD3 and CD8. The location of the invasive margin was identified on the H&E slide and then transferred to the CD3-stained and CD8− stained slides on which the respective cell densities relative to the margin were measured. However, since the CD3 and CD8 cells were enumerated on sequential (serial) FFPE sections, the tumor periphery as somewhat altered on each slide, due to different orientations the three sections when applied to the slides, and because each section cut through a deeper portion of the tumor and the size, shape, and orientation would change with the depth and number of cuts. This required the tumor margin measured on the H&E slide be adjusted for any orientation and tumor alteration that occurred on the CD3 and CD8 slides, based on tumor morphology identified using each single IHC stain and a hematoxylin nuclear stain. While this approximation of the tumor margin on the IHC slides has been shown to provide clinically significant results, performing H&E and dual IHC on the same slide removes the uncertainty of transferring the margin location and, therefore, should provide a more accurate result. This would presumably increase the association between CD3/CD8 cell densities relative to tumor margins with outcome and further improve the prognostic strength of the assay.

### Example 8. FFPE Breast Tumor Xenograft Tissue Stained with H&E and HER2 iIHC, and Evaluated Using Dual-Camera Color/Monochrome Imaging System

Formalin fixed paraffin embedded (FFPE) slide-mounted sections from breast tumor xenografts (VMSI Ventana HER2 Dual ISH 3-in-1 Xenograft Slides, REF 783-4422) were stained by IHC using semi-automated immunohistochemistry on a DISCOVERY Ultra system (VMSI, Tucson AZ) similar to Example 5. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-HER2/neu primary antibody (VMSI cat no. 790-2991), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 300 μM tyramide-modified DBCO in borate buffer pH 8.5 was added by manual pipetting, incubated for 4 min, followed by addition of 0.1 mL of 0.01% H2O2 in borate buffer pH 8.5. This was incubated for 32 min, followed by washing, and manual pipetting of 0.1 mL of 300 μM azide-modified Cy7, and incubation for 32 min. After washing, slides were removed from the stainer and washed in several hundred mL Reaction Buffer. H&E staining and mounting/coverslipping were performed as described in Example 5.

In examples 5, 6, and 7, hematoxylin, eosin, and the invisible chromogens were illuminated sequentially with bands of light near the peak absorbance of each dye or chromogen, each imaged with a monochrome camera, and the resulting images viewed separately or combined into a composite image (see Morrison L E, Lefever M R, Behman L J, Leibold T, Roberts E A, Horchner U B, Bauer D R. Brightfield Multiplex Immunohistochemistry with Multispectral Imaging. Lab Invest 2020). Another approach would be to illuminate simultaneously with white light and invisible light and split the transmitted light between a color camera and a monochrome camera. With proper filtering the H&E image can be viewed on the color camera simultaneously with the detectable moiety image on the monochrome camera, presented side-by-side and/or overlaid as a single image on a computer monitor. Both images can be presented at video rates such that the microscope stage position, focus, and overlay image (relative contribution of the two images) can be changed while viewing the live images. If desired, the operator could also view through the microscope ocular.

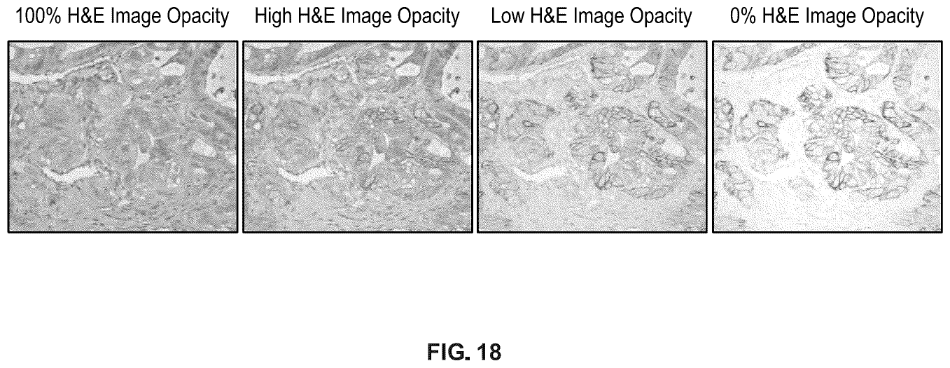

HER2 IHC was performed on a breast tumor xenograft using the Cy7 detectable moiety followed by H&E staining, and images recorded with the 2-camera system are shown in FIG. 18. The H&E image recorded with the color camera was displayed on the far left of the figure and the monochrome image of the Cy7 detectable moiety staining is displayed on the far right. Between are two overlay images with greater and lesser contribution (opacity) of the H&E image relative to the Cy7 (HER2) image. The value of the split image approach was such that the pathologist can interactively view both the H&E and IHC staining patterns in real time, traversing the entire slide as in a conventional slide evaluation. Areas of diagnostic interest could be documented by stopping and imaging individual fields with both cameras, as desired. As with the sequential image approach using composite images in earlier examples, H&E and IHC staining could be correlated by visual evaluation of the overlays down to the single cell level, providing a distinct advantage over separate H&E and IHC staining on serial tissue sections or multiple cytology slides.

The split image approach in FIG. 18 allowed simultaneous viewing of the H&E staining pattern and one biomarker. When multiplex IHC is employed, the split image approach could display the H&E image continuously next to each biomarker image sequentially by changing the invisible illumination channel to light match the absorbance of each detectable moiety. For simultaneous viewing of H&E and multiple biomarkers, splitting of microscope transmitted light could be extended beyond two-fold splitting. Like the commercial Thorlabs 2-camera splitter, used in the HER2/H&E example (FIG. 18), Cairn Research (Kent, UK; also distributed by Teledyne Photometrics, Tucson, AZ) has commercialized 2- and 4-camera splitters, as well as splitters that deliver up to four different offset images of the same microscope field, individually filtered, to a single camera sensor. This latter splitter displays each of the four split images in a separate quadrant of the resulting single camera image. Both the 4-camera splitter and single camera with offset images would permit simultaneous viewing of the H&E staining and detectable moiety staining of up to three multiplexed biomarkers.

### Example 9. Cervical Cytology Specimen Stained with PAP Conventional Stain and Ki-67+p16 Multiplex Invisible Immunocytochemistry (iICC)

A specimen of cervical cells pooled from multiples anonymized patients was applied to microscope slides using the ThinPrep (Hologic, Mississauga, ON) procedure. ICC was performed on the preparation according to the VMSI CINtec PLUS Cytology protocol (package insert). CINtec PLUS Cytology detection was performed on a BenchMark Ultra system using the CINtec PLUS Cytology cocktailed (p16/Ki-67) primary antibodies and detection reagents. In general, ICC was performed at 36° C., unless otherwise noted, and Reaction Buffer wash solutions were diluted from 10× concentrate. Antigen retrieval for cervical specimen pools was performed by applying Cell Conditioning 1 and warming the slide to 75° C. for 4 min, then increasing the temperature to 100° C. for 24 minutes. Since the CINtec PLUS Cytology antibodies are cocktailed together, incubation with both primary antibodies was performed concurrently, followed by washing in Reaction Buffer to remove unbound antibody. Incubation with anti-species antibodies was performed sequentially targeting the primary antibody (either anti-mouse or anti-rabbit) conjugated to peroxidase. DCC and Cy7 iCDCs were substituted for the conventional DAB and Fast Red chromogens, using tyramide DBCO and azide-modified DCC and Cy7 as described above for multiplex IHC. At the conclusion of the staining run, the slides were washed with a dilute detergent solution (1 drop of Dawn dish liquid (Proctor & Gamble, Cincinnati, OH) in 200 mL water). Conventional PAP staining was performed immediately while slides were still wet, as follows. The damp slides were soaked in distilled water for 1 minute, Richard-Allan Hematoxylin I (ThermoFisher) for 30 seconds, distilled water twice for 15 seconds each, Richard-Allan Clarifier 1 for 30 seconds, distilled water for 30 seconds, Richard-Allan Bluing Reagent for 30 seconds, 50% ethanol for 30 seconds, 95% ethanol for 30 seconds, Richard-Allan Scientific™ Cyto-Stain™ (ThermoFisher) for 1 minute, twice in 95% ethanol for 30 seconds each, followed by three passes through three clean baths of 100% ethanol for 30 seconds each, three passes through xylene, two for 1 minute each and the last for 3 minutes. The PAP-stained slides were then coverslipped as described in Example 5 for H&E staining.

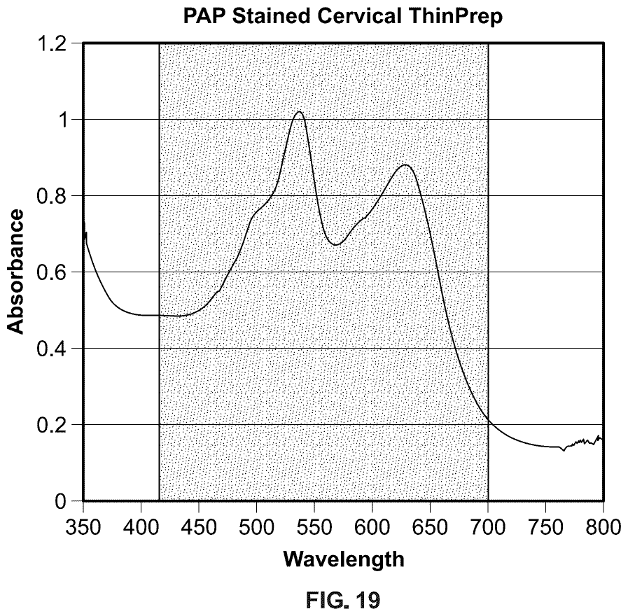

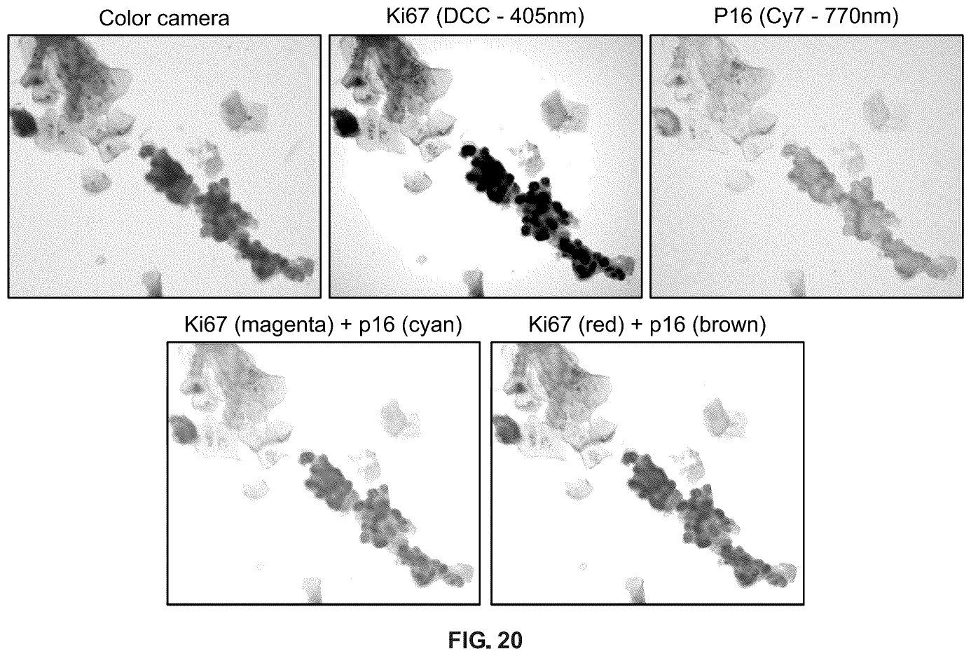

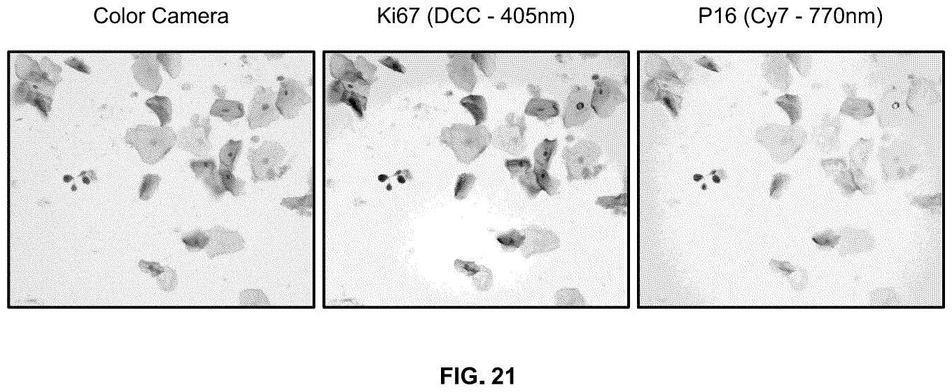

In this example, the H&E plus invisible chromogen concept was extended to another conventional pathology stain and another preparation type by combining multiplex iICC with PAP staining of cervical cytology specimens targeting the tumor suppressor p16 and a marker for cell proliferation Ki67. FIG. 19 shows the absorbance spectrum of a PAP-stained cervical cytology specimen, indicating that similar to H&E, PAP stain absorbed strongly in the visible spectrum, less in the deep blue/UV, and minimally absorbs in the far-red/near-IR. FIG. 20 displays images of a collection of cervical cells, with the top left image recorded on the color camera in the 2-camera system. A cluster of cells in the lower portion of the image appeared abnormal, and this was confirmed by p16 staining with the DCC chromogen (405 nm LED) and Ki67 staining with the Cy7 chromogen (770 nm LED) shown in the upper middle and right monochrome images, respectively, and recorded with the monochrome camera. The lower two images combined the DCC and Cy 7 images with different pseudo-coloring to showed better the simultaneous staining. The lower right image represented Ki-67 in red and p16 in brown to imitate the CINtech commercial assay for expression of these two proteins which was performed in the absence of PAP staining. The lower left image represented Ki-67 as magenta and p16 as cyan to improve definition. FIG. 21 shows another collection of cervical cells with PAP staining in the left image, recorded with the color camera, and Ki67 (DCC) and p16 (Cy7) staining in the middle and right images, recorded on the monochrome camera. The grouping of 4 cells to the left of center in the three images clearly showed abnormality with strong staining for both biomarkers.

The cervical cytology examples demonstrated that a common conventional stain other than H&E could be used to advantage with invisible IHC, and that cytology preparations are also suitable specimens. PAP stain is used to find abnormal cervical cells in brushing specimens to identify women on a path to cervical cancer. The specimens are dispersed cervical cells and cell aggregates applied to slides as smears or by commercial liquid cytology methods such as ThinPrep (Hologic, Mississauga, ON) and Surepath (Becton Dickinson and Company, NJ). Historically, cell morphology of cervical cells, as revealed by PAP staining, served as the primary method for screening patients for cervical cancer and dysplasia potentially leading to cervical cancer. More recently, cervical cells overexpressing both p16 and Ki-67, identified by IHC, have been shown to add additional benefit in identifying abnormal cells (see Wright Jr T C, et al. Triaging HPV-Positive Women with p16/Ki-67 Dual-stained Cytology: Results from a Sub-study Nested into the ATHENA Trial. Gynecol Oncol 2017; 144:51-56.Gynecol Oncol). The ability to evaluate the PAP staining pattern, p16 expression, and Ki-67 expression in every cell by combining PAP and dual IHC as demonstrated here, might well be expected to provide even greater diagnostic accuracy.

### Example 10. Use of Special Stains with iIHC

The following special stains were performed on the Ventana Special Stains Automated Slide Stainer using Ventana special stain kits, following the manufacturer's suggested protocols: Trichrome Green (order code 06521916001), Trichrome Blue (order code 06521908001), Jones Light Green (order code 05279356001),), Jones H&E (order code 05279348001), and Acid Fast Bacteria (AFB; order code 08432503001).

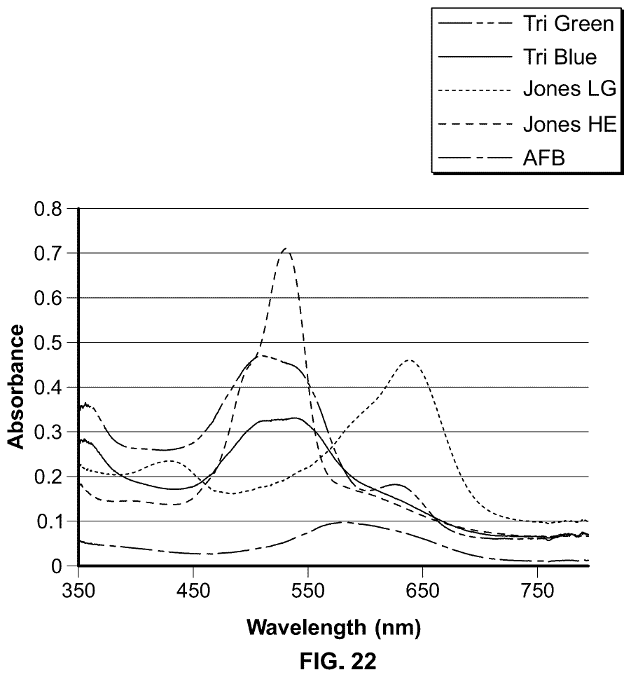

A number conventional histology and cellular stains other than H&E and PAP are used routinely in anatomical pathology to aid in the identification of abnormality and disease, and are referred to commonly as special stains. The absorbance spectra of several special stains applied to slide-mounted cellular specimens are plotted in FIG. 22 and show that although they have strong absorbance within the visible spectrum, the absorbance is greatly reduced in either the deep-blue/UV or far-red/near-IR spectral regions, or both. Therefore, as with H&E and PAP stains, many special stains also permit the co-application of iIHC and iICC.

### Example 11. Melanoma FFPE Tissue Stained with H&E and MART-1/Melan A iIHC

Melanoma FFPE tissue was stained by IHC using semi-automated immunohistochemistry on a DISCOVERY Ultra system (VMSI, Tucson AZ) similar to Example 5. Each step was performed at 37° C., except as noted, with mixing, and automated wash steps used Reaction Buffer (diluted from 10× concentrate; VMSI cat. no. 950-300). Slide-mounted paraffin sections were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-MART1/melanA primary antibody (VMSI cat no. 790-2990), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 400 μM tyramide-modified DBCO in borate buffer pH 8.5 was added by manual pipetting, incubated for 4 min, followed by addition of 0.1 mL of 0.01% H2O2 in borate buffer pH 8.5. This was incubated for 32 min, followed by washing, and manual pipetting of 0.1 mL of 400 μM azide-modified Cy7, and incubation for 32 min. After washing, slides were removed from the stainer and washed in several hundred mL Reaction Buffer. H&E staining and mounting/coverslipping were performed as described in Example 5.

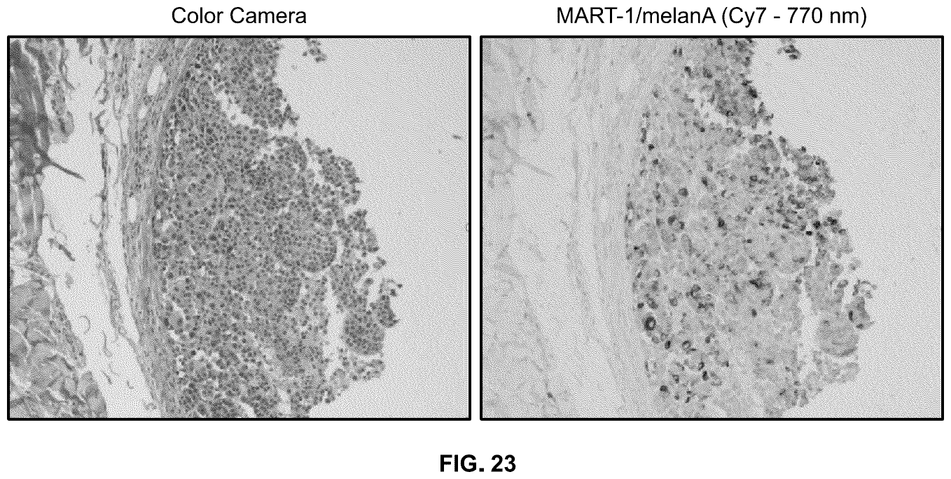

Another application of detectable moieties is for IHC on tissues containing endogenous or exogenous pigments that absorb strongly in the visible spectrum and interfere with visualization or imaging of the IHC staining. An example of an endogenous pigment is melanin, a polymer found in skin melanocytes that protects the body from harmful UV irradiation. The absorbance spectrum of melanin in skin, as determined by remission spectroscopy, shows strong absorbance in the UV that decreases through the visible portion of the spectrum to the point that it is greatly reduced in the far red and near-IR.17 Images of melanoma FFPE tissue stained both with H&E and IHC targeting MART-1/melan A stained with Cy7 detectable moiety, are displayed in FIG. 23. The color image, recorded with the 2-camera system, revealed the presence of brown melanin pigment within the H&E staining, whereas the monochrome image showed absorbance of the 770 nm LED light by the Cy7 detectable moiety that clearly defines the MART1/melanA, with essentially no melanin interference. In tissues with heavy melanin deposits, the melanin absorbance and/or light scattering was apparent at 770 nm but markedly attenuated.

The melanin example demonstrated that invisible IHC was not only beneficial for application with applied stains such as H&E, PAP, and special stains, but was also valuable for use in combination with strongly absorbing endogenous pigments, or even exogenous pigments due to industrial or environmental exposure through inhalation, ingestion, or contact, including tattoo pigments. In specimens containing melanin, interpretation of IHC with conventional chromogens can be difficult. Chemical bleaching of the melanin was often employed to reduce the melanin absorbance to a level that permits evaluation of the IHC. However, the bleaching can sometimes alter the targeted antigens, reducing the efficiency of their detection, and could also bleach the IHC chromogens. It is believed that the use of the invisible chromogens (such as described herein) allows the bleach to be avoided completely.

### Example 11. Tonsil FFPE Tissue Stained by Kappa mRNA In Situ Hybridization (ISH) and H&E

mRNA ISH was performed as previously described substituting 100 μL of 50 or 100 μM Cy7 detectable moiety in TSA diluent. H&E staining and mounting/coverslipping were performed as described in Example 5.

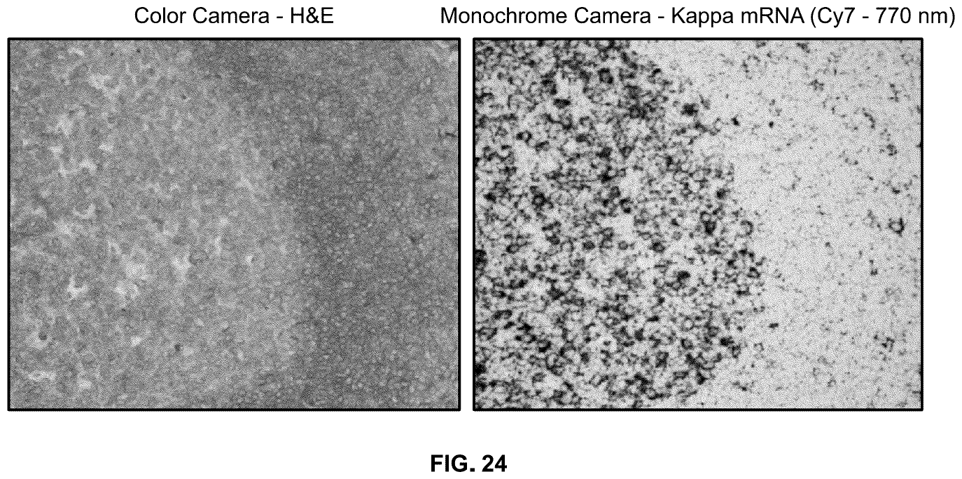

H&E combined with invisible in situ hybridization (ISH) was also demonstrated, as shown in FIG. 24 on tonsil FFPE tissue. The left image was recorded with the color camera in the 2-camera system and the right image is recorded with the monochrome camera viewing the 770 nm LED light absorbed by the Cy7 detectable moiety. Instead of the primary antibody reagents used in IHC, haptenated nucleic acid probes hybridized to the kappa mRNA sequences direct enzymatic covalent deposition and staining with Cy7 detectable moiety via enzyme-conjugated anti-hapten antibodies. As expected, the kappa expression in tonsil varied from very strongly expressing cells to very weakly expressing cells, seen as only one to several spots per weakly expressing cell. ISH is often criticized for its lack of tissue and cellular morphology, however, this example proves that ISH can presented within the context of H&E stain, albeit with some degradation of the H&E quality due to more stringent specimen pre-treatment, including proteolysis, to improve access of nucleic acid targets to probes.

### Example 12. Chromogen Deposition Chemistry

Formalin fixed paraffin embedded (FFPE) slide-mounted sections from normal (relative to cancer) tonsil tissue from an anonymized patient were prepared by Ventana Medical Systems, Inc. (VMSI) histology personnel from blocks obtained from the VMSI specimen bank. Specimen slides were stained by IHC using semi-automated immunohistochemistry on a DISCOVERY Ultra system (VMSI, Tucson AZ) similar to Example 5 except for the chromogen deposition as described below. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-CD8 primary antibody (cat no. 790-4460), 0.1 mL, was added and incubated for 16 to 32 min before washing to remove unbound antibody. OmniMap anti-Rb HRP (RUO), DISCOVERY (VMSI Cat #760-4311), for tyramide chromogens, or UltraMap anti-Rb Alk Phos, DISCOVERY (VMSI Cat #760-4314), for quinone methide precursor chromogens, 0.1 mL, was added and incubated for 8 min followed by washing. 0.1 mL of 1200 tyramide derivative of 7-amino-4-methylcoumarin-3-acetate (AMCA-tyramide) in borate buffer pH 8.5 or 400 uM quinone methide precursor modified Cy7 in borate buffer at a pH of 8.5 was added by manual pipetting. Tyramide chromogens were incubated for 4 min followed by addition of 0.1 mL of 0.01% H2O2 in borate buffer pH 8.5, and incubated for 32 min, followed by washing. Quinone methide precursor modified chromogens were incubated for 32 min, followed by washing. Slides were then were removed from the stainer and washed in several hundred mL Reaction Buffer. H&E staining and mounting/coverslipping were performed as described in Example 5.

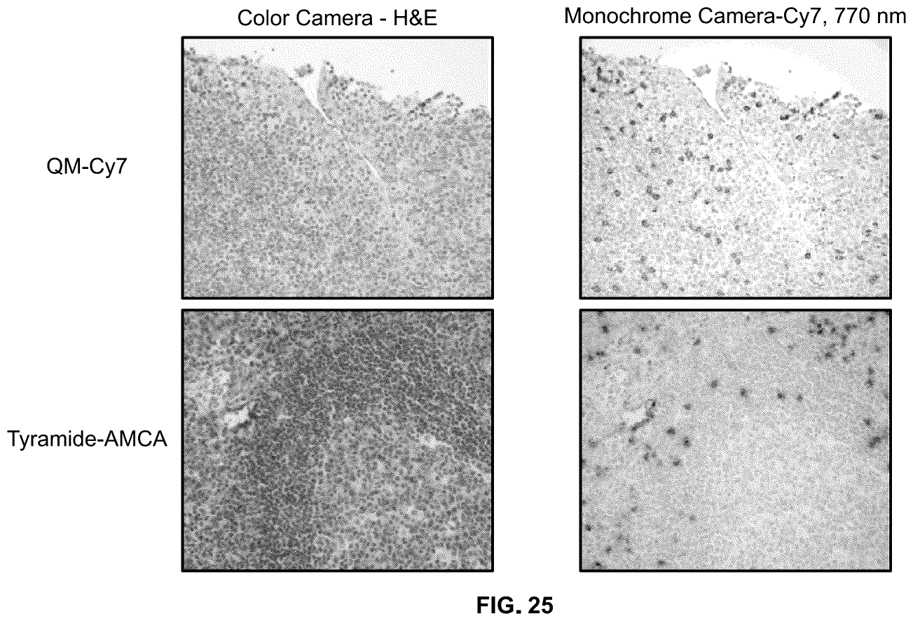

In previous examples, detectable moieties were covalently deposited via a ‘click chemistry’ intermediate, using peroxidase-catalyzed covalent deposition of a DBCO-tyramide derivative followed by coupling with azide-derivatized dye. Tyramide derivatives of dyes, deposited by peroxidase catalyzed free radical formation, and quinone methide precursor dye derivatives, deposited by alkaline phosphatase hydrolysis to form reactive quinone methides, were also effective for invisible staining of biomarkers. This was shown in FIG. 25 in which tonsil FFPE tissue is stained with CD8 invisible IHC using a quinone methide-Cy7 detectable moiety (top) and a tyramide-AMCA UV-absorbing detectable moiety (bottom). The 2-camera system was utilized with the color camera images shown on the left and the monochrome images shown on the right of the figure. This demonstrated that any chromogen with absorbance primarily in the invisible regions of the spectrum may be considered for use with conventional visible-absorbing stains such as H&E, regardless of the deposition chemistry. One caveat is that the chromogens must resist removal by the reagents and conditions utilized in the conventional staining when applied after the IHC. In this regard, the detectable conjugates (including detectable moieties) are particularly useful since they are covalently attached to cellular and tissue components and will not be solubilized by organic or aqueous reagents, as opposed conventional chromogens such as Fast Red.

### Example 13. Mucicarmine Special Stain Plus Invisible TTF-1 and p40 Dual IHC on Lung Tumor Tissue

FFPE slide-mounted sections from lung tumor tissue from an anonymized patient were prepared by Ventana Medical Systems, Inc. (VMSI) histology personnel from blocks obtained from the VMSI specimen bank. Semi-automated immunohistochemistry was performed on a DISCOVERY Ultra system (VMSI, Tucson AZ). The DISCOVERY Universal Procedure was used to create the protocol for controlling the staining steps. The procedure automated all steps utilizing commercial stainer reagents and paused the stainer for manual addition of custom chromogen reagents, as indicated in the procedure below. Each step was performed at 37° C., except as noted, with mixing, and automated wash steps used Reaction Buffer (diluted from 10× concentrate; VMSI cat. no. 950-300). Slide-mounted paraffin sections, up to 30 per run, were processed on the instrument beginning with de-paraffinization with warming of the slides to 70° C. for 3 cycles, each 8 min long. Antigen retrieval was performed by applying Cell Conditioning 1 (VMSI Cat. no. 950-124) and warming the slides to 94° C. for 64 min. Anti-Thyroid Transcription Factor-1 (TTF-1; SP141) rabbit monoclonal primary antibody (cat no. 790-4756), 0.1 mL, was added and incubated for 16 to 32 min before washing to remove unbound antibody. OmniMap anti-Rb HRP (RUO), DISCOVERY (VMSI Cat #760-4311), 0.1 mL was added and incubated for 8 min followed by washing. 0.1 mL of 100-400 μM tyramide-modified DBCO in borate buffer pH 8.5 was added by manual pipetting, incubated for 4 min, followed by addition of 0.1 mL of 0.01% H2O2 in borate buffer pH 8.5. This was incubated for 32 min, followed by washing, and manual pipetting of 0.1 mL of 100-400 μM azide-modified IR870 and incubation for 32 min. After washing, the slides were incubated with Cell Conditioning 2 (VMSI Cat #950-123) at 100° C. for 8 min., followed by washing in reaction buffer. Anti-p40 (BC28) mouse monoclonal primary antibody (VMSI cat no. 790-4950), 0.1 mL, was added and incubated for 16 to 32 min before washing to remove unbound antibody. OmniMap anti-Ms HRP (RUO), DISCOVERY (VMSI Cat #760-4310), 0.1 mL was added and incubated for 8 min followed by washing. 0.1 mL of 200-400 μM tyramide-modified DBCO, was added by manual pipetting, and incubated for 4 min. 0.1 mL of 0.01% H2O2 in borate buffer pH 8.5 was added and incubated for 32 min, followed by washing, and manual pipetting of 0.1 mL of 200-400 μM azide-modified Cy7 onto the slide with incubation for 32 min. After washing, slides were removed from the stainer and washed in one or more of the following: 250 mL Reaction Buffer Slides, 250 ml water containing 0.2 g Dawn dishwashing detergent, 250 ml water. Slides were then immediately stained with H&E or first dehydrated through ethanol and xylene (2×80% ethanol, 1 min each, 2×90% ethanol, 1 min each, 3×100% ethanol, 1 min each, 3× xylene, 1 min each), at ambient temperature. If dehydrated, slides were first re-hydrated by soaking in 100% ethanol for 1 min, 90% ethanol for 1 min, 80% ethanol for 1 min, and water for 1 min, at ambient temperature followed by mucicarmine staining. Mucicarmine staining was performed in a series of Coplin jars at ambient temperature with manual transfer of slides between jars using a mucicarmine staining kit (cat no. ab150677; Abcam, Cambridge, MA) following manufacturer instruction. This included soaking slides 3 min in hematoxylin solution, washing in water, soaking slides 30 s in bluing reagent, washing in water, soaking slides in Mucicarmine solution for 10 min, washing slides in water, soaking slides in tartrazine solution for 1 min, rinsing slides in several changes of ethanol, and soaking slides in xylene. Slides were then drained and mounted by applying Richard Allan Scientific Cytoseal XYL (ThermoFisher Scientifc, Kalamazoo, MI) and covering with a type 1.5 coverslip.

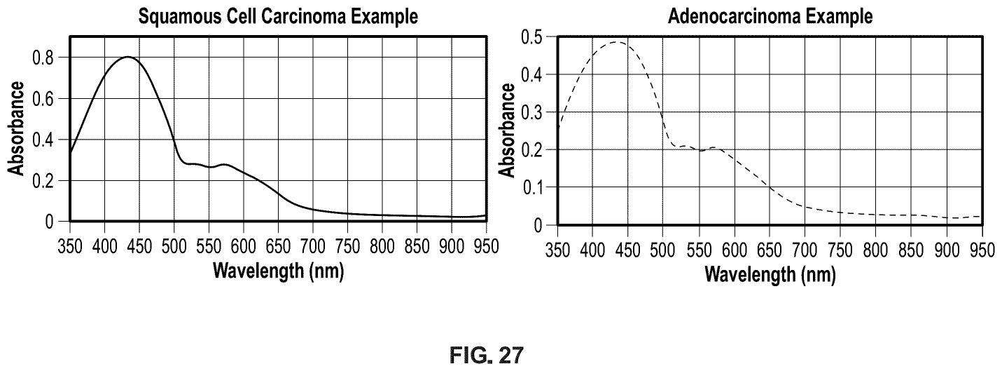

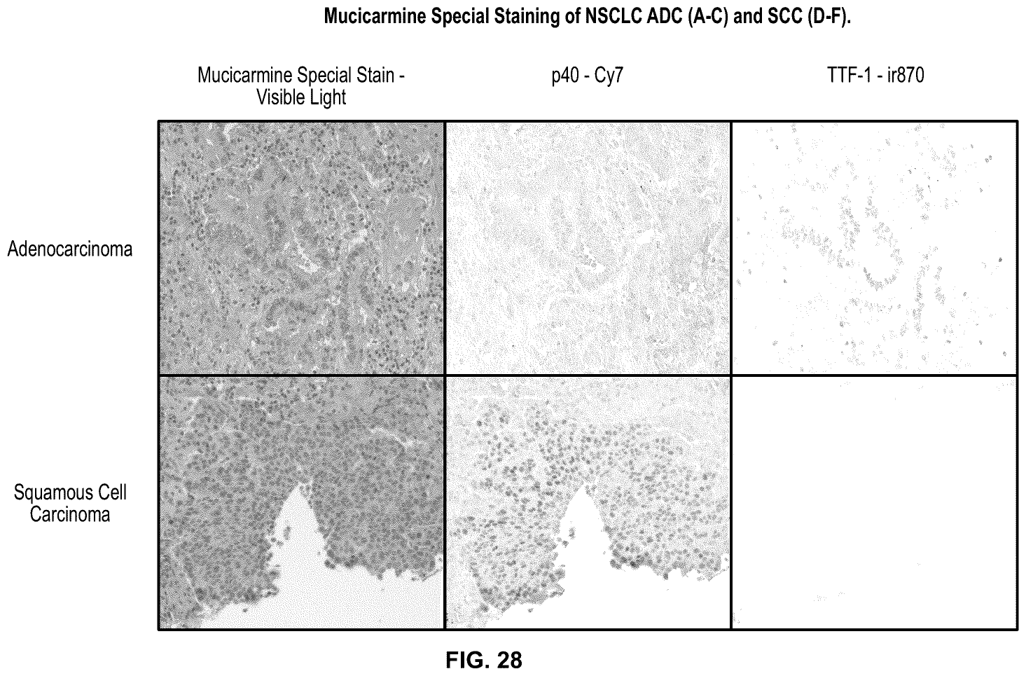

Mucicarmine special stain is often used by pathologists to aid in the classification of lung tumor tissue. De-paraffinized lung tumor tissues, one squamous cell carcinoma and one adenocarcinoma, were stained according to the above procedure (without IHC) and the absorbance spectra were recorded, and plotted in FIG. 27. IHC can provide even greater ability to distinguish squamous cell carcinoma and adenocarcinoma and would therefore be a good complement to mucicarmine special stain. TTF-1 IHC specifically stains cells in adenocarcinoma and p40 IHC specifically stains cells in squamous cell carcinoma. The spectra indicate that mucicarmine stain absorbance may interfere with the use of deep-blue- and UV-absorbing HC chromogens but should not interfere with the use of far-red/near-IR absorbing chromogens. To demonstrate the use mucicarmine special stain combined with IHC, squamous cell carcinoma and adenocarcinoma lung tumor tissues were stained by dual iIHC to identity p40 and TTF-1 expressing cells, using the Cy7 (absorbance maximum 774 nm) and IR870 (absorbance maximum 869 nm) chromogens, respectively, followed by mucicarmine staining as described in the above procedure. Images of each stained tissue were recorded with the dual-camera microscope system, using a tungsten lamp source with a Semrock 769 nm center wavelength, 49.3 nm FWHM bandpass filter (cat no. FF01-769/41; IDEX Health & Science, LLC, Rochester, NY) and a Chroma 880 nm center wavelength, 40 nm FWHM bandpass filter (cat no. MV880/40; Chroma Technology Corp, Bellows Falls, VT) to create the Cy7 and IR870 illumination channels, respectively. These images are displayed in FIG. 28 and show the presence of TTF-1 expressing cells in the adenocarcinoma tissue (top right image) and p40 expressing cells in the squamous cell carcinoma image (bottom middle image), as expected. Also note the pink color, indicating mucin expression, in the cytoplasm of the TTF-1 expressing cells in the image of the mucicarmine stain recorded with the color camera (top left image), which is also associated with adenocarcinoma. The classification of adenocarcinoma of the lung is thereby confirmed by agreement between the special stain and IHC simultaneously on a single tissue section.

The invisible IHC may also be combined with non-staining techniques, such as phase contrast and differential interfering contrast (DIC) microscopy. As commonly performed, the visible spectrum would be used for the contrast-enhanced imaging and would be unaffected by the presence of the invisible chromogen absorbance.

It may be noted in the examples presented herein that spectral separation between the H&E or PAP stains and each of the invisible chromogens was large enough that images viewed or recorded with either visible white light or invisible LED illumination was sufficient to effectively separate each dye from the others, with little evidence of spectral crosstalk. However, residual amounts of crosstalk could be removed by application of spectral unmixing algorithms, for example, to remove some of the deep blue/UV absorbance of H&E stain to provide higher stain contrast for the deep blue/UV absorbing chromogens such as DCC, HCCA and AMCA.15,20,21 In addition, spectral crosstalk will allow more invisible dyes to be used in multiplex IHC since adding more dyes will increase spectral overlaps and require correction. With spectral unmixing, chromogens with absorbance closer to the H&E or PAP absorbance could be used and reference spectra of hematoxylin and eosin individually could be included with reference spectra for each chromogen in unmixing to remove invisible chromogen absorbance from the H&E stain. Further, with spectral unmixing, the hematoxylin and eosin components of H&E can be reduced to allow chromogens with absorbance closer to the hematoxylin or eosin to be used. The H&E image was then reconstructed as a two-dye composite image, free of chromogen absorbance, with individually adjustable intensifies of the hematoxylin and eosin components to match the pathologist's preference. Reduction of hematoxylin and eosin staining may alter the general appearance and interpretability of well know H&E staining, so this would be performed in a tradeoff with the ability to increase the multiplexing level of the IHC.

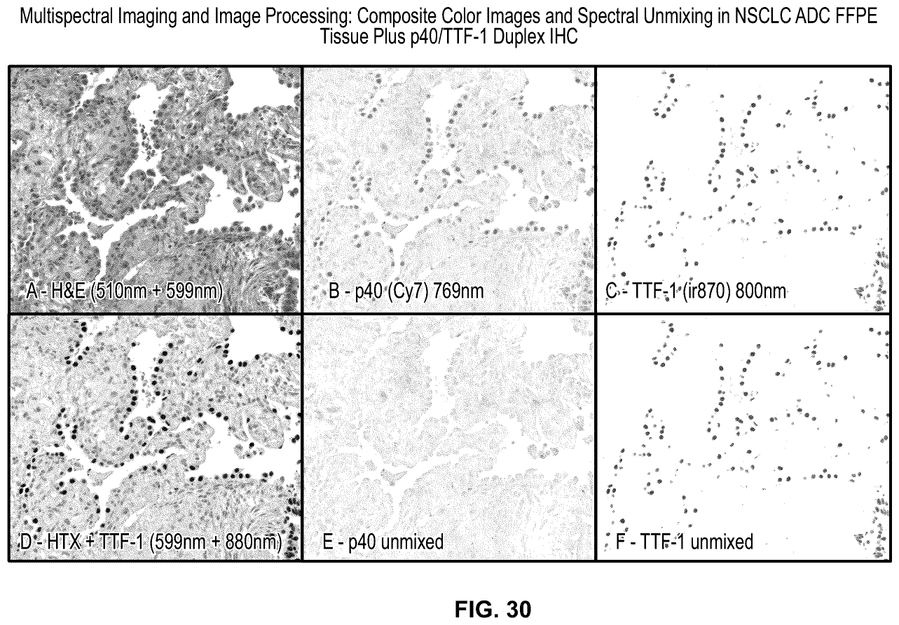

Example 13 and FIG. 28 describe duplex invisible IHC used with mucicarmine special stain, which requires that both invisible chromogens absorb light in the near-IR portion of the spectrum, due to strong mucicarmine special stain absorbance in the UV. This results in significant absorbance of the IR870 chromogen at the wavelengths that Cy7 absorbance is monitored (about 45% of IR870 absorbance at the 880 nm light channel is observed in the 769 nm light channel). The spectral crosstalk of IR870 into the Cy7 channel in FIG. 28 (top center panel) is not so apparent; however, crosstalk of more darkly stained TTF-1 positive cells is noticeable as shown in the top middle panel of FIG. 30. This slide is from the same NSCLC adenocarcinoma specimen shown in FIG. 28, stained by the same duplex IHC procedure but manually stained with H&E instead of mucicarmine special stain. Multispectral images were recorded with the monochrome camera illuminated with four filtered tungsten lamp channels: 510 nm (predominantly eosin), 599 nm (hematoxylin), 769 nm (predominantly Cy7), and 880 nm (IR870). The color H&E image (FIG. 30, Panel A) was prepared from the unmixed 510 nm and 599 nm images, reproducing the view through the oculars. The images recorded using the 769 nm light channel (predominantly C7 absorbance; FIG. 30, Panel B) and 880 nm light channel (IR870 absorbance; FIG. 30, Panel C) were processed to provide the unmixed p40 and TTF-1 images (FIG. 30, Panels E and F, respectively). As evidenced in FIG. 30 Panel B, the particularly dark staining IR870 cells selected for this example (possibly normal TTF-1 positive cells) have noticeable crosstalk into the 769 nm light channel which is eliminated after unmixing (FIG. 30, Panel E). Unmixed IR870 (FIG. 30, Panel F) shows little effect of unmixing due to minimal absorbance of other dyes at 880 nm. FIG. 30, Panel D shows a color composite produced from combining only the unmixed hematoxylin and TTF-1 images, mimicking a conventional single IHC using hematoxylin counterstain. As expected, p40 staining is absent in the adenocarcinoma tissue.

### Example 14. Image Processing to Reduce Interference Between Neighboring Stain Absorbance—Spectral Unmixing

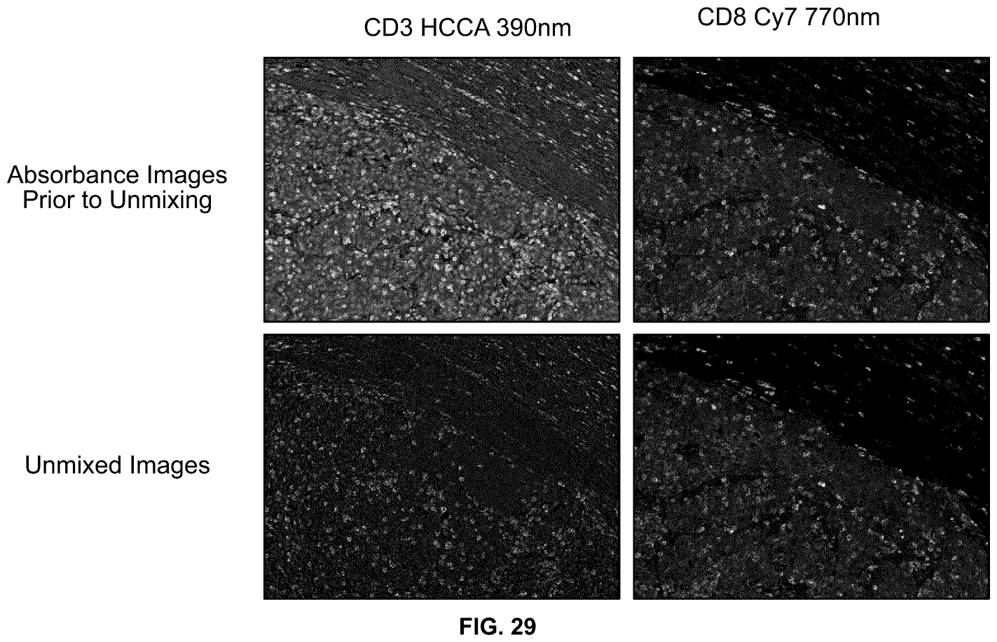

Image processing can be applied to recorded images of chromogens and conventional stains to reduce spectral crosstalk and provide images of individual chromogens and stains with reduced or eliminated interference from other chromogens and stains used in multiplexed assays. For example, in the top left transmitted light image of FIG. 16 the HCCA chromogen staining of regions of CD3 expression are clearly distinguished. However, the staining of all cellular nuclei is also faintly visible due to the broad absorbance of the hematoxylin conventional stain, which extends into the 390 nm light channel used to image HCCA. This is more clearly seen when transmission is converted to absorbance, as shown in the top left image of FIG. 29, as is typically done when performing quantitative analysis. Unmixing uses the relative absorbance of each individual dye in each light channel to correct for overlapping dye absorbance. This is process is described in detail in Morrison L E, Lefever M R, Behman L J, Leibold T, Roberts E A, Horchner U B, Bauer D R (2020) Brightfield Multiplex Immunohistochemistry with Multispectral Imaging. Lab Invest. https://doi.org/10.1038/s41374-020-0429-0 and references therein. The relative absorbance of each dye, determined from IHC of each dye individually, form crosstalk coefficients of a matrix, which upon inversion, provide the correction coefficients. The correction coefficients are multiplied by the various absorbance images of a multi-color stained microscope field, which are then added together to create the images of the pure stains with crosstalk removed. The results for the HCCA and Cy7 images are shown in FIG. 29, with the top two rows being the images prior to crosstalk correction (see corresponding transmitted light images in FIG. 16) and the bottom two rows being images after crosstalk correction. The unmixed HCCA shows considerable background reduction by suppression of the hematoxylin-stained nuclei. The Cy7 images do show much different because the hematoxylin has little or no absorbance in the far-red/near-IR portion of the spectrum where Cy7 absorbs.

Although spectral crosstalk may be small enough not to interfere with visual interpretation of transmitted light through the microscope, for example as depicted in FIG. 16, quantitative analysis of the absorbance images may benefit, for example, by making application of thresholds to distinguish regions of different protein expression more effective. This increases the accuracy of enumerating different cellular population, measuring intracellular distances, measuring protein expression levels, etc. Applying spectral unmixing in multiplex assays permits higher level multiplexing in digitally evaluated assays since the increased spectral crosstalk, that accompanies the use of more dyes, can be corrected.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the disclosure herein has been described with reference to particular embodiments, it is to be understood that these embodiments are merely illustrative of the principles and applications of the present disclosure. It is therefore understood that numerous modifications may be made to the illustrative embodiments and that other arrangements may be devised without departing from the spirit and scope of the present disclosure as defined by the appended claims. Sudan black B, Sudan III, Sudan IV, Tetrachrome stain (MacNeal), Thionine, Toluidine blue, Weigert 1878, Wright stain, and combinations thereof.

9. The method of claim 1, wherein the labeling of the first biomarker with the first detectable moiety comprises: (a) contacting the biological specimen with an anti-biomarker primary antibody; (b) contacting the biological specimen with an anti-specifies secondary antibody specific to the anti-biomarker primary antibody, wherein the anti-species antibody is conjugated directly or indirectly to at least one enzyme; and (c) contacting the biological specimen with a first detectable conjugate comprising (i) the first detectable moiety, and (ii) a tyramide moiety, a quinone methide moiety, or a derivative or analog of a tyramide moiety or quinone methide moiety.

10. The method of claim 1, wherein the labeling of the first biomarker with the first detectable moiety comprises: (a) contacting the biological specimen with an anti-biomarker primary antibody; (b) contacting the biological specimen with an anti-specifies secondary antibody specific to the anti-biomarker antibody, wherein the anti-species antibody is conjugated directly or indirectly to at least one enzyme; (c) contacting the biological specimen with a first tissue reactive conjugate comprising: (i) a first member of a pair of reactive functional groups capable of participating in a click chemistry reaction, and (ii) a tyramide moiety, a quinone methide moiety, or a derivative or analog of a tyramide moiety or quinone methide moiety; and (d) contacting the biological specimen with a detectable conjugate comprising (i) the first detectable moiety, and (ii) a second member of the pair of reactive functional groups.

11. The method of claim 1, wherein the first biomarker is a protein biomarker.

12. (canceled)

13. The method of claim 1, wherein the first biomarker is a nucleic acid biomarker.

14. The method of claim 1, wherein the first detectable moiety comprises one of a coumarin core, a heptamethine cyanine core, and a croconate core.

15. The method of claim 8, further comprising labeling a second biomarker with a second detectable moiety, wherein the second detectable moiety has a FWHM of less than about 70 nm and an absorbance maximum (λₘₐₓ) of either less than about 430 nm or greater than about 670 nm, and wherein the first and second detectable moieties are different.

16. (canceled)

17. (canceled)

18. (canceled)

19. (canceled)

20. (canceled)

21. A method of detecting one or more targets within a biological specimen disposed on a substrate, comprising:
(a) labeling a first biomarker marker with a first detectable moiety, wherein the first detectable moiety comprises one of a coumarin core, a heptamethine cyanine core, and a croconate core; and
(b) staining the biological specimen disposed on the substrate with at least one conventional dye having one or more peak absorbance wavelengths between about 400 nm and about 700 nm, wherein the peak absorbance wavelength of the first detectable moiety and the one or more peak absorbance wavelengths of the one or more conventional dyes are separated by at least 20 nm.

22. (canceled)

23. (canceled)

24. The method of claim 21, wherein the first detectable moiety has a peak absorbance wavelength of less than about 430 nm.

25. (canceled)

26. The method of claim 21, wherein the first detectable moiety has a peak absorbance wavelength of greater than about 670 nm.

27. (canceled)

28. (canceled)

29. (canceled)

30. The method of claim 21, further comprising labeling a second biomarker with a second detectable moiety, wherein the second detectable moiety has a FWHM of less than about 200 nm and an absorbance maximum (λₘₐₓ) of either less than about 430 nm or greater than about 670 nm, and wherein the first and second detectable moieties are different.

31. (canceled)

32. (canceled)

33. (canceled)

34. (canceled)

35. (canceled)

36. The method of claim 21, wherein the first detectable moiety is selected from the group consisting of: 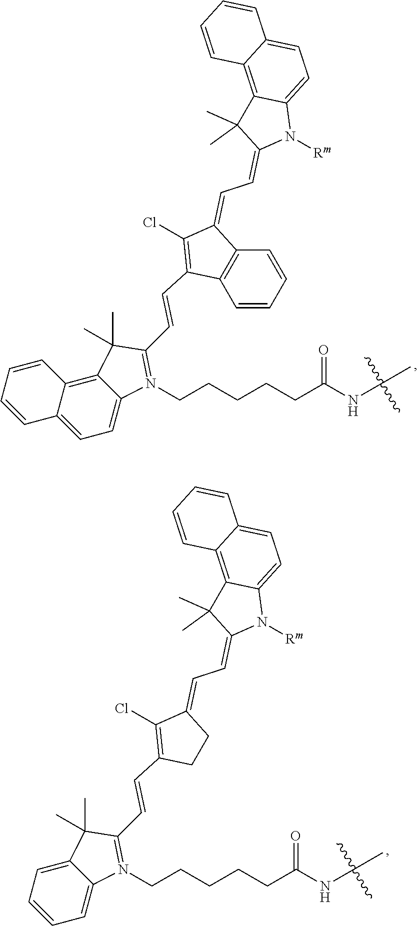 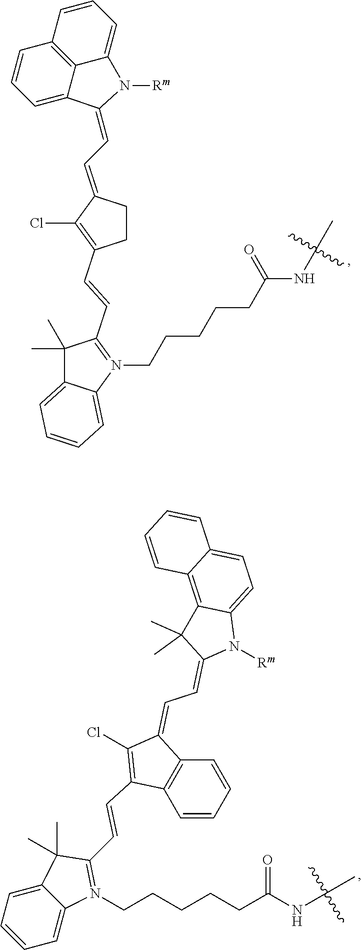 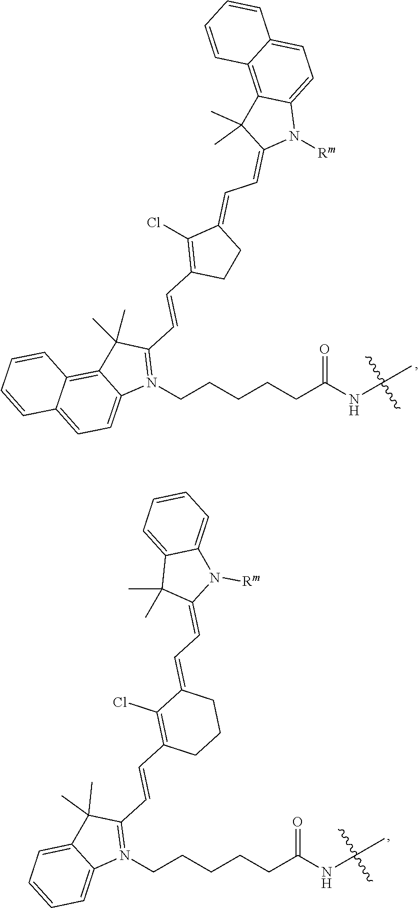 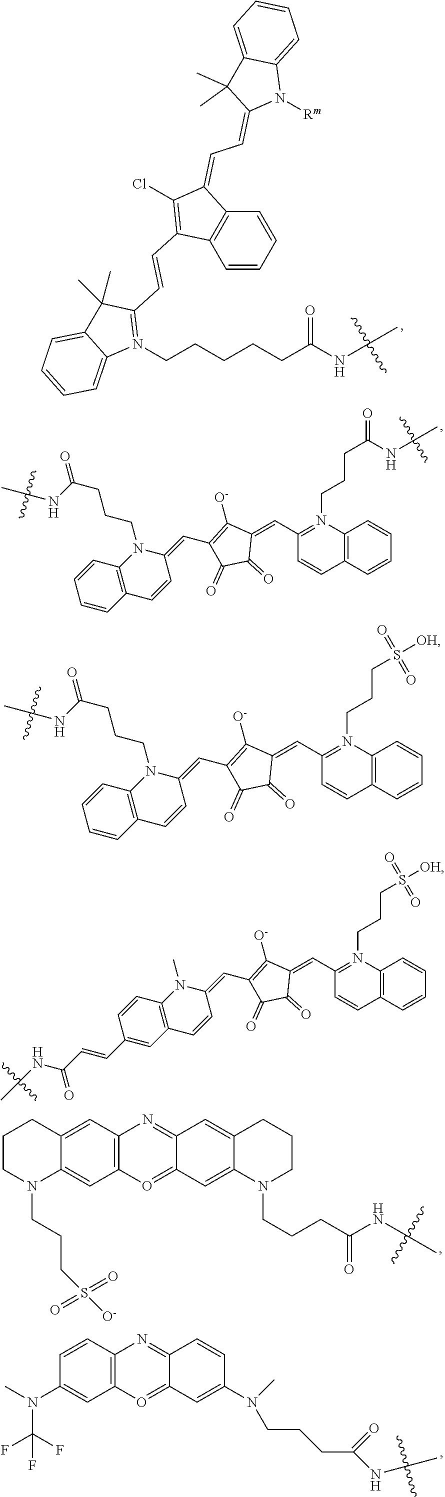 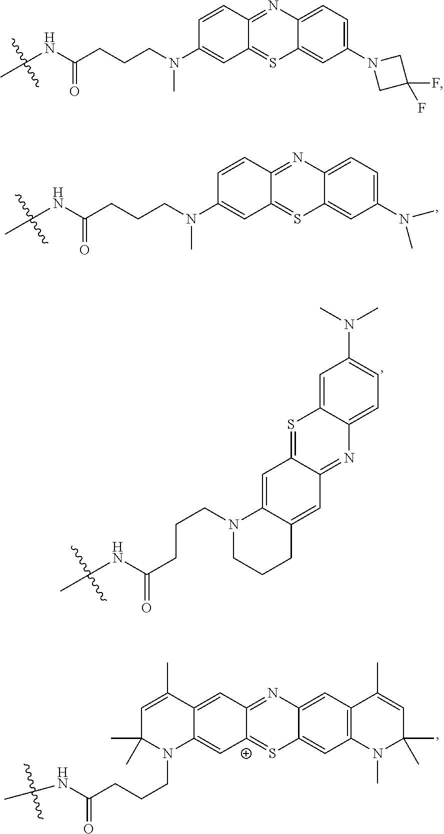 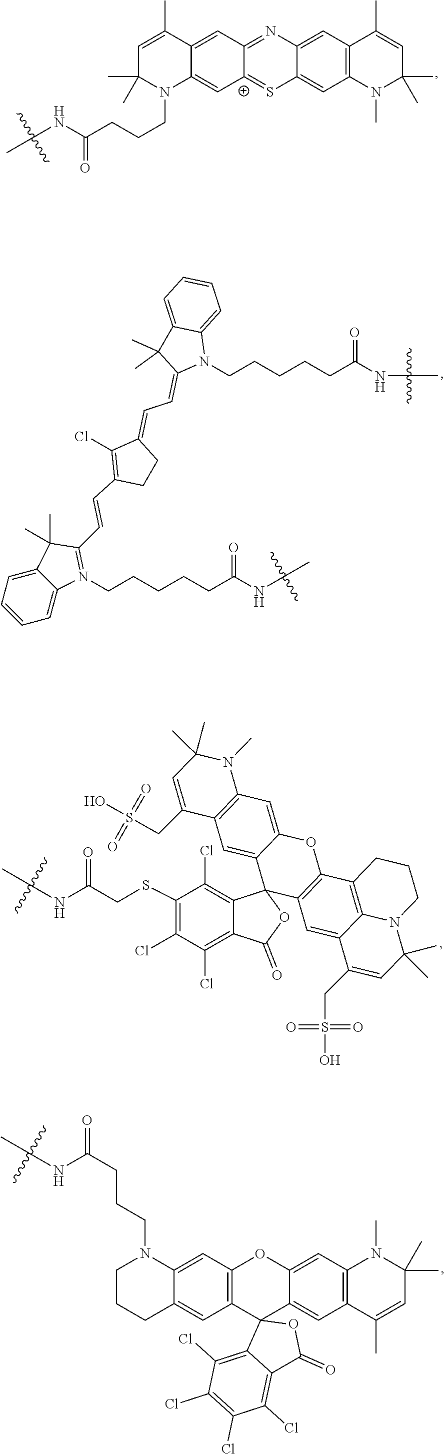 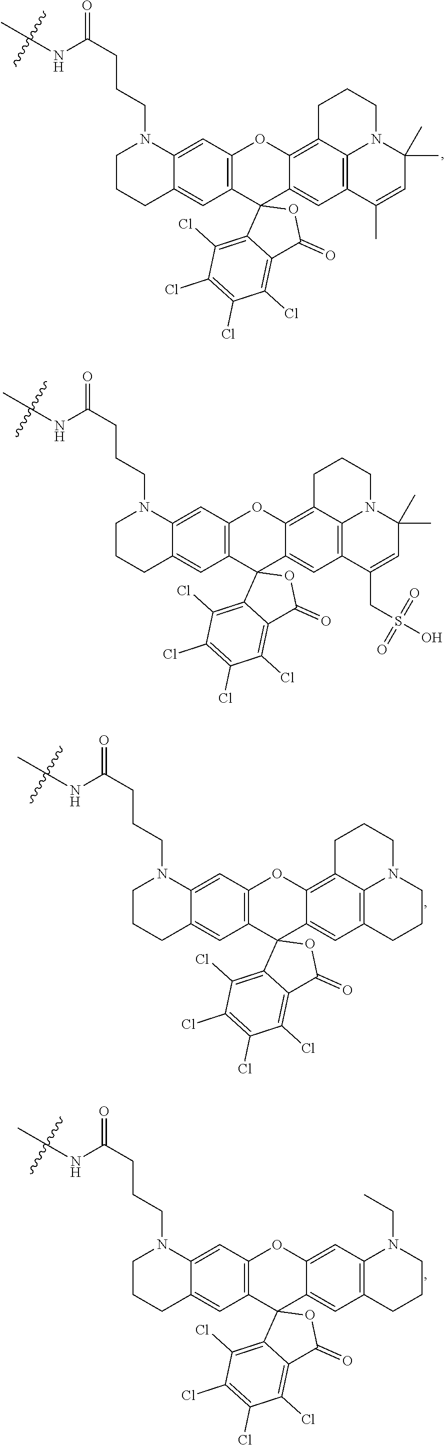 [SMILES: *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21 ; *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC] 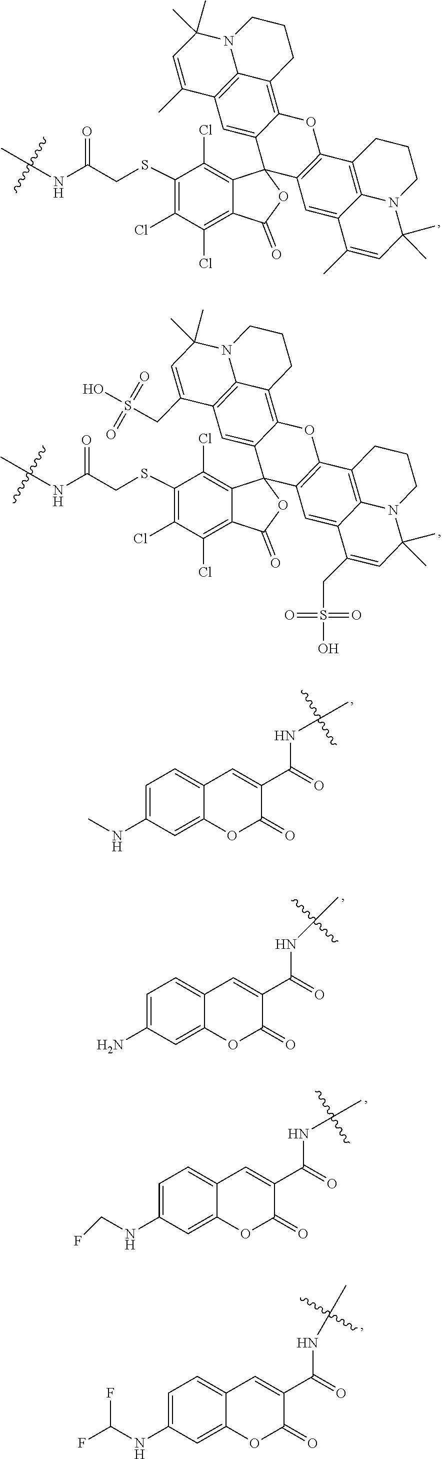 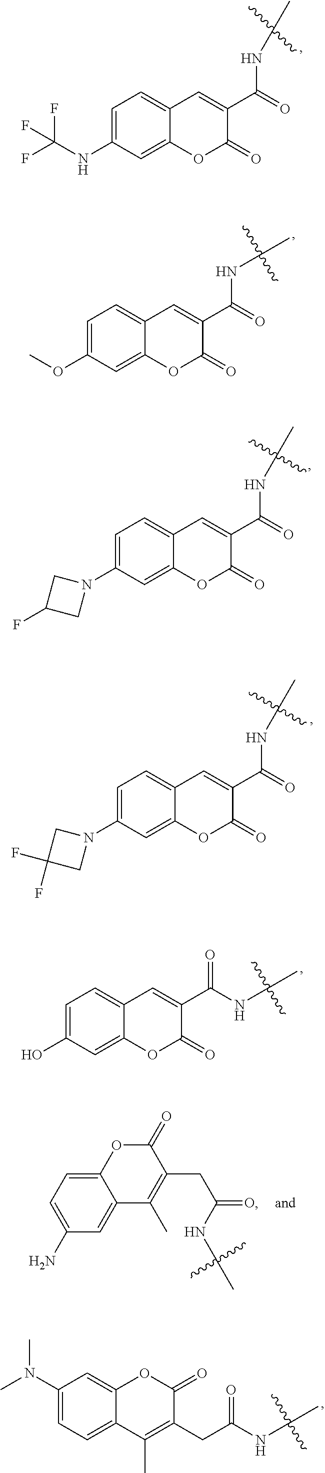
where the symbol “ [symbol] ” refers to the site in which the detectable moiety is conjugated to another moiety of a detectable conjugate.

37. A stained biological specimen disposed on a substrate comprising a first biomarker labeled with a first detectable moiety; wherein the first detectable moiety has a FWHM of less than about 200 nm and an absorbance maximum (λₘₐₓ) of less than about 430 nm or greater than about 670 nm; wherein the stained biological specimen is stained with at least one conventional dye, wherein the at least one conventional dye has one or more peak absorbance wavelengths within the visible spectrum.

38. (canceled)

39. (canceled)

40. (canceled)

41. (canceled)

42. (canceled)

43. The stained biological specimen of claim 37, wherein the at least one conventional dye comprises hematoxylin and eosin.

44. The stained biological specimen of claim 37, wherein the at least one conventional dye is selected from the group consisting of Acid fuchsin, Alcian blue 8 GX, Alizarin red S, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, Cresyl violet, Crystal violet, Darrow red, Ethyl green, Fast green F C F, Fluorescein Isothiocyanate, Giemsa Stain, Indigo carmine, Janus green B, Jenner stain 1899, Light green SF, Malachite green, Martius yellow, Methyl orange, Methyl violet 2B, Methylene blue, Methylene blue, Methylene violet (Bernthsen), Neutral red, Nigrosin, Nile blue A, Nuclear fast red, Oil Red O, Orange G, Orange II, Orcein, Pararosaniline, Phloxin B, Protargol S, Pyronine B, Pyronine Y, Resazurin, Rose Bengal, Safranine O, Sudan black B, Sudan III, Sudan IV, Tetrachrome stain (MacNeal), Thionine, Toluidine blue, Weigert 1878, Wright stain, and combinations thereof.

45. The stained biological specimen of claim 44, wherein the stained biological specimen further comprises a second biomarker labeled with a second detectable moiety, wherein the second detectable moiety has a FWHM of less than about 200 nm and an absorbance maximum (λₘₐₓ) of either less than about 430 nm or greater than about 670 nm, and wherein the first and second detectable moieties are different.

46. The stained biological specimen of claim 45, wherein the second detectable moiety is within the infrared spectrum.

47. The stained biological specimen of claim 45, wherein the second detectable moiety is within the ultraviolet spectrum.

48-117. (canceled)
